(19) United States
(12) Patent Application Publication
(10) Pub. No.: US 2025/0360263 A1
(43) Pub. Date: Nov. 27, 2025

(54) DRUG DELIVERY DEVICE FOR DELIVERING AT LEAST ONE DRUG, OPTIONALLY AT LEAST ONE RECONSTITUTED DRUG, MEDICAL DEVICE AND MEDICAL SYSTEM

(71) Applicant: GENZYME CORPORATION, Cambridge, MA (US)

(72) Inventors: Alexander Hee-Hanson, Melbourne (GB); Tom Lever, Melbourne (GB); Craig Nelson, Melbourne (GB); Haiming Wu, Cambridge, MA (US)

(73) Assignee: GENZYME CORPORATION, Cambridge, MA (US)

(21) Appl. No.: 18/674,045

(22) Filed: May 24, 2024

(51) Int. Cl. A61M 5/142; A61M 5/172

(52) U.S. Cl. CPC A61M 5/142; A61M 5/172

(57) ABSTRACT

Disclosed is a drug delivery device (DD1) for delivering at least one drug (D), comprising:
A case (CAD),
At least one interface portion (IFP2) being part of an interface (IF) to a medical device (MD1), and
A retaining space (RS2) within the case (CAD), the retaining space (RS2) configured to receive a fluid guide system (FGS2) or comprising the fluid guide system (FGS2).

The disclosure relates to a drug delivery device for delivering at least one drug, optionally at least one reconstituted drug.

There is an ever increasing demand for handling of and delivering of drugs, e.g. of lyophilized drugs and/or of individualized drug mixtures.

The demand is driven e.g. by the fact that more and more drugs are lyophilized, e.g. biomedical drugs, or other drugs, e.g. insulin. In short, lyophilization is a freeze drying process in which a liquid drug goes through at least one cycle of freezing and/or sublimation in a vacuum to become solid, e.g. at sub-freezing temperatures, e.g. at temperatures below the freezing point of the liquid drug. After the drugs have first been frozen, a vacuum reduces the pressure until sublimation takes place, e.g. the transition of a substance directly from the solid state to the gas state, without passing through the liquid state. The advantage is that the lyophilized drugs may be transported more easily and may be stored for longer times compared to e.g. liquid drug formulations. Thus, the lyophilization industry is a steady growing industry. Correspondingly, the demand for reconstitution devices increases steadily too.

Associated with lyophilized drug, there may be the challenge to prepare them prior to use when reconstitution is required. This would be even more so when multiple vials or multiple other drug containers are needed, or when a variable amount of drug is needed, e.g. such as weight based dosing. Moreover, the rate of drug delivery may be relevant as well. The current invention may address at least one of these issues.

Similar problems may arise if different types of drugs have to be mixed for special patients.

Delivery of the reconstituted and/or mixed drug(s) should be easy for the HCP (Health Care Professional) and/or for the patient. Especially, the delivery of the drug(s) should be as comfortable as possible.

### SUMMARY

A drug delivery device for delivering at least one drug is provided, comprising:
A case, and/or
At least one interface portion being part of an interface to a medical device, and/or
A retaining space within the case, the retaining space may be configured to receive a fluid guide system or may comprise the fluid guide system, and/or
   wherein the case may be configured to receive or may comprise at least one drug container,
   wherein the at least one interface portion may comprise at least one fluidic port of the fluid guide system, and/or
   wherein the fluid guide system may be fluidically coupled to or may be configured to be fluidically coupled to the at least one drug container, and/or
   wherein the drug delivery device may be detachably connectable to the medical device via the at least one interface portion, e.g. in order to transfer the at least one drug from the medical device to the at least one drug container.

A medical device for drug handling is provided, comprising:
A (further) fluid guide system for guiding the at least one drug during drug handling, and/or
An electronic control unit, and/or
An interface portion for detachable coupling of the drug delivery device according to the embodiment mentioned above to the medical device, and/or
   wherein the electronic control unit may be configured to control the fluid flow within the (further) fluid guide system of the medical device during the handling of the at least one drug, and/or
   wherein optionally the handling of the at least one drug may comprise at least one reconstitution procedure of the at least one drug, and/or
   wherein optionally the at least one interface portion may comprise at least one outflow fluid port which is configured to transfer the at least one drug to the drug delivery device after handling of the at least one drug.

A system for handling at least one drug and for delivery of the at least one drug is provided, comprising:
A medical device according to the embodiment mentioned above for handling the least one drug, and/or
A drug delivery device according to the embodiment mentioned above for delivering the at least one drug.

A system, e.g. a kit for handling at least one drug and for delivery of the at least one drug may comprise:
a medical device for handling the least one drug, and
a drug delivery device for delivering the at least one drug,
   wherein the medical device may comprise:
at least one interface portion of the medical device for detachable coupling of the drug delivery device,
   wherein the handling of the at least one drug may comprise at least one reconstitution procedure of the at least one drug, and
   wherein the at least one interface portion of the medical device may comprise at least one outflow fluid port which is configured to transfer the at least one drug to the drug delivery device after handling of the at least one drug, and
   wherein the drug delivery device may comprise:
a case,
at least one interface portion of the drug delivery device being part of an interface to the medical device,
   wherein the at least one interface portion of the drug delivery device may comprise at least one inflow port of a fluid guide system of the drug delivery device, and
   wherein the drug delivery device may be detachably connectable to the medical device via the at least one interface portion of the drug delivery device in order to transfer the at least one drug from the medical device to the at least one drug container via the at least one inflow port.

It is an object of the disclosed embodiments to provide a drug delivery device. The drug delivery device should especially be simple and/or small and/or safe and/or should allow sterile delivery of the drug, especially without (manual) cleaning of the drug delivery device and/or of parts of the drug delivery device. Furthermore, a corresponding medical device and a corresponding system should be provided as well as further items mentioned below.

This object is solved by the drug delivery device according to claim 1. Further embodiments are mentioned in the dependent claims.

According to an embodiment, the drug delivery device for delivering at least one drug may comprise:
A case, and/or
At least one interface portion being part of an interface to a medical device, and/or
A retaining space within the case.

According to an embodiment, the retaining space may be configured to receive a fluid guide system or may comprise the fluid guide system. The fluid guide system may comprise at least one fluidic channel, e.g. an inflow channel and/or an outflow channel.

According to an embodiment, the case may be configured to receive or may comprise at least one drug container. The drug container may be a flexible bag, a rigid cartridge or a syringe. The flexible bag may be stretchable, e.g. an elastomeric bag. Alternatively or according to an embodiment, the flexible bag may be non-stretchable.

According to an embodiment, the at least one interface portion may comprise at least one fluidic port of the fluid guide system. The fluidic port may be an inflow port, e.g. comprising a pierceable septum and/or comprising a self-sealing connector. The fluidic port may comprise a sterilization means, e.g. a sponge which may comprise or may be exposed to an alcoholic solution or to another sterilizing solution.

According to an embodiment, the fluid guide system may be fluidically coupled to or may be configured to be fluidically coupled to the at least one drug container. The fluid guide system may be integrally connected to the at least one drug container, e.g. in order to enable a tight and/or sterile fluidic connection. Alternatively or according to an embodiment, the drug container and the fluid guide system may be at least two different parts that are produced separately. There may be an assembly step that connects both components, e.g. using an appropriate type of fluidic connection.

According to an embodiment, the drug delivery device may be detachably connectable to the medical device via the at least one interface portion, e.g. in order to transfer the at least one drug from the medical device to the at least one drug container and/or to remove the drug delivery device from the medical device in same application scenarios thereafter. Thus, the drug delivery device may be docked to the medical device and/or undocked from the medical device, e.g. several times, optionally at least once, twice or more than twice dock/undock steps may be possible. The number of dock/undock steps may be in the range of 1 to 1000 or 10 to 500 or 20 to 200.

According to an embodiment, the fluid guide system and/or the at least one drug container may be replaceable. Thus, sterility may be provided without cleaning of the drug delivery device, e.g. of the case of the drug delivery device.

According to an embodiment, the at least one drug container may comprise at least one flexible portion, e.g. an enclosure portion or a sidewall portion, which may define at least a portion of at least one reservoir within the drug container. The reservoir may form a chamber that is configured to receive the at least one drug, e.g. after drug handling in a medical device.

The at least one drug container may comprise a flexible material or may essentially be formed of a flexible material, e.g. by more of 90 percent per volume or per mass. Thus, the at least one drug container may be a flexible container, e.g. a flexible bag. The flexible material may be a material comprising of consisting of a plastic material, e.g. comprising or consisting of poly vinyl chloride (PVC), or other polymers, Ethylene-vinyl-acetate (EVA), or other copolymers, Polypropylene (PP), or other polyolefins, etc. One layer of plastic material may be used. Thus, cyclo-olefin polymers and/or cyclo-olefin copolymers (COP/COC)” may be used, e.g. for more sensitive drug products.

According to an embodiment at least two layers may be used to form a flexible side wall of the drug container.

Flexible materials may have the technical effect that storage room for storage of the drug container(s) may be reduced considerably. Thus, several plastic bags may be rolled to form a roll of plastic bags. Moreover, a flexible material may comprise only a small amount of plastic material. Thus, the waste impact may be low. Moreover, the patient/user may use bags easily.

Thermoplastic materials may be used, e.g. allowing plastic welding, e.g. using higher temperature and/or ultrasonic. Thus, e.g. a plastic bag may be simply formed by welding two plastic sheets together or by sealing at least one side face of a folded plastic sheet.

The plastic bag or other flexible container may have at least one fluid port, e.g. at least one of the following:
An inflow port,
An outflow port,
A combined fluid input/output port, etc.

The plastic bag or other flexible container may carry at least one label, e.g. comprising a bar code, a QR (Quick Response) code or other machine readable code and/or comprising text readable by humans.

The same technical features may apply to at least one drug container within the medical device, e.g. comprising also at least one flexible chamber portion forming at least a portion of a sidewall or the complete sidewall of the drug container.

The drug container may be replaceable relative to the case. Thus, a clean container may be used any time it is needed or recommendable. The maximum time of usage of a drug container may be one day or 12 hours or less. A single use of the drug container may be a preferred option.

The drug delivery device may comprise at least one pump, e.g. exactly one pump. However, delivery of the drug using gravity may also be possible. A combination of a pump and of delivery by gravity may be used as well. The at least one pump may be used for delivering of the drug. A pumping device may perform several repeated pump movements, e.g. rotary movements or translatory movements into opposite directions. At least one force generating unit for generating a force for delivering the drug out of the at least one drug container into the body of a patient may be used as well. The force generating unit may not perform cyclic pumping movements. The force generating unit may perform a translation of a plunger into only one direction or the deflation of balloon comprising the at least one drug.

The drug may be a reconstituted drug and/or a drug that has been mixed using several different drugs, e.g. at least two different drugs, at least three different drugs, etc. The number of different drugs may be in the range of 1 to 100 or in the range of 3 to 50 or in the range of 5 to 30.

The drug delivery device may comprise an electronic control unit, e.g. a second electronic unit in addition to a first electronic unit within the medical device. However, purely mechanically operating drug delivery devices may be used as well. If the drug delivery device comprises an electronic control unit, there may be a communication unit which may allow electronic data transfer between the drug delivery device and the medical device, e.g. in order to provide advanced features for the user and/or for drug delivery. The electronic control unit ECU of the drug delivery device may comprise at least one processor, e.g. a digital processor and/or at least one digital memory. However, alternatively, a finite state machine may be used as well.

The fluid guide system may be replaceable as well. Thus, it may be sterile and/or, no cleaning may be necessary. The fluid guide system may comprise or may be e.g. a cartridge or a flexible bag. According to an embodiment, the fluid guide system may comprise an inflow channel and/or port and an outflow channel and/or port.

According to a further embodiment, the fluid guide system may be replaceable and the at least one drug container may be replaceable. Thus, no cleaning of these parts may be necessary and sterilized parts may be used whenever necessary.

The drug container may have a maximum filling volume, e.g. within the range of 10 ml (milliliter) to 1000 ml or in the range of 20 ml to 600 ml or in the range of 30 ml to 300 ml or in the range of 50 ml to 150 ml.

According to an embodiment, the drug delivery device (DD1) may comprise not only the fluidic interface portion but also a data transmission interface portion. The data transmission interface portion may comprise a data receiving and sending unit which may be configured to communicate with the medical device. The drug delivery device may be configured to receive at least one of the following data from the medical device:
Amount of drug/dose to be delivered, and/or
Rate of drug delivery, and/or
Data indicating that the drug delivery device is filled, and/or
Data identifying a patient, e.g. comprising a patient identifier, and/or
Data requesting a value of the battery level in the drug delivery device.

Exemplary, the health care personal, e.g. a nurse may compare the patient identifier with an identifier on the patients wrist, e.g. stored within the vital sign monitor device as mentioned above. The comparison may be done manually or automatically, e.g. using a bar code or QR (quick response) code which is based on the patient identifier. The code may be read by a bar code reader or QR code reader (e.g. comprising a camera) within the medical device and/or within the vital sign monitoring device (e.g. smart watch) and/or within the drug delivery device.

The patient identifier may also be stored in an electronic health record (EHR). The electronic health record may be stored in a cloud based platform or within a cloud based platform device. The patient identifier may be used to make sure that the correct patient will receive the correct type of drug(s) and/or the correct amount of drug(s) and/or at the correct time and/or with the correct drug delivery rate, etc.

Thus, enhanced application scenarios may be possible as well as enhanced usability.

According to an embodiment, the drug delivery device may be configured to send at least one of the following data to the medical device:
Confirmation data that confirms that the delivery of the at least one drug has been successful, and/or
Start time and end time of the delivery of the at least one drug, and/or
Start time and duration of the delivery of the at least one drug, and/or
Data indicating the actual amount of drug delivered, and/or
Data indicating the rate of drug delivery, and/or
Data indicating the type of drug(s) delivered, and/or
Data identifying a patient, e.g. comprising a/the patient identifier, and/or
Data specifying the battery level, e.g. the remaining battery level in the drug delivery device, and/or
Error data indicating an error during the delivery of the at least one drug, and/or
Data demanding filling of the at least one drug container.

Thus, again, enhanced application scenarios may be possible as well as enhanced usability.

According to a further embodiment, the data transmission interface portion may be part of a wireless connection, e.g. of a radio frequency (RF) connection. Thus, the drug delivery device and a medical device may communicate via a greater range, e.g. via a distance of at least 3 m (meters), or of at least 5 meters. The distance may be less than 100 m, to give only one example for an upper limit.

According to a further embodiment, the data transmission interface portion may be part of a physical, e.g. wired connection. Thus, a USB connection may be used, e.g. for transmitting data and/or for transmitting power. The physical data transmission interface portion may be one of the at least one interface portion of the drug delivery device. The physical connection may be implemented in a simpler way than e.g. a wireless connection.

According to a further embodiment, the at least one interface portion may comprise an electric power port which is configured to receive electrical energy from the medical device. Thus, there may be at least two interface portions or at least three different interface portions. The usability of the drug delivery device may be raised by each interface portion.

According to a further embodiment, a combined electric power port and physical data transmission port may be used, e.g. an USB port (Universal Serial Bus). Thus, a USB C-port may be used according to USB 3.0 and/or 3.1 or any other appropriate USB port. USB standards of a lower number, e.g. USB 2.0 of a higher number, e.g. defined in the future may be used as well.

According to an embodiment, the fluid guide system and/or the at least one drug container may be replaceable. Technical effects thereof have been mentioned already above, e.g. sterility.

According to an embodiment, the case may comprise a convex coupling portion which is configured to complement a shape of a concave support portion of the medical device. The convex coupling portion may extend along a length that is at least half or at least three quarter the lengths of the drug delivery device. At least one or at least two of the at least one interface portion may be arranged on the convex coupling portion. The convex coupling portion may allow secure holding of the drug delivery device within a support portion of the medical device. Moreover, the convex coupling portion may promote coupling of the other interface portions.

According to an embodiment, the drug delivery device may be coupled to or may be configured to be coupled to a catheter or to an interface to a catheter. The drug delivery device may be configured to infuse the at least one drug into a vessel of the body of a patient. Thus, intravascular infusion may be used, e.g. into blood vessels. Intravenous infusion may be preferred from a medical point of view. However, there may also be reasons for intra-arterial infusion. Infusion into an artery may involve a comparably high risk of injury and massive loss of blood. Infusion into a vein may involve a much lower risk of injury and/or lower loss of blood due to the lower blood pressure within veins compared to the blood pressure within arteries.

The infusion time may be in the range of 1 min (minute) to several hours (e.g. less than 10 hours) or in the range of 3 min to 1 hour or in the range of 1 min to 20 minutes or in the range of 5 minutes to 20 minutes. The infusion time may be more than, e.g. 1 min, 5 min, 10 min and/or less than 5 hours, 4 hours, 3 hours, 2 hours 1 hour to give only some examples.

The drug may be a reconstituted drug. Reconstitution is defined in the definition part of this document. Thus, simple and/or automated reconstitution may be performed by the medical device. Automatic transfer of the reconstituted drugs may take place from the medical device to the drug delivery device. User involvement for reconstitution and/or drug delivery may be low. The same may apply to mixing of several drugs without reconstitution or with reconstitution of at least one of the drugs which have to be mixed.

According to an embodiment, the drug delivery device may be configured to receive mechanical energy from the medical device. The drug delivery device may comprise a storage element or may be configured to comprise a storage element. The storage element may be configured to store the received mechanical energy. Thus, a simple mechanism may be used to power a mechanical pump or a mechanical force generating unit. Electrical power may not be used in the drug delivery device or electrical power may only be used for data communication. Thus, the drug delivery device may be a simple device. If no electrical power is used in the drug delivery device, requirements for fulfilling standards with regard to electronic parts may be lower, e.g. with regard to recycling of batteries. Another relevant standards that may not apply to mechanical devices is e.g. the RoHS directive (Restriction of Hazardous Substances) of the EU (European Unit).

According to an embodiment, the drug delivery device may be configured to receive the mechanical energy during the transfer of the at least one drug from the medical device to the drug delivery device via the generated fluid pressure of a fluid comprising the at least one drug. The drug delivery device may be configured such that the fluid pressure loads the mechanical storage element. Thus, the drug delivery device may be simplified even more.

Optionally, the mechanical storage element may comprise at least one mechanical spring that is configured to store the transferred energy. A spring is one of the simplest mechanical devices. Moreover, springs may be easily adapted to the intended use case, e.g. by selection of an appropriate material and/or shape.

Optionally, the mechanical storage element may be an inflatable drug container that is configured to be inflated by the pressure. The resiliency of e.g. an inflatable bag may be tailored to the intended use scenario. The drug delivery device may comprise a balloon pump. The case may e.g. protect the balloon/bag and/or may ease filling of the inflatable drug container.

Optionally, the spring may be configured to be loaded, e.g. compressed or tensioned by the pressure used to transfer the at least one drug. The spring may be a metal spring, e.g. helical spring. A compression spring or a tension spring may be used. The spring may not come into contact with the drug(s), e.g. it must not be cleaned after each use. However, a plunger or other member which is driven by the spring may also be not in contact with the spring, e.g. if a plug or other fluidic isolating member is used between the plunger and the fluid/drug.

No electrical energy storage element may be necessary in the drug delivery device or at least not an electrical energy storage element that drives an electrical pump. Thus, an electrical energy storage element may be used only for control purposes and/or for communication purposes or other purposes that are different from direct drug delivery. Thus, the medical device may be made small and/or simple.

According to a further embodiment, pumping and/or other delivery of the fluid in the drug delivery device may be purely mechanically.

According to a further embodiment, e.g. a balloon may be inflated by the fluid which is transferred from the medical device to the drug delivery device. Optionally a membrane or an elastomeric consumable may be used for storing of the drug. Thus, the principle of an elastomeric pump may be used, e.g. a balloon which is inflated by the drug solution. The balloon may deflate, i.e. decrease its inner volume if the drug is dispensed. Thus, the balloon may pump or expel the drug to the outside, e.g. without usage of further pumping means.

Again, no electrical energy storage element may be necessary in the drug delivery device or at least not an energy storage element that drives an electrical pump, e.g. used only for control and/or data communication purposes. Thus, the medical device may be small. The pumping of fluid in the drug delivery device may be purely mechanically.

In both cases, e.g. spring and plunger or elastomeric balloon, no further interface portion may be necessary for the transfer of the mechanical energy because the fluidic interface portion may be used for the transfer of the mechanical energy as well. Thus, the drug delivery device may be a simple device.

According to an embodiment, the drug delivery device may be configured to receive the mechanical energy via a movable or rotatable solid element that may be configured to be driven by a driving element in the medical device. An example is described below in the description of the figures. The solid element may be e.g. mechanically coupled to the storage element in order to transfer movement or rotation from the solid element to the storage element. The storage element may comprise at least one spring that may be configured to be biased by a movement of the solid element. The storage element may be configured as a pump, e.g. as a peristaltic pump or may be configured to drive a pump.

A further interface portion may be used for the transfer of the mechanical energy. However, the further interface portion may be simple, e.g. providing an interface to a gear that is driven by an electrical motor. Thus, transfer of mechanical energy may be possible without using pressure of the drug solution. Thus, the transfer of mechanical energy may be independent of the transfer of drug(s).

Again, for the embodiment with a further mechanical interface portion, no electrical energy storage element may be necessary in the drug delivery device or at least not an energy storage element that drives an electrical pump. Thus, the medical device may be small and simple. Purely mechanical pumping and/or purely mechanical drug delivery may be used in the drug delivery device.

A mechanical energy transfer that is independent of the transfer of drug solution may have several advantages. Thus, this may open design freedoms and/or may enable different technical solution(s) and/or application scenarios.

According to a second aspect, a medical device for drug handling is provided. The medical device may comprise:
A fluid guide system for guiding the at least one drug during drug handling, and/or
An electronic control unit, and/or
An interface portion for detachable coupling of the drug delivery device according to any one of embodiments mentioned above to the medical device.

According to an embodiment, the electronic control unit may be configured to control and/or to guide the fluid flow within the fluid guide system of the medical device during the handling of the at least one drug. Valves and/or valve portions may be used to guide the fluid flow in different directions and/or to different channels within the fluid guide system.

According to a further embodiment, e.g. optionally, the handling of the at least one drug within the medical device may comprise at least one reconstitution procedure of the at least one drug. According to a further embodiment, mixing of at least two drugs may be performed in the medical device without reconstitution of drugs or with reconstitution of at least one drug which has to be mixed with other drugs or with at least one other drug.

According to a further embodiment, e.g. optionally, the at least one interface portion may comprise at least one outflow fluid port which may be configured to transfer the at least one drug, e.g. comprising at least one reconstituted drug, to the drug delivery device after handling of the at least one drug.

According to a further embodiment, the electronic control unit may be configured to control and/or to guide the fluid during a transfer of the at least one drug to the drug delivery device. This may simplify the drug delivery device. However, according to another embodiment, the drug delivery device may at least initiate and/or control the fluid flow from the medical device to the drug delivery device.

According to a further embodiment, the fluid guide system may comprise at least one drug port, at least two drug ports, at least three drug ports, etc. configured to be connected to a respective one of at least two drug containers comprising the at least one drug. The number of drug ports may be in the range of 1 to 100 or in the range of 2 to 50 or in the range of 3 to 30. The fluid guide system may comprise at least one outflow fluid port configured to dispense a fluid from the fluid guide system.

According to a further embodiment, the fluid guide system may be a removable and/or consumable fluid guide system. Thus, the fluid guide system may be replaced after each drug preparation, e.g. reconstitution procedure and/or mixing procedure.

The electronic control unit ECU of the medical device may comprise at least one processor, e.g. a digital processor and/or at least one digital memory. However, alternatively, a finite state machine may be used as well.

Although the drug delivery device may be detachable from the medical device, it is not excluded that the drug can be delivered when the drug delivery device is still connected to the medical device. This may allow long infusion, e.g. during the night and/or during the patient sleeps.

Thus, a system comprising the medical device and the drug delivery device, etc. may be configured to deliver the at least one drug when drug delivery device is still connected to medical device in a first mode of operation. The connection may be fluidically and/or mechanically. Several interface portions may be connected, e.g. a fluidic interface portion and/or an electrical power interface portion and/or a physical data transmission interface portion and/or a mechanical energy transfer interface portion as mentioned above.

In a second mode of operation of the system, the drug delivery device may be removed (detached) from the medical device for drug delivery, e.g. for short trips away and/or for a main infusion. There may be no physical connection between the drug delivery device and the medical device in the second operation mode. Only an optional wireless data transmission interface portion of the drug delivery device may still be “connected” to the medical device. Several other interface portions may be disconnected, e.g. a fluidic interface portion and/or an electrical power interface portion and/or a physical data transmission interface portion and/or a mechanical energy transfer interface portion as mentioned above.

The fluid guide system (FGS) of the medical device may be replaceable. Thus, the fluid guide system of the medical device may be sterile, e.g. if taken out of a sealed plastic bag. Moreover, no cleaning of the fluid guide system may be necessary, e.g. a cartridge or a flexible bag may be simply disposed after use. Thus, infection of the patient may be prevented. However, multi-use of a fluid guide system may be used as well, e.g. using automatic cleaning steps, optionally for the same patient.

According to a further embodiment, the medical device may be configured to reconstitute the at least one drug. Thus, the drug handling may comprise reconstitution of the least one drug, e.g. of at least one lyophilized drug. According to a further embodiment mixing of at least two drugs may be performed, e.g. with reconstitution of at least one lyophilized drug or without reconstitution.

According to a further embodiment, the fluid guide system (e.g. replaceable consumable) of the medical device may be replaceable. Thus, the technical effects mentioned above may be valid. The fluid guide system may be retained in a retaining space, e.g. within the same retaining space as the drug containers which are mentioned below.

According to a further embodiment, the medical device may comprise at least one electrically driven pump. Thus, the fluid transport may be established in a simple manner.

According to a further embodiment, the drug delivery device may comprise at least one electrically driven pump, optionally a positive displacement pump, e.g. a peristaltic pump. However, negative pressure pumps may be used as well, e.g. radial pump, diagonal pump, axial pump etc. Molecules of the drug may be more sensitive to tension compared to pressure. Thus, positive displacement pumps may be more appropriate to transport the molecules compared to negative pressure pumps and/or compared to pumps which may have a greater impact to the molecules.

According to a further embodiment, the medical device may comprise at least one electrically driven pump and/or the drug delivery device may comprise at least one electrically driven pump. Thus, electrical energy may be used in both devices for pumping of the drugs and/or for controlling. No storing of mechanical energy will be necessary. Moreover, a simple energy transfer may be used to transport electrical energy from the medical device to the drug delivery device, e.g. charging of at least one rechargeable battery or replacement of at least one non-rechargeable battery.

According to a third aspect, a system for handling at least one drug and for delivery of the at least one drug is provided. The system, e.g. a kit, may comprise:
a medical device for handling the least one drug, optionally a medical device according to the second aspect, and
a drug delivery device for delivering the at least one drug, optionally a drug delivery device according to any one of the embodiments mentioned above,
   wherein the medical device may comprise:
at least one interface portion of the medical device for detachable coupling of the drug delivery device,
   wherein the handling of the at least one drug may comprise at least one reconstitution procedure of the at least one drug, and
   wherein the at least one interface portion of the medical device may comprise at least one outflow fluid port which is configured to transfer the at least one drug to the drug delivery device after handling of the at least one drug, and
   wherein the drug delivery device may comprise:
a case,
at least one interface portion of the drug delivery device being part of an interface to the medical device,
   wherein the at least one interface portion of the drug delivery device may comprise at least one inflow port of a fluid guide system of the drug delivery device, and
   wherein the drug delivery device may be detachably connectable to the medical device via the at least one interface portion of the drug delivery device in order to transfer the at least one drug from the medical device to the at least one drug container via the at least one inflow port.

According to an embodiment, the system, e.g. kit, may comprise:
The medical device according to any one of the embodiments mentioned above, and/or
The drug delivery device according to any one of the embodiments mentioned above.

Thus, the medical device and the drug delivery device may be a pair of devices with closely connected functionality or with closely connected functionalities, e.g. with regard to fluid transfer and/or electrical energy transfer and/or mechanical energy transfer from the medical device to the drug delivery device. Data transfer may be directed from the medical device to the drug delivery device or vice versa. Bidirectional data transfer may also be possible between the two devices. The technical effects mentioned above for the drug delivery device and for the medical device are also valid for the system and vice versa.

According to an embodiment of the system, the medical device may comprise a retaining space. The retaining space may be configured to retain or may retain a plurality of drug containers at once. The drug containers may be covered by a cover member, optionally during the drug handling and/or drug preparation. Thus, it may be made sure that all drug containers are involved in the drug handling procedure in a proper manner.

According to an embodiment of the system, the medical device may be configured to handle the drugs in all drug containers of the plurality of drug containers during a drug handling procedure, e.g. there may be no drug container connected to a drug port of the medical device which drug is not used for drug handling. This may be different to a handling procedure that is described below in more detail and that is referred to as “made to order”.

According to an embodiment of the system, the drug delivery device may comprise a drug container or the medical device may comprise a drug container which may have a volume (capacity) that is sufficient to accommodate all the drugs that are handled during the drug handling procedure, e.g. the drugs originating from all the drug containers that are connected to the drug ports of the medical device.

Therefore, a single one drug handling procedure may be used, e.g. a drug handling procedure that is not interrupted e.g. by the delivery of drugs. Thus, the control of the drug handling may be simplified. Moreover, user input may be minimized.

The “large” drug container may be contained within the drug delivery device or within the medical device. If the drug delivery device comprises the “large container” there may be e.g. no “large” container within medical device that is able to store the whole amount of drug after drug handling. If the medical device comprises the “large container” there may be e.g. no “large” container within their drug delivery device that is able to store the whole amount of drug after drug handling. However, there may be reasons to use a “large” drug container within both devices, e.g. in order to enable a single drug handling step in the medical device without concurrent coupling to the drug delivery device and/or in order to enable the user to take the whole volume of drug with him/her in the drug delivery device thereafter.

Two application scenarios will be mentioned in more detailed in the following, e.g. “deliver whole charge at once” and/or “pit stop”.

According to an embodiment of the system, the medical device may be configured to transfer the drugs handled during the drug handling step into the drug container of the drug delivery device. The drug delivery device may comprise a drug container comprising a volume that is sufficient to accommodate all the drugs that are handled during the drug handling procedure.

The medical device may comprise or may be configured to comprise only the following types of containers: drug containers, e.g. comprising dry drugs which have to be reconstituted, a container comprising a diluent (e.g. solvent), e.g. WFI (water for injection) and/or an optional flush container (e.g. a bag).

The medical device may comprise or may be configured to comprise no drug container that is configured to accommodate all the drugs (optionally all the drug solution) after drug handling, e.g. after reconstitution and/or drug mixing.

The technical effect is a simple and small medical device, e.g. because the lack of a large drug container for e.g. mixed and/or reconstituted drugs within medical device. This may result in a “deliver whole charge at once” scenario which is described with reference to the figures in more detail below.

According to an embodiment of the system, the medical device may comprise a drug container comprising a first volume that is sufficient to accommodate all the drugs that are handled during the drug handling procedure. The medical device may be configured to dispense the drug from the drug container in at least two separate dispense procedures, e.g. on demand, to the drug delivery device. The first volume may also be a maximum filling volume.

According to an embodiment, the drug delivery device may comprise a drug container comprising a second volume, optionally a maximal filling volume. The second volume may be less than 60 percent of the first volume or less than 30 percent of the first volume.

Thus, the drug delivery device may be a small device, e.g. comfortable to wear by the patient. This system enables a “pit stop” scenario that is described below in more detail with reference to the figures. The drug handling (e.g. reconstitution and/or mixing) may involve all drugs within the medical device. However, the prepared drug solution may be dispensed on demand.

According to an embodiment of the system, the medical device may comprise a retaining space for a plurality of drug containers. The medical device may be configured to handle at least one drug in a first group of at least one drug container or of at least two drug containers of the plurality of drug containers during a first drug handling procedure, optionally on demand. The medical device may be configured to deliver the drug(s) of the first group to the drug delivery device during the first drug handling procedure. The medical device may be configured to handle at least one drug in a second group of at least one drug container or of at least two drug containers of the plurality of drug containers on demand during a second drug handling procedure after the delivery of the drugs of the first group into the body of a patient.

The (drug) container of the drug delivery device may have a second volume which is adapted to the first volume of the drug (solution) generated from the drug containers of the respective group of drug containers, e.g. the second volume may be in the range of 100 percent to 130 percent of the first volume.

The second volume may be less than the overall volume of the drug generated from the drugs within all groups of drug containers, e.g. less than 60 percent of the first volume or less than 30 percent of the overall volume.

Thus, again, the drug delivery device may be a small device, e.g. comfortable to wear by the patient. The system may enable a “made to order” scenario of drug handling (e.g. reconstitute and/or mix on demand) and drug transfer (e.g. on same demand). The technical effect or advantage may be that less time for handling of the drug in the solution may be necessary since not all of the drugs have to be handled at once. Moreover, there may be less time for the handled drug solution (e.g. comprising reconstituted drugs and/or mixture of different drugs) to degenerate, e.g. the drug solution may be always “fresh” because it is prepared only on demand and/or in an amount that is actually needed at the moment.

According to an embodiment of the system, the medical device may comprise:
A first receiving and sending unit configured to communicate with a cloud based platform via a first transmission protocol, and/or
A second receiving and sending unit configured to communicate with the drug delivery device via a second transmission protocol that is different from the first transmission protocol.

According to an embodiment of the system, the medical device may be configured to receive data related to the handling of the at least one drug via the first receiving and sending unit from a cloud based platform. According to an embodiment of the system, the medical device may be configured to send data related to the handling of the at least one drug via the first receiving and sending unit to the cloud based platform.

According to an embodiment of the system, data related to the delivery of the at least one drug may be received in the medical device from the drug delivery device via the second receiving and sending unit. According to an embodiment of the system, data related to the delivery of the at least one drug may be sent from the medical device via the second receiving and sending unit to the drug delivery device.

The first transmission protocol may be e.g. a cellular (mobile) network protocol, optionally at least one of GSM (Global System Mobile) also 2G (generation), UMTS (Universal Mobile Telecommunications System) also 3G, LTE (Long Term Evolution) also 4G, 5G, etc. and/or WiFi (Wireless Fidelity or W-LAN (Local Area Network), wireless LAN), e.g. in combination with the internet, e.g. IP protocol of IETF (Internet Engineering Task Force).

The second transmission protocol may be e.g. Bluetooth (Bluetooth—Special Interest Group, SIC), a physical (wired) data transmission protocol, e.g. USB (Universal Serial Bus), NFC (Near field connection) using RFID (Radio Frequency Identification). Low energy protocols may be used as well, e.g. Bluetooth Low Energy BLE.

Thus, the medical device may be a communication hub, that relays data from and to the cloud on one side and from and to the drug delivery device on the other side. Further devices may be incorporated as well, e.g. monitor device(s) which monitor at least one physiological parameter of the patient to whom the at least one drug is delivered.

According to a further or fourth aspect, a method for drug handling and/or drug delivery is provided. The method may optionally use a drug delivery device according to any one of the embodiments mentioned above. The method may optionally use a medical device according to the embodiments mentioned above or a system according to any one of the embodiments mentioned above.

The method may comprise:
Optionally, coupling a drug delivery device to a medical device, e.g. manually. The medical device may comprise a first fluid guide system for guiding at least one drug during a drug handling procedure. The drug delivery device may comprise a second fluid guide system.

Optionally, the first fluid guide system and/or the second fluid guide system may be replaceable, and/or
Prior to or after the coupling, performing the drug handling procedure of the at least one drug within the medical device, wherein optionally the drug handling may comprise reconstitution and/or mixing of at least one drug, and/or
Transferring the at least one drug from the medical device to the drug delivery device, and/or
Optionally, decoupling the drug delivery device from the medical device after transfer of the at least one drug, e.g. manually, and/or
Optionally, delivering the drug from the drug delivery device into the body of a patient. The delivering of the at least one drug to the patient may be made by intravascular infusion.

According to a further or fifth aspect, a computer-implemented method may comprise:
Sending a request to a cloud based platform for delivery of patient related data based on at least one identification of a patient, and/or
Receiving patient related data specifying details of drug handling for the patient, and/or
Handling at least one drug for the patient in at least one medical device according to the patient related data, and/or
Sending data related to the delivery of the drug to at least one drug delivery device.

Optionally, the medical device used in the computer-implemented method may be a medical device according to one of the embodiments mentioned above. The medical device may comprise a first user interface UI enabling the user to enter data and/or to receive messages related to the drug handling and/or drug delivery.

Optionally, the drug delivery device computer-implemented method may be a drug delivery device according to one of the embodiments mentioned above. The drug delivery device may comprise also a user interface UI (e.g. a second user interface) enabling the user to enter data and/or to receive messages related to the drug handling and/or drug delivery.

Thus, the computer implemented method may allow advanced reconstitution and/or mixing of drugs as well as support for the delivery of the drugs. The technical effects mentioned above for the medical device, the drug delivery device and/or the system may also apply to the computer implemented method and vice versa.

According to an embodiment, the method may comprise:
Receiving data related to the delivery of the at least one drug from the at least one drug delivery device, and/or
Forwarding the data received from at least one drug delivery device or data generated based on this data to the cloud based platform.

Embodiments of the types of data to be transmitted are mentioned above.

According to an embodiment, the method may comprise:
Receiving physical data of the patient from at least one monitoring device in the medical device, wherein the physical data indicates at least one physical parameter of the patient before and/or during and/or after delivery of the drug, and/or
Optionally forwarding the physical data to the cloud based platform, and/or
Evaluate the physical data, e.g. within the medical device and/or within the cloud based platform (device), and/or.
Depending on the evaluation of the physical data continue or stop, e.g. interrupt the drug handling and/or the delivery of the drug.

Embodiments of the types of physical parameters are mentioned below in the description of the figures, e.g. heart rate HR, pulse, etc.

Thus, an alarm may be generated, e.g. for the patient and/or for HCP. According to an embodiment the delivery of the drug may be automatically interrupted or stopped. Interrupting or stopping drug handling may prevent that costly drugs are wasted in a wrong or incorrect drug handling process.

According to an embodiment, the method may comprise:
Controlling the handling and/or delivery of the at least one drug such that:
a) (“deliver whole charge at once”): drugs in at least two drug containers may be handled during drug handling and all of the handled drugs may be transferred to a drug container of the drug delivery device during drug handling, wherein optionally the medical device may not comprise a drug container for storing the handled drug(s) and comprising a sufficient volume to receive the whole volume of the drug fluid resulting from the drugs in the at least two drug containers or in all drug containers connectable to the medical device,
b) (“pit stop”): drugs in at least two drug containers or in all drug containers may be handled during drug handling and are transferred to a drug container within the medical device to be delivered thereafter in at least two separate steps to the drug delivery device, optionally with intermediate drug delivery, or
c) (“made to order”):
The medical device may be configured to be connected to a plurality of drug containers,
At least one drug in a first group of at least one drug container or at least two drug containers of the plurality of drug containers may be handled, optionally on demand during a first drug handling procedure,
The drug(s) of the first group may be delivered to the drug delivery device, and
Thereafter, at least one drug in a second group of at least one drug container or at least two drug containers of the plurality of drug containers may be handled on demand during a second drug handling procedure.

The technical effects of variants a) to c) are mentioned above. The following commands may be used:
A “start drug handling” command e.g. in order to initiate drug handling, e.g. reconstitution of the at least one drug and/or drug mixing,
A “start drug transfer” command e.g. in order to initiate transfer from the medical device to the drug delivery device,
An “end drug transfer” command in order to end transfer from the medical device to the drug delivery device, etc.

With regard to variant c) the following may be valid. The second group of drug containers may comprise other (e.g. of the same type) drug containers compared to first group of drug containers. No intermediate manual handling of drug containers, e.g. coupling to the medical device and/or decoupling from the medical device may be performed between first drug handling procedure and the second drug handling procedure. At least two, at least three, at least four drug handling procedures may be performed without an intermediate change of drug containers of the medical device.

According to a further aspect (sixth), administering of a drug or of a drug mixture is provided that was prepared using the systems/methods mentioned above, e.g. an item according to one of the previous aspects. Thus, the drug or the drugs may be administered, e.g. injected and/or infused after the drug is or after the drugs are prepared, e.g. using the drug delivery device according to any one of the embodiments mentioned above.

The drug delivery device mentioned in all of the above aspects may comprise a container, optionally a drug container. The container may comprise a drug, optionally a drug as mentioned below in the list of drugs.

In the following, some general remarks are mentioned before details of embodiments of the invention are described below. The general remarks refer to:
1. List of drugs
2. Definitions
3. Set of aspects (general)
4. Features and feature groups FG which may be applied to any embodiment

### 1. List of Drugs

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders. As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N—(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrome.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, small modular immunopharmaceuticals (SMIP), binding-domain immunoglobulin fusion proteins, camelized antibodies, and immunoglobulin single variable domains. Additional examples of antigen-binding antibody fragments are known in the art.

The term “immunoglobulin single variable domain” (ISV), interchangeably used with “single variable domain”, defines immunoglobulin molecules wherein the antigen binding site is present on, and formed by, a single immunoglobulin domain. As such, immunoglobulin single variable domains are capable of specifically binding to an epitope of the antigen without pairing with an additional immunoglobulin variable domain. The binding site of an immunoglobulin single variable domain is formed by a single heavy chain variable domain (VH domain or VHH domain) or a single light chain variable domain (VL domain). Hence, the antigen binding site of an immunoglobulin single variable domain is formed by no more than three CDRs.

An immunoglobulin single variable domain (ISV) can be a heavy chain ISV, such as a VH (derived from a conventional four-chain antibody), or VHH (derived from a heavy-chain antibody), including a camelized VH or humanized VHH. For example, the immunoglobulin single variable domain may be a (single) domain antibody, a “dAb” or dAb or a Nanobody® ISV (such as a VHH, including a humanized VHH or camelized VH) or a suitable fragment thereof. [Note: Nanobody® is a registered trademark of Ablynx N.V.]; other single variable domains, or any suitable fragment of any one thereof.

“VHH domains”, also known as VHHs, VHH antibody fragments, and VHH antibodies, have originally been described as the antigen binding immunoglobulin variable domain of “heavy chain antibodies” (i.e., of “antibodies devoid of light chains”; Hamers-Casterman et al. 1993 (Nature 363: 446-448). The term “VHH domain” has been chosen in order to distinguish these variable domains from the heavy chain variable domains that are present in conventional 4-chain antibodies (which are referred to herein as “VH domains”) and from the light chain variable domains that are present in conventional 4-chain antibodies (which are referred to herein as “VL domains”). For a further description of VHH's, reference is made to the review article by Muyldermans 2001 (Reviews in Molecular Biotechnology 74: 277-302).

For the term “dAb's” and “domain antibody”, reference is for example made to Ward et al. 1989 (Nature 341: 544), to Holt et al. 2003 (Trends Biotechnol. 21: 484); as well as to WO 2004/068820, WO 2006/030220, WO 2006/003388. It should also be noted that, although less preferred in the context of the present invention because they are not of mammalian origin, single variable domains can be derived from certain species of shark (for example, the so-called “IgNAR domains”, see for example WO 2005/18629).

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

### 2. Definitions

A “vial” (also known as a phial or flacon) may be a small glass or plastic vessel or bottle, often used to store medication in the form of liquids, powders, or capsules. Modern vials may be made of plastics such as polypropylene (PP). Vials may comprise a volume in the range of 1 ml (milliliter) to 100 ml or in the range of 5 ml to 40 ml. Vials may be closed by a rubber or plastic plug or by a septum (e.g. membrane that can be pierced through by a needle), e.g. a PTFE (polytetrafluoroethylene) septum. The vial may comprise a smaller neck portion near its opening. A crimped metal cap, e.g. aluminum may be used to secure the plug. The crimped cap may be secured at the neck portion.

A “flexible carrier” may be a flexible container made of a thin sheet or foil, e.g. plastic. The thickness may be in the range of 25 micrometer to 100 micrometer. Alternatively, the thickness may be more than 100 micrometer. Heat sealing may be used to produce the plastic carrier.

An “infusion bag” may be a flexible carrier, e.g. a plastic carrier that is used for infusion. The infusion bag may have at least one port, e.g. a Luer Lock port, a self-sealing infusion port, medication port, spiking port, IV tubing port, etc. The infusion bag may have at least two ports. An integrated hanger opening may be located at an edge of the infusion bag. Thus, the term “infusion bag” or “infusion pouch” may refer to a plastic carrier used for infusion. Gravity may be used to empty the infusion bag slowly. A clamp may be used to regulate the fluid flow out of the infusing carrier. An alternative may be an electromagnetic infusion pump, a latex balloon or other higher pressure container, e.g. higher than the atmospheric pressure, a mechanical “platen” pump, etc. All of these containers may be used in the described embodiments.

“Intravenous therapy” (abbreviated as IV therapy) may be a medical technique that administers fluids, medications and/or nutrients directly into a person's vein. The intravenous route of administration may also be used to administer medications or other medical therapy such as blood products or electrolytes to correct electrolyte imbalances. There are several IV containers, e.g. plastic carrier, plastic bottle, etc. All of these containers may be used in the described embodiments.

“Intra-arterial” infusion may be used as well, e.g. under certain circumstances. However, intravenous infusion may be preferred for medical reasons, e.g. lower blood pressure in veins compared to blood pressure in arteries. Thus, all embodiments may also refer to intra-arterial infusion.

“Lyophilization”, also known as freeze drying, may be used for preserving biological material by removing the water from the sample, which may involve first freezing the sample and then drying it, under vacuum, at very low temperatures, e.g. at a temperature in the range of −30 C (degrees Celsius, centigrade) to −50° C. or in the range of −35 to −45° C., e.g. at −40° C. or at around/about −40° C. The lyophilization process may take 10 to 75 hours to go through all of these stages. The vacuum pressure may be less than 2 Torr (about 266 Pa (Pascal)), e.g. about 0.05 Torr (6.6 Pa), or in the range between 0.5 hPa (hectopascal) and 10² hPa, i.e. 0.01 hPa.

“Lyophilized products” in biotechnology and pharmaceuticals may include injectable solutions, proteins, oligonucleotides, peptides, and vaccines.

“Reconstitution” of lyophilized drug products may be performed before it can be administered. In simple terms this involves mixing with a fixed amount of water-based liquid (i.e., diluent), optionally sterile water and waiting and/or agitating the resulting mixture until the drug product is dissolved or dispersed.

The term “disease”, as used herein, refers to an abnormal condition that affects the body of an individual. A disease is often construed as a medical condition associated with specific symptoms and signs. In humans, the term “disease” is often used more broadly to refer to any condition that causes pain, dysfunction, distress, social problems, or death to the individual afflicted, or similar problems for those in contact with the individual. In this broader sense, it sometimes includes injuries, disabilities, disorders, syndromes, infections, deviant behaviors, and atypical variations of structure and function, while in other contexts and for other purposes these may be considered distinguishable categories. Diseases usually affect individuals not only physically, but also emotionally, as contracting and living with many diseases can alter one's perspective on life, and one's personality. Examples of diseases are “infectious disease”, “respiratory disease”, “inflammatory disease”, “cancer disease” or “cancer”, “diabetes”, etc.

The term “diabetes” or “diabetes mellitus”, as used herein, refers to or describes a group of common endocrine diseases characterized by sustained high blood sugar levels. Diabetes may be due to either the pancreas not producing enough insulin, or the cells of the body becoming unresponsive to the hormone's effects. There are several types of diabetes, e.g. type 1 and type 2.

The at least one drug or medicament may be used to heal or to mitigate any disease, e.g. any disease mentioned in this application.

The term “drug handling” refers to the reconstitution and/or to the mixing of drugs, e.g. in order to prepare them for administration. Drug handling may be done e.g. immediately before administering (e.g. injecting) the drug after the drug is prepared. Thus, the time between the end of drug handling and the beginning of administration, e.g. injection, inhalation, etc. may be less than 5 hours, less than 2 hours, less than 1 hour, less than 30 minutes or less than 10 minutes.

The term “catheter” refers to a medical device comprising a thin tube, e.g. a flexible tube. The free end of the tube may have a sharp shape in order to easy insertion of the tube into a vessel, e.g. into avascular vessel. The other end of the tube may be coupled to a connection portion, e.g. to a Luer lock portion, etc. The length of the catheter may be in the range of 3 cm (centimeter) to 30 cm or in the range of 4 cm to 20 cm. The smallest outer diameter of the catheter may be in the range of 0.3 mm (millimeter) to 10 mm or in the range of 1 mm to 5 mm. The tube of the catheter may be made of metal.

### 3. First Set of Aspects (General)

In the following, a first set of aspects is disclosed. The aspects are numbered to facilitate referencing the features of one aspect in other aspects. The aspects form part of the disclosure of the present application and could be made subject to independent and/or dependent claims irrespective of what is claimed in the application currently.

Aspect 1. Drug delivery device for delivering at least one drug, comprising:
a case,
at least one interface portion being part of an interface to a medical device,
a retaining space within the case, the retaining space configured to receive a fluid guide system or comprising the fluid guide system,
   wherein the case is configured to receive or comprises at least one drug container,
   wherein the at least one interface portion comprises at least one fluidic port of the fluid guide system, and
   wherein the fluid guide system is fluidically coupled to or is configured to be fluidically coupled to the at least one drug container, and
   wherein the drug delivery device is detachably connectable to the medical device via the at least one interface portion, and
   wherein the drug delivery device is configured to transfer the at least one drug from the medical device to the at least one drug container.

According to an embodiment of aspect 1, the at least one drug container may comprise at least one flexible portion, e.g. an enclosure portion or a sidewall portion, which may define at least a portion of at least one reservoir within the drug container. The reservoir may form a chamber that is configured to receive the at least one drug, e.g. after drug handling in a medical device.

Aspect 2. The drug delivery device according to aspect 1, wherein the drug delivery device comprises a data transmission interface portion,
wherein the data transmission interface portion comprises a data receiving and sending unit which is configured to communicate with the medical device, and
wherein the drug delivery device is configured to receive at least one of the following data from the medical device:
   amount of drug/dose to be delivered,
   rate of drug delivery,
   data indicating that the drug delivery device is filled,
   data identifying a patient, e.g. comprising a/the patient identifier,
   data requesting a value of the battery level in the drug delivery device, and/or
wherein the drug delivery device is configured to send at least one of the following data to the medical device:
   confirmation data that delivery of the at least one drug has been successful,
   start time and end time of the delivery of the at least one drug,
   start time and duration of the delivery of the at least one drug,
   data indicating the actual amount of drug delivered,
   data indicating the rate of drug delivery,
   data indicating the type of drug(s) delivered,
   data identifying a patient, e.g. comprising a/the patient identifier, and/or
   data specifying the battery level, e.g. the remaining battery level in the drug delivery device, and/or
   error data indicating an error during the delivery of the at least one drug,
   data demanding filling of the at least one drug container.

According to any one of the aspects, the at least one interface portion may comprise an electric power port which is configured to receive electrical energy from the medical device. Thus, there may be at least two interface portions or at least three different interface portions. The usability of the drug delivery device may be raised by each interface portion. The electrical power port may be combined with a physical data connection port, e.g. within an USB port.

Aspect 3. The drug delivery device according to aspect 1 or 2, wherein the fluid guide system and/or the at least one drug container is/are replaceable.

Aspect 4. The drug delivery device according to any one of the preceding aspects,
wherein the case comprises a convex coupling portion which is configured to complement a shape of a concave support portion of the medical device,
wherein the convex coupling portion extends along a length that is at least half or at least three quarter the lengths of the drug delivery device, and
wherein at least one or at least two of the at least one interface portion is or are arranged on the convex coupling portion.

Aspect 5. The drug delivery device according to any one of the preceding claims,
wherein the drug delivery device is coupled to or is configured to be coupled to a catheter or to an interface to a catheter, and
wherein the drug delivery device is configured to infuse the at least one drug into a vessel of the body of a patient.

Aspect 6. The drug delivery device according to any one of the preceding aspects,
wherein the drug delivery device is configured to receive mechanical energy from the medical device,
wherein the drug delivery device comprises a storage element or is configured to comprise a storage element, and
wherein the storage element is configured to store the received mechanical energy.

Aspect 7. The drug delivery device according to aspect 6,
wherein the drug delivery device is configured to receive the mechanical energy during the transfer of the at least one drug from the medical device to the drug delivery device via the generated fluid pressure of a fluid comprising the at least one drug,
wherein the drug delivery device is configured such that the fluid pressure loads the mechanical storage element, and
wherein optionally the mechanical storage element comprises at least one mechanical spring that is configured to store the transferred energy, or
wherein the mechanical storage element is an inflatable drug container that is configured to be inflated by the pressure.

Aspect 8. The drug delivery device according to aspect 6,
wherein the drug delivery device is configured to receive the mechanical energy via a movable or rotatable solid element that is configured to be driven by a driving element in the medical device,
wherein the solid element is coupled to the storage element in order to transfer movement or rotation from the solid element to the storage element,
wherein the storage element comprises at least one spring that is configured to be biased by a movement of the solid element, and
wherein the storage element is configured as a pump or to drive a pump.

Aspect 9. A medical device for drug handling, comprising:
a fluid guide system for guiding the at least one drug during drug handling,
an electronic control unit, and
an interface portion for detachable coupling of the drug delivery device according to any one of aspects 1 to 8 to the medical device,
   wherein the electronic control unit is configured to control the fluid flow within the fluid guide system of the medical device during the handling of the at least one drug, and
   wherein optionally the handling of the at least one drug comprises at least one reconstitution procedure of the at least one drug, and/or
   wherein optionally, the at least one interface portion comprises at least one outflow fluid port which is configured to transfer the at least one drug, e.g. comprising at least one reconstituted drug, to the drug delivery device after handling of the at least one drug.

Aspect 10. A system (e.g. a kit) for handling at least one drug and for delivery of the at least one drug, comprising:
a medical device for handling the least one drug, optionally a medical device according to aspect 9, and
a drug delivery device for delivering the at least one drug, optionally the drug delivery device according to any one of aspects 1 to 8,
   wherein the medical device comprises:
at least one interface portion of the medical device for detachable coupling of the drug delivery device,
   wherein the handling of the at least one drug comprises at least one reconstitution procedure of the at least one drug, and/or
   wherein the at least one interface portion of the medical device comprises at least one outflow fluid port which is configured to transfer the at least one drug to the drug delivery device after handling of the at least one drug, and/or
   wherein the drug delivery device comprises:
a case, and/or
at least one interface portion of the drug delivery device being part of an interface to the medical device,
   wherein the at least one interface portion of the drug delivery device comprises at least one inflow port of a fluid guide system of the drug delivery device, and/or
   wherein the drug delivery device is detachably connectable to the medical device via the at least one interface portion of the drug delivery device in order to transfer the at least one drug from the medical device to the at least one drug container via the at least one inflow port.

Thus, the system (e.g. a kit) may comprise:
a medical device according to aspect 9 for handling the least one drug, and
a drug delivery device according to any one of the aspects 1 to 8 for delivering the at least one drug.

Aspect 11. The system according to aspect 10,
wherein the medical device comprises a retaining space that is configured to retain or that retains a plurality of drug containers at once, and
wherein the medical device is configured to handle the drugs in all drug containers of the plurality of drug containers during a drug handling procedure, and
wherein the drug delivery device comprises a drug container or the medical device comprises a drug container comprising a volume that is sufficient to accommodate all the drugs that are handled during the drug handling procedure.

Aspect 12. The system according to aspect 10,
wherein the medical device is configured to transfer the drugs handled during the drug handling step into the drug container of the drug delivery device, and
wherein the drug delivery device comprises a drug container comprising a volume that is sufficient to accommodate all the drugs that are handled during the drug handling procedure.

Aspect 13. The system according to aspect 10,
wherein the medical device comprises a drug container comprising a first volume that is sufficient to accommodate all the drugs that are handled during the drug handling procedure, and
wherein the medical device is configured to dispense the drug from the drug container in at least two separate dispense procedures, e.g. on demand,
wherein the drug delivery device comprises a drug container comprising a second volume, optionally a maximal filling volume, and
wherein the second volume is less than 60 percent of the first volume or less than 30 percent of the first volume.

Aspect 14. The system according to aspect 10,
wherein the medical device comprises a retaining space for a plurality of drug containers,
wherein the medical device is configured to handle at least one drug in a first group of at least one drug container or of at least two drug containers of the plurality of drug containers during a first drug handling procedure, optionally on demand,
wherein the medical device is configured to deliver the drug(s) of the first group to the drug delivery device during the first drug handling procedure, and
wherein the medical device is configured to handle at least one drug in a second group of at least one drug container or of at least two drug containers of the plurality of drug containers on demand during a second drug handling procedure after the delivery of the drugs of the first group into the body of a patient.

Aspect 15. The system according to any one of the aspects 10 to 14, comprising:
a first receiving and sending unit configured to communicate with a cloud based platform via a first transmission protocol,
a second receiving and sending unit configured to communicate with the drug delivery device via a second transmission protocol that is different from the first transmission protocol, and wherein the medical device is configured to receive data related to the handling of the at least one drug via the first receiving and sending unit from a cloud based platform and/or wherein the medical device is configured to send data related to the handling of the at least one drug via the first receiving and sending unit to the cloud based platform, and
   wherein data related to the delivery of the at least one drug is received from the drug delivery device via the second receiving and sending unit and/or wherein data related to the delivery of the at least one drug is sent via the second receiving and sending unit (RS2) to the drug delivery device.

Aspect 16. Method for drug handling and drug delivery, optionally using a drug delivery device according to any one of the aspects 1 to 8 or a medical device according to aspect 9 or a system according to any one of the aspects 11 to 15, comprising:
coupling a drug delivery device to a medical device,
   wherein the medical device comprises a first fluid guide system for guiding at least one drug during a drug handling procedure and wherein the drug delivery device comprises a second fluid guide system,
   wherein optionally the first fluid guide system and/or the second fluid guide system is or are replaceable,
prior to or after coupling, performing the drug handling procedure of the at least one drug within the medical device, wherein optionally the drug handling comprises reconstitution of at least one drug,
transferring the at least one drug from the medical device to the drug delivery device,
decoupling the drug delivery device from the medical device after transfer of the at least one drug.

Aspect 17. A computer-implemented method comprising:
sending a request to a cloud based platform for delivery of patient related data based on at least one identification of a patient,
receiving patient related data specifying details of drug handling for the patient, handling at least one drug for the patient in at least one medical device according to the patient related data, and
sending data related to the delivery of the drug to at least one drug delivery device.

Aspect 18. The method according to aspect 17, comprising:
receiving data related to the delivery of the drug from the at least one drug delivery device,
forwarding the data received from at least one drug delivery device or data generated based on this data to the cloud based platform.

Aspect 19. The method according to aspect 17 or 18, comprising:
receiving physical data of the patient from at least one monitoring device, wherein the physical data indicates at least one physical parameter of the patient before and/or during and/or after delivery of the drug,
optionally forwarding the physical data to the cloud based platform, evaluate the physical data,
depending on the evaluation of the physical data continue or stop, e.g. interrupt the delivery of the drug.

Aspect 20. The method according to any one of the claims 17 to 19, comprising: controlling the handling of the drug such that:
a) drugs in at least two drug containers are handled during drug handling and all of the handled drugs are transferred to a drug container of the drug delivery device during drug handling, wherein optionally the medical device does not comprise a drug container for storing the handled drug(s) and comprising a sufficient volume to receive the whole volume of the drug fluid resulting from the drugs in the at least two drug containers or in all drug containers connectable to the medical device,
b) drugs in at least two drug containers are or in all drug containers handled during drug handling and are transferred to a drug container within the medical device to be delivered thereafter in at least two separate steps to the drug delivery device, optionally with intermediate drug delivery, or
c) the medical device is configured to be connected to a plurality of drug containers, at least one drug in a first group of at least one drug container or at least two drug containers of the plurality of drug containers are handled optionally on demand during a first drug handling procedure,
the drug(s) of the first group are delivered to the drug delivery device, and
thereafter, at least one drug in a second group of at least one drug container or at least two drug containers of the plurality of drug containers are handled optionally on demand during a second drug handling procedure.

Aspect 21. Medical data communication device, comprising:
a first receiving and sending unit configured to communicate with a cloud based platform via a first transmission protocol,
a second receiving and sending unit configured to communicate with a drug delivery device via a second transmission protocol that is different from the first transmission protocol, and
at least one data processing unit coupled to the first receiving and sending unit and to the second receiving and sending unit,
   wherein the medical device is configured for drug handling of at least one drug under control of the at least one data processing unit, and
   wherein the medical device is configured to receive data related to the handling of the at least one drug via the first receiving and sending unit from a cloud based platform, and/or wherein the medical device is configured to send data related to the handling of the at least one drug via the first receiving and sending unit to the cloud based platform, and
   wherein data related to the delivery of the at least one drug is received from the drug delivery device via the second receiving and sending unit and/or wherein data related to the delivery of the at least one drug is sent via the second receiving and sending unit to the drug delivery device.

Features mentioned in the description, e.g. in the appended claims and/or feature groups FG may be applied to the first set of aspects and vice versa.

### 4. Features which May be Applied to any Embodiment

A list of features, grouped in sublevels and containing variations and alternatives is presented in the following:
1) FG1. (Feature group): A drug preparation subsystem as described in the Auto Recon Feature list (see second set of aspect above, section 4) consisting of a device and consumable to prepare, dilute, reconstitute and dose a drug solution from drug vials.
2) FG2. An infusion pump sub-system, consisting of
3) F2.1. (Feature) A pump to transfer fluid (drug solution, heparin, saline) to the patient via an infusion set at a set rate
4) F2.1.1 Electromechanical Peristaltic pump
5) F2.1.1.1. Linear
6) F2.1.1.2. Rotary
7) F2.1.2. Electromechanical Positive displacement pump
8) F2.1.3. Elastomeric pump
9) F2.1.3.1. No automatic active rate controlled
10) F2.1.3.2. Electronically rate controlled
11) F2.1.4. Gravity infusion
12) F2.1.4.1. No automatic active rate controlled
13) F2.1.4.2. Electronically rate controlled
14) F2.2. Single use drug solution reservoir
15) F2.2.1. The infusion reservoir size is either:
16) F2.2.1.1. Large enough to contain the full infusion dose
17) F2.2.1.2. Smaller than the full infusion dose, requiring refilling during higher volume treatment
18) F2.3. A single use consumable unit containing all the fluid paths that contact the drug & solution during pumping including
19) F2.3.1. Means to interface to the infusion pump
20) F2.3.2. Fluid connection to the infusion set
21) F2.3.3. Fluid connection with the reservoir
22) F2.3.4. Fluid connections to heparin and saline solution for delivery before and after injection and priming the lines
23) F2.4. Means of power and electrical signals being transferred from the reconstitution sub system to the infusion pump sub-system
24) F2.5. An outer casework that contains the pump, consumable and reservoir to enable easy portability and reduce the risk of damage to the reservoir during use.
25) FG3. An infusion pump subsystem as described by FG2. where the infusion pump is detachable from the rest of the system and additionally incorporates
26) F3.1. Means of wireless communication with the rest of the system via low power communication protocols such as Bluetooth, Wi-Fi (W-LAN) or NFC (Near Field Connection).
27) F3.2. A pumping specific user interface
28) F3.3. A reserve battery to provide electrical power to the unit when detached
29) F3.4. A means of fluid connection to the drug preparation sub-system that is sterile and allows easy and quick disconnection
30) F3.4.1. This connection may be used once or multiple times
31) F3.4.1.1 Single use connection—used to transfer the full drug solution dose from the drug preparation subsystem to the pumping subsystem
32) F3.4.1.2. Alternatively, a means for this connection to be reusable between during a treatment to allow multiple connection and disconnections of the subsystems, to enable one or several of: F3.4.1.2.1. the pump subsystem reservoir volume to be less than the full infusion dose, reducing the pump subsystem size and requiring multiple refills from the drug preparation subsystem
33) F3.4.1.2.2. post IV infusion flush with a saline or sterile fluid
34) F3.4.1.2.3. pumping of pre- and post-infusion drugs such as those to prevent clotting
35) F3.4.2. The connection could take multiple forms
36) F3.4.2.1. Luer lock, manually connected by the user
37) F3.4.2.2. Septum and static needle
38) F3.4.2.2.1. Connected automatically by a mechanism when user places the pumping subsystem onto the drug preparation subsystem
39) F3.4.2.2.2. Connected automatically by an electromechanical mechanism that moves the needle relative to the septum
40) F3.4.2.3. Septum and needle that increments on disconnection to avoid piercing the same area of the septum during multiple connections
41) F3.4.2.4. Custom self-sealing connector, featuring a seal that is opened automatically on connection and closes again automatically on disconnection
42) F.3.5. Means of transferring electrical power to the device in a way that requires minimal effort from the user including wireless inductive charging, or push pins
43) FG4. The device contains an electronic computer system to control the subsystems, operate the user interface and enable connectivity features such as:
44) F4.1. A Facility for connection with an external network such as cellular or Wi-Fi Connection to:
45) F4.1.1. Receive information, such as drug preparation inputs or drug deliver treatment regime
46) F4.1.2. Upload information, such as automatic patient record monitoring or pump progress for remote monitoring
47) F4.2. A facility for wireless connection with accessories via low power communication protocols such as Bluetooth, Wi-Fi or NFC (Near field communication).
48) F4.2.1. Vital sign monitoring devices
49) F4.2.2. Detachable pump unit as described in FG3.
50) FG5. The pumping subsystem consumable as described in feature F2.3 with features to reduce the use steps, such as:
51) F5.1. A mechanism to automate the connection of the consumable to the solution reservoir
52) F5.2. Auto sterilizing features of drug preparation subsystem to the pumping subsystem
53) FG6. An alternative infusion pumping sub-system to that described in FG2. and FG3., where the pumping subsystem is detachable from the drug preparation system, but it does not use an electromechanical pump with the aim of further reducing the size of the pumping unit. Example embodiments of this include:
54) F6.1. The pumping of the fluid by the drug preparation subsystem into the pumping subsystem pressurizes the fluid to be pumped, providing the energy for infusion pumping by either:
55) F6.1.1. Stretching an elastomeric fluid reservoir in the pumping subsystem
56) F6.1.2. The fluid moves a piston that compresses a spring
57) F6.2. The drug preparation subsystem actuates a mechanism within the pumping sub-system which stores the mechanical energy and uses it for pumping, for example:
58) F6.2.1. Rotates a gear, rotating a torsion spring
59) F6.2.2. Compressing a liner spring
60) FG7. The pumping subsystem is flexible to a variety of infusion sizes by one of the following:
61) F7.1. Fixed pump casework, sized to be able to house the largest infusion volume. Infusion bag does not fill the full casework for smaller volume doses.
62) F7.2. Variants of pump casework—several different casework sizes, a smaller case for smaller volumes and a larger case for larger volumes.
63) F7.3. Fixed pump casework that does not encapsulate the infusion reservoir. The infusion reservoir may vary but the pump size remains fixed.
64) F7.4. Fixed pump casework, sized to be able to house a fixed dose, say 100 ml. For larger infusions, the reservoir must be topped up from the base unit during infusion.

The making and using of the presently preferred embodiments are discussed in detail below. It should be appreciated, however, that the present disclosure provides many applicable concepts that can be embodied in a wide variety of specific contexts. The specific embodiments discussed are merely illustrative of specific ways to make and use the disclosed concepts, and do not limit the scope of the claims.

Moreover, same reference numerals refer to same technical features if not stated otherwise. As far as “may” is used in this application it means the possibility of doing so as well as the actual technical implementation. The present concepts of the present disclosure will be described with respect to preferred embodiments below in a more specific context namely a drug delivery device, e.g. for intravascular, especially intravenous infusion and/or a drug reconstitution medical device. The disclosed concepts may also be applied, however, to other situations and/or arrangements as well, e.g. to other combined drug handling and delivery devices, to devices with subcutaneous drug delivery, to delivery devices using highly viscous drugs, etc.

The foregoing has outlined rather broadly the features and technical advantages of embodiments of the present disclosure. Additional features and advantages of embodiments of the present disclosure will be described hereinafter, e.g. of the subject-matter of dependent claims. It should be appreciated by those skilled in the art that the conception and specific embodiments disclosed may be readily utilized as a basis for modifying or designing other structures or processes for realizing concepts which have the same or similar purposes as the concepts specifically discussed herein. It should also be recognized by those skilled in the art that equivalent constructions do not depart from the spirit and scope of the disclosure, such as defined in the appended claims.

### BRIEF DESCRIPTION OF THE DRAWINGS

For a more complete understanding of the presently disclosed concepts and the advantages thereof, reference is now made to the following description in conjunction with the accompanying drawings. The drawings are not drawn to scale. In the drawings the following is illustrated in:

FIG. 1 a reusable medical device in a state with a docked infusion pump module,

FIG. 2 an optional vital sign monitor that is wearable e.g. on the wrist of the hand,

FIG. 3 an overview over a basic embodiment, e.g. a primary embodiment,

FIG. 4 details of the medical device with the cover and the infusion pump module removed,

FIG. 5 an embodiment of a fluid guide system of the medical device, optionally in a pre-use state

FIG. 6 a further embodiment of the fluid guide system comprising a flow divider,

FIG. 7 a “Dock and Go” ecosystem, e.g. comprising further data processing units,

FIG. 8 usage scenarios of the infusion pump module according to the basic embodiment,

FIG. 9 usage scenarios of the infusion pump module according to a first alternative embodiment,

FIG. 10 usage scenarios of the infusion pump module according to a “made to order” embodiment,

FIG. 11 usage scenarios of the infusion pump module according to a second alternative embodiment, variant A,

FIG. 12 an embodiment using a spring driven plunger,

FIG. 13 an embodiment using an elastomeric pump,

FIG. 14 usage scenarios of the infusion pump module according to the second alternative embodiment, variant B, and in

FIG. 15 an embodiment with mechanical charging and re-charging of the pump using a coil spring.

### DESCRIPTION OF EXEMPLARY EMBODIMENTS

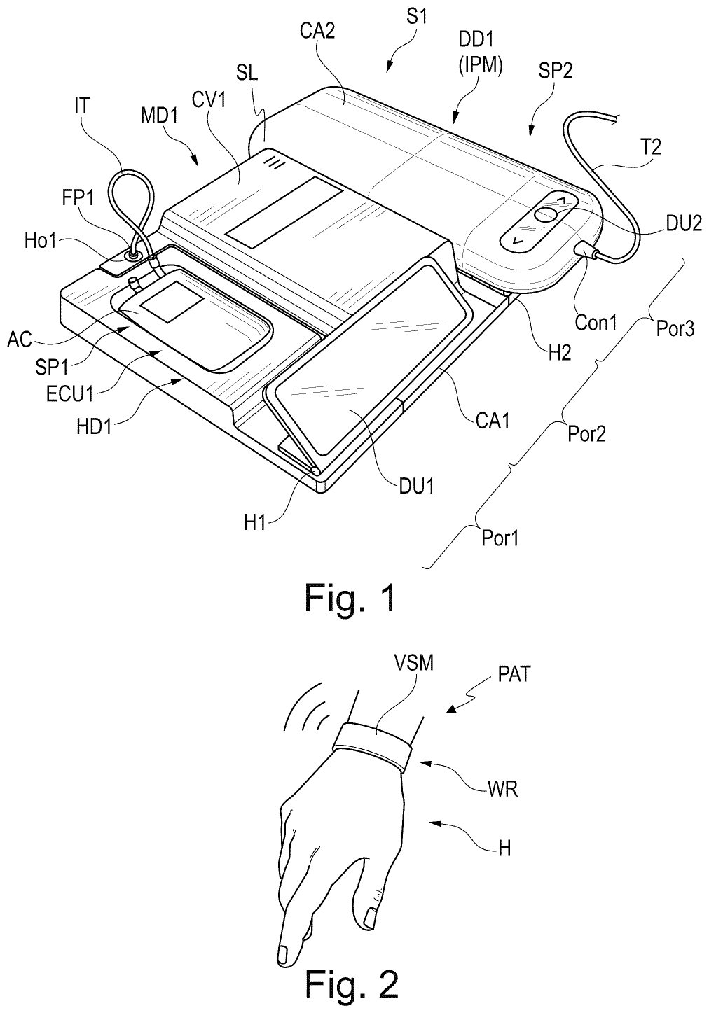

FIG. 1 illustrates a reusable medical device MD1 in a state with a docked drug delivery device DD1.

The drug delivery device DD1 may be an infusion pump module IPM, e.g. an intravenous (IV) infusion pump module. The device MD1 may be part of a system S1, e.g. a reconstitution system or other drug handling system in combination with the removable drug delivery device DD1.

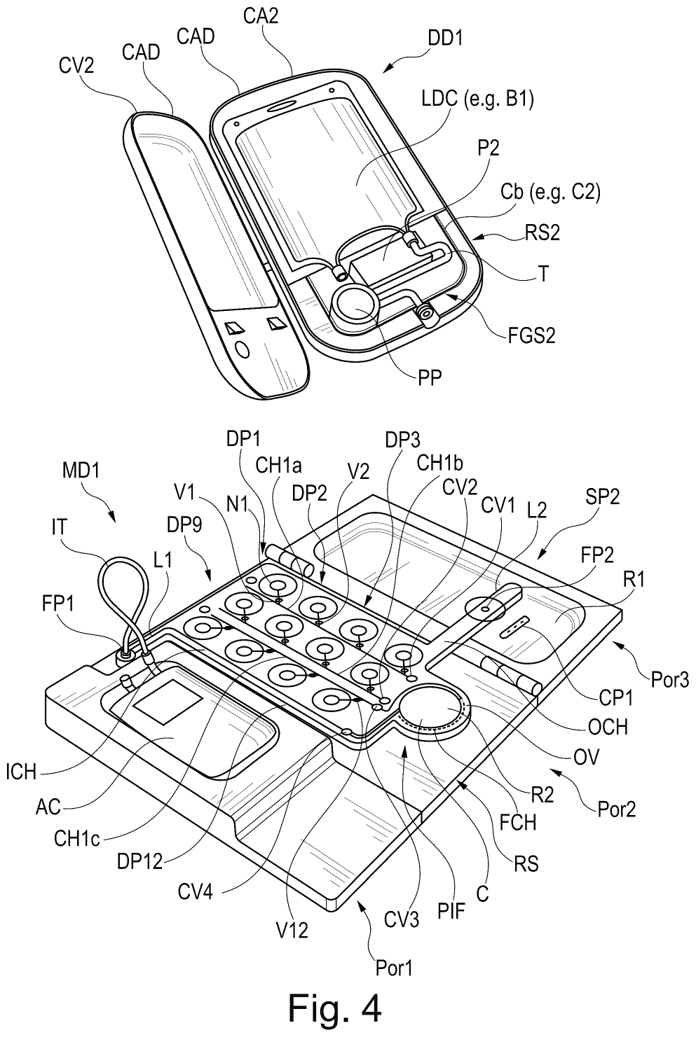

The system S1 may comprise:
The device MD1,
The drug delivery device DD1, and
A cartridge C as an implementation of a fluid guide system FGS. An embodiment of the cartridge C is illustrated in FIG. 4 and is described in more detail below with reference to FIG. 4.

The medical device MD1 may comprise an outer rigid case CA1. Further, the medical device MD1 may comprise three portions Por1 to Por3:
A left portion Por1 may comprise a fluid input portion and a user interface (UI) portion. The portion Por1 may be configured to receive an auxiliary container AC, e.g. on a support portion SP1. The left portion Por1 is described below in more detail.
A middle portion Por2 may be a receiving portion for the cartridge C (not illustrated in FIG. 1, see FIG. 4) or for a flexible carrier B. The middle portion Por2 may e.g. be covered by a cover member CV1. (Dry) drug containers (D)DC may be coupled to drug ports DP of the cartridge C or of the carrier in a prepared state of device MD1. Thus, the drug containers (D)DC may be arranged above the middle portion Por2, above the cartridge C but below the cover member CV1. The middle portion Por2 is described below in more detail.
A right portion Por3 may be a fluid output portion and may comprise e.g. a support portion SP2 that is described in more detail below. The support portion SP2 may support the drug delivery device DD1 in a docked operation state as illustrated in FIG. 1. The drug delivery device DD1 may comprise a liquid drug container LDC, e.g. a bag B1, see FIG. 4, upper part.

The left portion Por1 may be configured to receive the auxiliary container AC, e.g. a plastic carrier, optionally comprising a diluent. The diluent may be WFI (water for injection). The support portion SP1 in portion P1 of device M may allow to support the auxiliary container AC in a horizontal position, e.g. in a lying position. The shape of the support portion SP1 may be concave, e.g. in order to prevent rolling down of auxiliary container AC. Thus, the device MD1 may be shaken slightly, e.g. in order to expedite reconstitution. A first fluid port FP1 may be arranged on the cartridge C, e.g. on a “leg” thereof. The cover member CV1 may have a corresponding “leg” that extends to the left portion Por1 of the device MD1. A hole Ho1 within the “leg” of the cover member CV1 may allow passage of an inlet tube IT. The inlet tube IT may be arranged between an outflow port of the auxiliary container AC and the inlet port (FP1) of the cartridge C. Thus, a fluid may flow out of auxiliary container AC through the inlet tube IT and into the fluid port FP1.

A first electronic control unit ECU1 may be arranged e.g. in the left portion Por1. In an alternative embodiment, the first ECU1 may be arranged alternatively or additionally in at least one of the other portions Por2 and/or Por3. The first ECU1 may comprise a processor, a memory, a power unit, a data input unit and/or a data output unit.

A hub device HD1 (Com1) may be arranged e.g. in the left portion Por1. In an alternative embodiment, the hub device HD1 (Com1) may be arranged alternatively or additionally in at least one of the other portions Por2 and/or Por3. The hub device HD1 (Com1) may comprise a processor, a memory, etc.

A pivotable or otherwise movable (e.g. sliding) display unit DU1 or a fixed display unit may be arranged in portion Por1 and/or in portion Por2. The pivotable display unit DU1 may comprise a display unit DU, e.g. a display, a touch display, etc. Optional data input element(s) IE, e.g. push button(s) or a touch display may be arranged on portion Por1 and/or Por2 as well, e.g. on the display unit DU. An optional hinge H1 may allow pivoting of the pivotable display unit DU1. Alternatively, a non-movable display unit DU1 may be used.

The middle portion Por2 of the device MD1 may comprise a support portion SP/receiving space RS for the cartridge C, see FIG. 4. The (dry) drug containers (D)DC may be coupled to the cartridge C when the cartridge C is received within the support portion SP/receiving space RS. In the embodiment, e.g. 12 vials are used as (dry) drug containers (D)DC. The 12 vials may be arranged in a matrix, e.g. in three columns and four rows. Alternatively, another number of (dry) drug containers (D)DC, e.g. of vials may be used. The number of (dry) drug containers (D)DC may be in the range of 1 to 50. Moreover, another number of columns and/or another number of rows may be used.

The middle portion Por2 may comprise at least one optional output element. The output elements may be illuminated indicators, e.g. illuminated by a respective LED. Alternatively or additionally, the at least one output element may be arranged in another portion of device M, e.g. in portion Por1 and/or in portion Por3.

Thus, simple visual indicators may be used to provide the user feedback of status and/or errors. However, the visual indicators may not be used if there is a screen/display unit DU1.

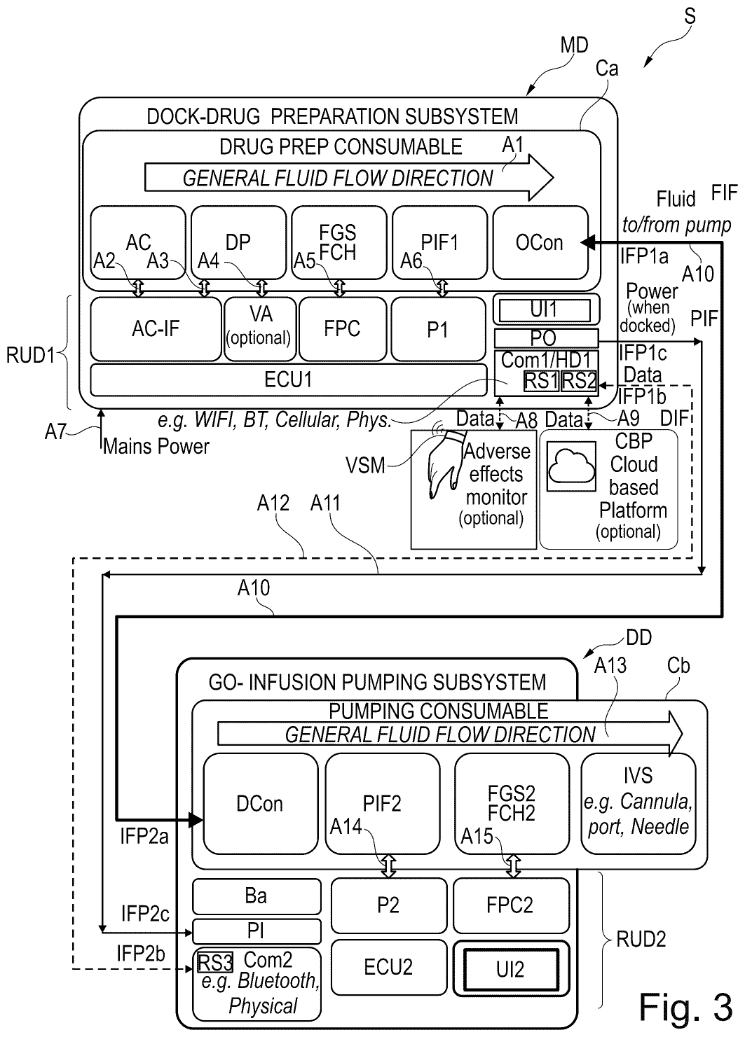

A pump P1, see e.g. FIG. 3 may be arranged within the portion Por2. Optional slots SL may allow dissipation of heat generated during operation of the pump P. The pump P1 may be arranged such that an actuator of the pump P1 engages a recess R2 within the cartridge C, e.g. a circular recess, see FIG. 4. The pump P1 may be a peristaltic pump or a pump of another type of pump.

At least one valve V, V1 may be arranged completely or partially within the portion Por2 such that it may interact with a valve portion of the cartridge C, e.g. with a flow control portion FCP of the fluid guide system FGS, e.g. of the cartridge C.

The electronic control unit ECU1 may be configured to control the pump P1 and the at least one valve V, V1 or valve portion V, V1. Moreover, the electronic control unit ECU1 may be configured to receive input from a user using the data input elements, e.g. the touch display unit DU1. Further, the electronic control unit ECU1 may be configured to signal data to the user, e.g. using the display unit DU1 and/or optional optical output elements and/or optional acoustical output elements, etc.

The right portion Por3 may comprise the support portion SP2 that may be configured to receive the drug delivery device DD1 comprising the liquid drug container LDC, e.g. an infusion bag. The support portion SP2 may have a concave surface in order to prevent that the drug delivery device DD1 may roll down, e.g. even if the device MD1 is shaken slightly in order to promote reconstitution of drug D within the (dry) drug containers (D)DC. Alternatively, the support portion SP2 may be arranged in another portion, e.g. in portion Por1 and/or Por2 of the device MD1. An optional hinge H2 may be used in order to allow pivoting of support portion SP2 about an axis that extends between portions Por2 and Por3. Thus, the storage room needed to store the medical device MD1 may be reduced. Alternatively, the support portion SP2 may be fix or may be slidable.

The drug delivery device DD1 may comprise:
A case CA2, e.g. having a cubic shape, especially with rounded edges,
A display unit DU2, e.g. comprising at least one input element and at least one output element, and
A liquid drug container LDC, e.g. flexible container B1 (bag) which may be arranged within the case CA2, see FIG. 4 and corresponding description below.

A tube T2 may be connected to a fluidic output port of the drug delivery device DD1. The output port may be part of a further consumable within the drug delivery device DD1. The tube T2 may be connected to the output port via a connecting portion Con1.

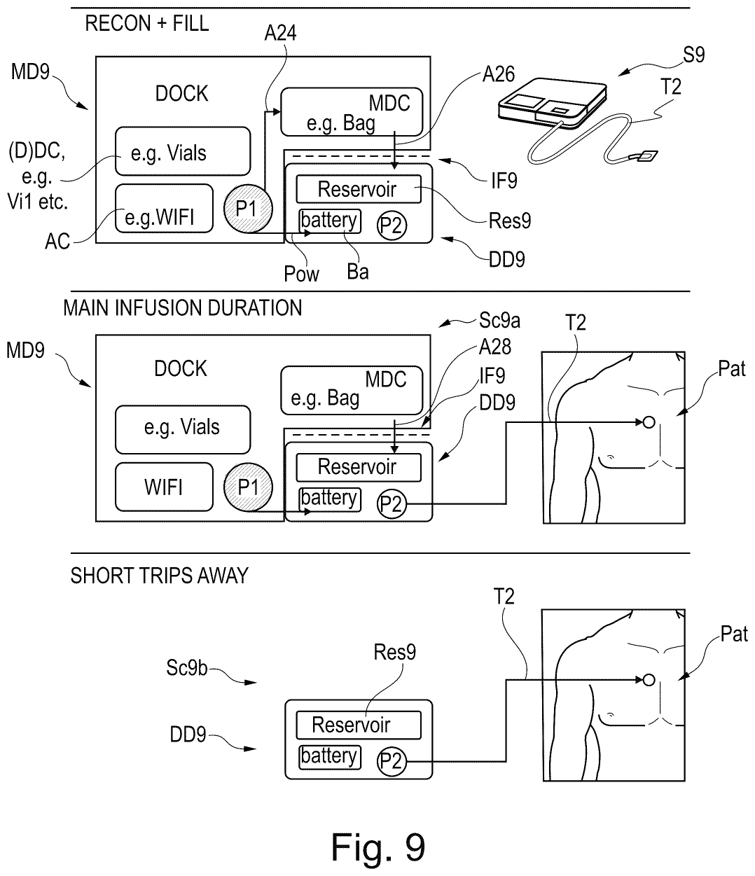

Thus, the medical device MD1 may be operated as a docking station for the drug delivery device DD1. Drug delivery device DD1 may be docked to the medical device MD1 in order to fill it with a reconstituted drug. After the reconstitution process that is performed by medical device MD1, the drug delivery device DD1 may be undocked from the medical device MD1 in order to deliver the drug D to a patient P, e.g. intravenous. Alternatively, drug delivery device DD1 may remain within the docking station during delivery of the drug D to a patient P, see e.g. FIG. 9, scenario Sc9a.

The medical device MD1 may perform the drug handling, e.g. a drug preparation. The drug handling may comprise reconstitution of drugs and/or mixing of different drugs.

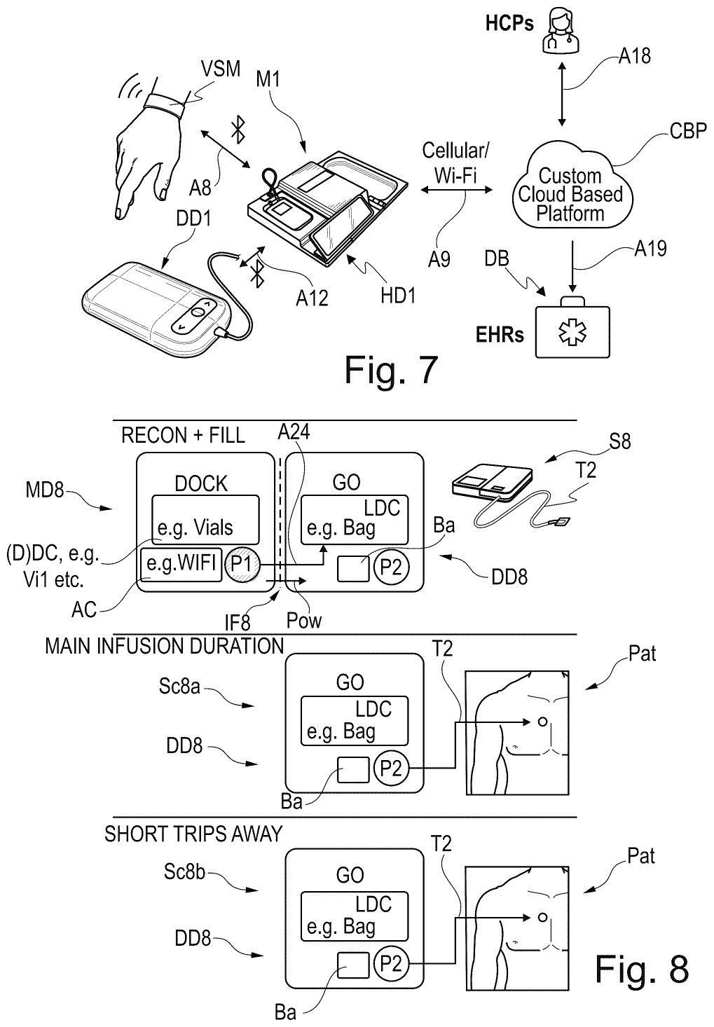

The medical device MD1, may comprise a hub device HD1/Com1, especially a hub device HD1 that may communicate with a cloud based platform CBP, e.g. via a cellular mobile network and/or via Wi-Fi or another appropriate protocol, see FIG. 7 and corresponding description as mentioned below. The hub device HD1 may receive data, e.g. from a cloud based platform CBP device. The received data may be processed by the electronic control unit ECU1 of the medical device MD1, e.g. for drug handling. Moreover, the hub device HD1 may receive digital data from the electronic control unit ECU1 of the medical device MD1, e.g. related to the drug handling process and/or from the drug delivery device DD1. Data coming from the electronic control unit ECU1 may be sent to the drug delivery device DD1 and/or to the cloud based platform CBP.

As mentioned, the hub device HD1 may also communicate with the drug delivery device DD1. Thus, the drug delivery device DD1 may be small and/or simple because it needs only a communication interface (e.g. Bluetooth (may be a trademark)) to the hub device HD1 but not to the cloud based platform CBP.

Moreover, optionally, hub device HD1 may fulfill a hub function, i.e. relaying data, for further devices, e.g. for a monitoring device, e.g. VSM, see FIG. 2, that monitors physiological parameters of the patient Pat.

Further with reference to FIGS. 1 and 4, a drug delivery device DD1 for delivering at least one drug D is described, comprising:
a case CAD, and/or
at least one interface portion IFP2 being part of an interface IF to a medical device MD1, and/or
a retaining space RS2 within the case CAD, the retaining space RS2 may be configured to receive a fluid guide system FGS2 or comprising the fluid guide system FGS2.

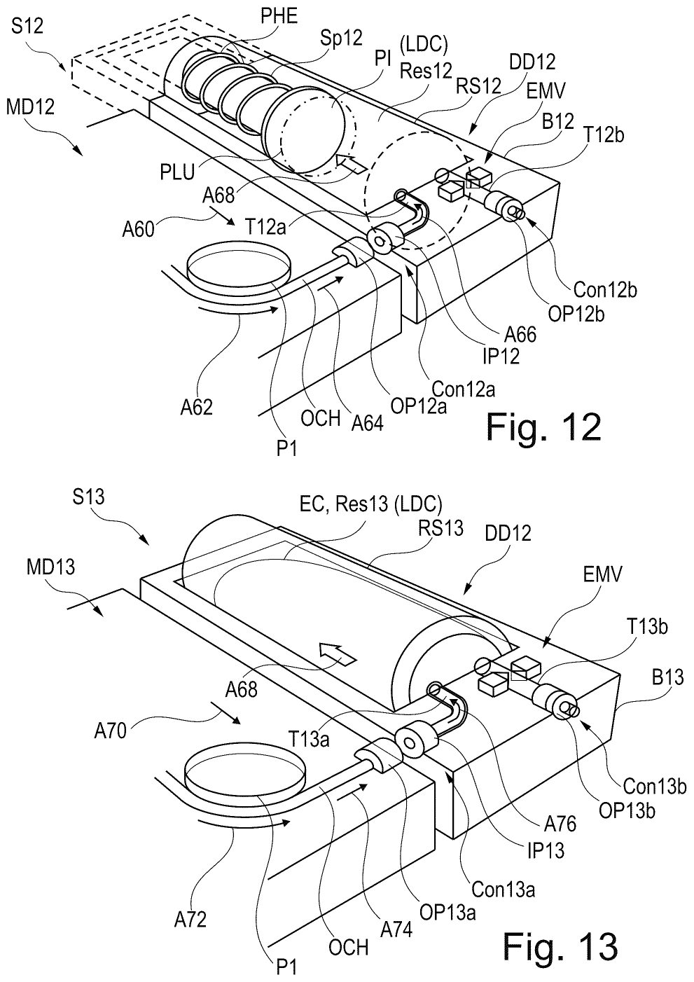

The case CAD may be configured to receive or may comprise at least one drug container LDC, Res9. The at least one interface portion IFP2 may comprise at least one fluidic port IP12, IP13 of the fluid guide system FGS2, see FIGS. 12 and 13. The fluid guide system FGS2 may be fluidically coupled to or may be configured to be fluidically coupled to the at least one drug container LDC. The drug delivery device DD1 may be detachably connectable to the medical device MD1 via the at least one interface portion IFP2, e.g. in order to transfer the at least one drug D from the medical device MD1 to the at least one drug container LDC.

The drug delivery device DD1 may be coupled to or may be configured to be coupled to a catheter or to an interface to a catheter, e.g. via tube T2. The drug delivery device DD1 may be configured to infuse the at least one drug D into a vessel of the body B of a patient Pat.

FIG. 2 illustrates an optional vital sign monitor device VSM that is wearable e.g. on the wrist WR of the hand H of the patient Pat.

The vital sign monitor device VSM may be arranged within a wrist band. Alternatively, the vital sign monitor device VSM may be comprised within a smart watch or other wearable device.

The vital sign monitor device VSM may detect and/or measure at least one, several or all of the following physical parameters of a patient Pat who carries the monitor device VSM e.g. on his/her hand:
Blood pressure (BP), and/or
Blood oxygen content, and/or
Pulse P, and/or
Heart rate HR, and/or
Heart rate variability HRV, and/or
Skin conductance, etc.

Data comprising the detected or measured physiological parameters may be evaluated in the monitoring device VSM and/or in the medical device MD1 and/or in the cloud based platform CBP or elsewhere. Thus, monitoring device VSM may be used to monitor adverse effects of drug delivery to the patient Pat, e.g. during and/or after drug delivery. In an embodiment, the patient Pat is also monitored before injection, e.g. in order to make sure that he or she is in a good physical condition for getting the drug administration, e.g. the infusion.

FIG. 3 illustrates an overview over a basic embodiment, e.g. a primary embodiment.

A medical system S may comprise:
A preparation medical device MD, and
A drug delivery device DD.

The components of the medical system S are explained in more detail in the following. Arrows A1 to A15 symbolize e.g. fluid flow and/or data flow between the components of the medical system S. In the docked state, the length of arrows A10 to A12 is exaggerated in order to have enough space for illustrating the blocks/components of the drug preparation device MD, DPD and for illustrating blocks/components of the drug delivery device DD within FIG. 3. In the undocked state, the arrow A12 may symbolize, e.g. for the case of using a wireless connection a data transfer between medical device MD and the drug delivery device DD, e.g. a transfer of a status messages, error messages, etc.

A replaceable or removable consumable (portion) Ca, e.g. comprising the cartridge C may be used within the medical device MD, e.g. a fluid guide system FGS as described below in more detail. The consumable Ca may be placed within a retaining and/or receiving space RS of the medical device MD, see FIG. 4. The medical device MD may comprise a reusable device (portion) RUD1.

The consumable Ca may comprise:
At least on auxiliary container AC, e.g. comprising a diluent that is used for reconstitution of dry drugs D or for other purposes, and/or
At least two drug ports DP, e.g. for coupling drug containers DC, optionally vials Vi1, etc. to the fluid guide system FGS, and/or
The fluid guide system FGS comprising at least one or at least two fluid channels FCH (fluid paths), e.g. cartridge C, and/or
At least one pump interface PIF1, e.g. for interfacing with a peristaltic pump P1 or other pump P1, and/or
At least one (fluidic) output connection OCon, see FIG. 4, portion Por3.

The arrow A1 indicates the general direction of the fluid flow, e.g. from the auxiliary container AC to the drug containers DC, e.g. vials Vi1 etc. and then to the output connection OCon. The pump interface PIF1 may be arranged at an appropriate location within the fluid path(s) as is described exemplary below.

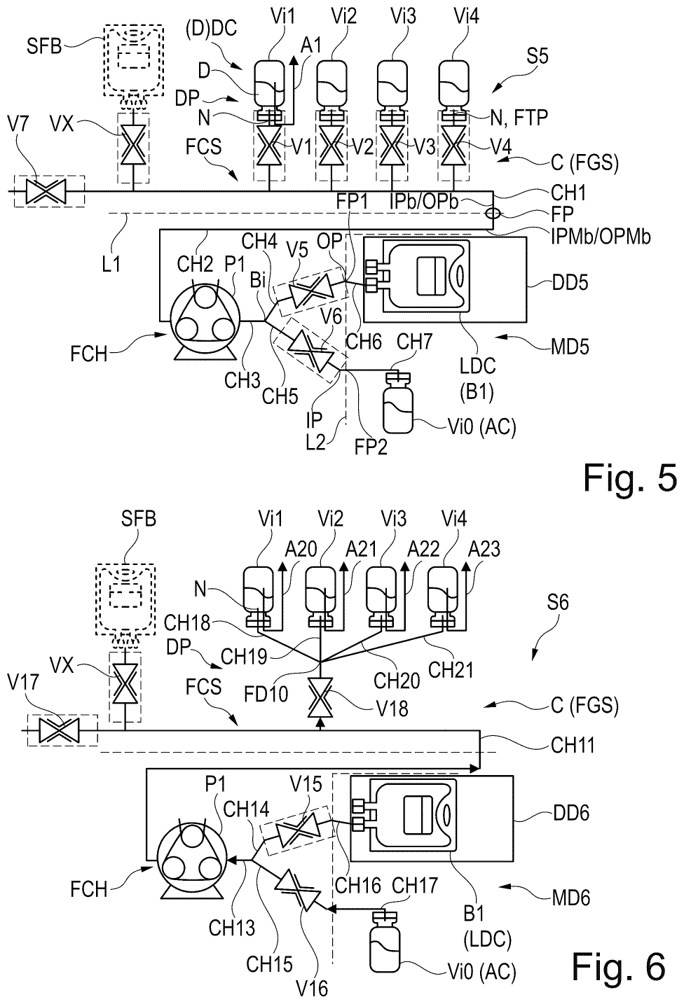

The reusable device RUD1 may comprise:
An auxiliary container interface AC-IF, e.g. the support portion SP1 and/or input tubing IT. Arrows A2 and A3 indicate the interaction between the auxiliary container interface AC-IF and the auxiliary container AC and/or the vials Vi1, etc. or other drug containers DC. The auxiliary container interface AC-IF may comprise the diluent interface and/or a smart label reader, e.g. for reading smart labels on the auxiliary container AC and/or on the vials, and/or
An (optional) vial agitation unit VA, e.g. for generating heat and/or for shaking the vials Vi1, etc. during reconstitution, see arrow A4, and/or
At least one fluid path controller FPC, e.g. valves and/or valve actuators which are coupled to the fluid guide system FGS in order to direct the fluid flow as is mentioned below in more detail, see e.g. FIGS. 5 and 6 and corresponding description, see arrow A5, and/or
A pump P1 and optional power electronics for driving the pump P1. The pump P1 may interact with the pump interface PIF1 in order to transfer a pumping motion from the pump P1 to the fluid guide system, e.g. at a flexible (tube) portion of the fluid guide system FGS. An arrow A6 indicates the interaction between the pump P1 and the pump interface PIF1.

Moreover, the reusable device RUD1 may comprise:
An electronic control unit ECU1, e.g. a central computer. The electronic control unit ECU1 may comprise e.g. a controller (microprocessor or microcontroller) and at least one digital memory, and/or
A user interface UI1, e.g. comprising a display unit DU, e.g. the display unit DU1 as mentioned above. The user interface UI1 may be coupled to the electronic control unit ECU1, e.g. in order to provide output to the user (e.g. patient Pat) and/or to enable input of the user, and/or
A power output (unit) PO, e.g. delivering electrical power Pow to the drug delivery device DD, and/or
A communication device Com1, e.g. a hub device HD1, e.g. comprising a first receiving and sending unit RS1 (e.g. cellular) and/or comprising a second receiving and sending unit RS2 (e.g. physical (wired) or WiFi/W-LAN).

The first receiving and sending unit RS1 may transmit data according to a first data transmission protocol, e.g. cellular (mobile) network protocol GSM (Global System Mobile) also 2G (second generation), UMTS (Universal Mobile Telecommunications System) also 3G, LTE (Long Term Evolution) also 4G, 5G, etc.) and/or WiFi (W-LAN (Local Area Network), wireless LAN). The first receiving and sending unit RS1 may be used by the hub device HD1 to communicate with the cloud based platform CBP.

The second receiving and sending unit RS2 may transmit data according to a second data transmission protocol, e.g. Bluetooth, physical (wired), e.g. USB (Universal Serial Bus). The second receiving and sending unit may be used by the hub device HD1 to communicate with the monitor device VSM and/or with the drug delivery device DD. There may be a physical connection, e.g. via USB between the medical device MD (first receiving and sending unit) and the drug delivery device DD, see arrow A12 (DIF—Data InterFace).

The medical system S may further comprise the vital sign monitor VSM (adverse effect monitor device) and the cloud based platform CBP.

The arrow A7 symbolizes the power connection of the medical device MD to the mains, e.g. to the utility network of an energy provider. Optionally, medical device MD may comprise a rechargeable battery to allow operation without a permanent connection to the utility network.

The vital sign monitor (device) VSM is described above, see FIG. 2. The arrow A8 indicates bidirectional data transfer between the communication device Com1 (HD1 hub device) and the vital sign monitor (device) VSM.

The cloud based platform CBP is described below in more detail, see FIG. 7. The arrow A9 indicates bidirectional data transfer between the communication device Com1 (HD1 hub device) and the cloud based platform CBP.

Moreover there may be data transfer, e.g. bidirectional data transfer between the electronic control unit ECU1 and the communication device Com1 (HD1 hub device), e.g. in order to send drug handling related data which was received via receiving and sending unit RS1, e.g. from the cloud based platform CBP to the electronic control unit ECU1. Alternatively or additionally, drug handling related data may be sent by the electronic control unit ECU1 to the receiving and sending unit RS1 for forwarding to the cloud based platform CBP.

A replaceable or removable consumable (portion) Cb, e.g. comprising a cartridge C2 may be used within the drug delivery device DD, e.g. a fluid guide system FGS2 as described below in more detail. The consumable Cb may be placed within a retaining and/or receiving space RS2 of the drug delivery device DD. The drug delivery device DD may comprise a reusable device (portion) RUD2.

The consumable Cb may comprise:
A dock connection DCon, e.g. comprising a port access element configured to be coupled to fluidic port FP2, see FIG. 4. The port access element may be arranged directly in the bottom side of liquid drug container LDC (e.g. B1) or it may be coupled to one elongated port of the liquid drug container LDC (e.g. B1), and/or
An optional pump interface PIF2, e.g. a recess allowing contact of tubing T of the cartridge to a pump P2, e.g. a peristaltic pump of the drug delivery device DD, and/or
A fluid guide system FGS2 comprising at least one fluid channel(s)/path(s) FCH2, e.g. a cartridge C2 and/or other tubing, e.g. T12a, T12b, see FIG. 12 or tubing T13a and T13b, see FIG. 13, and/or
A patients IV (intravenous) set IVS, e.g. comprising a cannula of a catheter and/or a port of the catheter and/or a needle, e.g. a needle with a flashback chamber for allowing backflow of blood indicating that the needle and/or the catheter has successfully entered the blood vessel. The needle may be configured to be inserted into the cannula of a catheter before insertion of the catheter and/or of the needle into a blood vessel or other cavity of the human body or animal body. If the catheter has been set properly the needle may be retracted out of the cannula and/or disposed. A plastic cap or other connection may be connected to the catheter which may remain for several infusions within the blood vessel of the patient.

The liquid drug container LDC (e.g. B1) may be comprised within the fluid guide system FGS2, e.g. as an integrated part, e.g. in the case of FIGS. 12 and 13 or as a part that is produced separately from the cartridge or from other tubing of the fluid guide system FGS2, e.g. in the case of FIG. 4, upper half.

The reusable part RUD2 of the drug delivery device DD may comprise:
An optional battery Ba (e.g. rechargeable or non-rechargeable), and/or
A power input PI, e.g. a plug or a socket. Alternatively or additionally, wireless charging may be used, and/or
A communication device Com2, e.g. comprising a receiving and sending unit RS3, and/or
An optional pump P2. Alternatively or additionally, the pumping function may be realized using a mechanism comprising a plunger PI and a spring Sp12, see FIG. 12 or using an elastomeric liquid drug container LDC as described below in more detail, see FIG. 13, and/or
An optional electronic control unit ECU2, e.g. a controller. The controller may comprise a microprocessor or a microcontroller. The electronic control unit ECU2 may comprise digital memory, e.g. volatile storing memory, e.g. RAM (random access memory) and/or non-volatile storing memory, e.g. EEPROM (electrically erasable programmable read only memory), especially a flash EEPROM, and/or
Fluid path controllers FPC2, e.g. valves and/or valve activators that may interact on the fluid guide system FGS2, especially on (e.g. flexible) tubing of the fluid guide system FGS2, and/or
An optional user interface UI2, e.g. a simple user interface if compared with user interface UI1.

The arrow A10 indicates of fluid interface FIF. The fluid interface FIF may comprise:
An interface portion IFP1a on the side of the medical device MD, and
An interface portion IFP2a on the side of the of the drug delivery device DD.

The arrow A11 indicates an optional power interface PIF. The power interface PIF may comprise:
An interface portion IFP1c on the side of the medical device MD, and
an interface portion IFP2c on the side of the drug delivery device DD.

The arrow A12 indicates an optional data interface DIF. The data interface DIF may comprise:
An interface portion IFP1b on the side of the medical device MD, and
An interface portion IFP2b on the side of the drug delivery device DD.

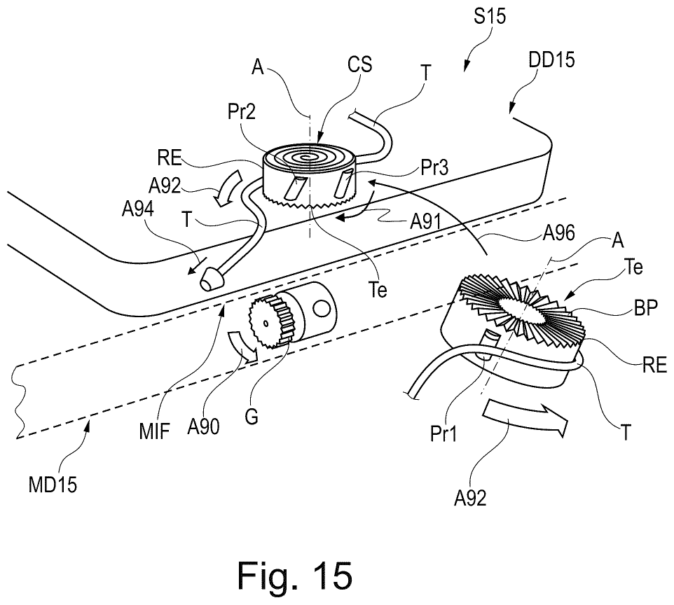

The arrow A14 indicates mechanical coupling of the optional pump P2 to pump interface PIF2. Pump interface P12 may be part of consumable (set) Cb. Thus, fluid may not come into contact with pump P2 which may be reusable, see e.g. FIGS. 12 and 15.

The arrow A15 indicates mechanical contact between fluid path controllers/actuators FPC2 and the fluid guide system FGS2, especially fluid channels FCH2 thereof. Thus, again there may be no contact between the fluid and the reusable parts of the drug delivery device DD.

Further with reference to FIG. 3, the drug delivery device DD1 may comprise a data transmission interface portion IFP2b. The data transmission interface portion IFP2b may comprise a data receiving and sending unit RS3 which may be configured to communicate with the medical device MD1, The drug delivery device DD1 may be configured to receive at least one of the following data from the medical device MD1:
Amount of drug/dose to be delivered, and/or
Rate of drug delivery, and/or
Data indicating that the drug delivery device is filled, and/or
Data identifying a patient, e.g. comprising a patient identifier, and/or
Data requesting a value of the battery level in the drug delivery device.

The drug delivery device DD1 may be configured to send at least one of the following data to the medical device MD1:
Confirmation data that delivery of the at least one drug D has been successful, and/or
Start time and end time of the delivery of the at least one drug D, and/or
Start time and duration of the delivery of the at least one drug D, and/or
Data indicating the actual amount of drug delivered, and/or
Data indicating the rate of drug delivery, and/or
Data indicating the type of drug(s) delivered, and/or
Data identifying a patient, e.g. comprising a/the patient identifier and/or
Data specifying the battery level, e.g. the remaining battery level in the drug delivery device, and/or
Error data indicating an error during the delivery of the at least one drug D, and/or
Data demanding filling of the at least one drug container LDC,

According to an embodiment the data transmission interface portion may be a wireless connection that may not be part of the at least one interface portion but may instead form a separate interface.

According to another embodiment, the data transmission interface portion may be part of a physical data transmitting connection, e.g. as being part of the at least one interface portion IFP2.

The at least one interface portion IFP2 may comprise an electric power port Pow which may be configured to receive electrical energy from the medical device MD1.

The fluid guide system FGS2 and/or the at least one drug container LDC may be replaceable.

The system S1 may comprise:
a first receiving and sending unit RS1 which may be configured to communicate with a cloud based platform CBP via a first transmission protocol, and/or
a second receiving and sending unit RS2 which may be configured to communicate with the drug delivery device DD1 via a second transmission protocol that is different from the first transmission protocol.

The medical device MD1 may be configured to receive data related to the handling of the at least one drug D via the first receiving and sending unit RS1 from the cloud based platform CBP. The medical device MD1 may be configured to send data related to the handling of the at least one drug D via the first receiving and sending unit RS2 to the cloud based platform CBP.

Data related to the delivery of the at least one drug D may be received from the drug delivery device DD1 via the second receiving and sending unit RS2. Data related to the delivery of the at least one drug D may be sent via the second receiving and sending unit RS2 to the drug delivery device DD1.

FIG. 4 illustrates details of the medical device MD1 with the cover CV1 and the infusion pump module IPM (DD1) removed.

Moreover, the display unit DU1 is not shown in order to not to hide details of the cartridge C within the retaining space RS of the medical device MD1. According to a further embodiment, the display unit DU1 may also be detachable from medical device MD1, e.g. via a sliding connector or other connector.

In addition to the parts already mentioned in the description of FIG. 1, the portion Por1 may comprise an optional “leg” portion L1 of the cartridge C. The “leg” L1 may carry the fluid port FP1 which may be used to couple inlet tube IT to the cartridge C.

The portion Por2 may comprise a receiving space RS for the cartridge C. The cartridge C may comprise at least one, several or all of:
Drug port DP1 to DP12 or more or less drug ports DP, and/or
A fluid chamber FCH, and/or
Three main channel CH1a to CH1c or more or less main channels, and/or
An input channel ICH, and/or
An output channel OCH, and/or
Valve portion V1 to V12, e.g. corresponding to the numbers of drug ports DP, and/or
Optional openings OP1, OP2, e.g. for alignment, and/or venting, and/or
Optional channel valves CV1 to CV4, and/or
At least one output valve OV, and/or
Needles N, N1, . . . , and/or
A pump interface PIF (portion), and/or
A recess R2, e.g. a through hole within the pump interface (portion PIF).

Details of the cartridge C are described below.

The portion Por3 is illustrated without the drug delivery device DD1. The portion Por 3 may comprise at least one, several of or all of:
A recess R1 forming a trough or a pan, and/or
A connecting portion CP1, e.g. comprising the power interface PIF, Pow and/or a wired data interface DIF, and/or
An optional “leg” portion L2. The length of leg L2 may be longer than lengths of leg L1, e.g. by at least 50 percent of length of leg L1. The portion L2 may comprise a fluid (output) port FP2.

The fluid connection, e.g. comprising fluid port FP2 and/or the electrical connection CP1 may allow the transfer of fluid, power and/or signals. These connections may be quick and easy to be detached and to be connected.

The drug delivery device DD1 is illustrated in an opened state in the upper half of FIG. 4. The drug delivery device DD1 may comprise at least one, several of or all of:
A case CAD, e.g. CA2 of drug delivery device DD1, and/or
A lower portion LP2 of a case CAD, and/or
A cover CV2 (portion) of the case CAD, e.g. hinged to the lower portion LP2, and/or
A liquid drug container LDC (e.g. a bag B1), especially a removable and/or replaceable drug container LDC, and/or
A consumable Cb, see FIGS. 3 and 4, e.g. a cartridge C2 or separate tubing T forming the fluid guide system FGS2, and/or
A retaining space RS2 for the consumable Cb, and/or
A port portion(s) PP, e.g. for establishing a fluid connection between the fluid port FP2 and the drug container LDC and/or the fluid guide system FGS2, and/or
The optional pump P2 or another arrangement for generating a force for drug delivery.

Thus, cartridge C2 or other tubing T may be replaceable. This may provide a sterile fluid guide system FGS2 for each application case, e.g. cross contamination, etc. will be reduced.

The liquid drug container LDC (e.g. a bag B1) may be filled with the drug solution, e.g. added to a saline solution by the drug preparation machine, e.g. by the medical device MD, MD1.

The infusion (pumping) consumable Cb may contain all the (pumping) fluid paths. The consumable Cb may be pre-sterilized and/or may be disposed after each treatment. The consumable Cb may interface to the optional infusion pump and/or to the drug preparation connection port FP2. Alternatively, gravity may be used alone or in addition to a pump in order to deliver the drug D out of liquid drug container LDC (B1).

The drug delivery device DD1 may comprise further optional units and/or elements that are not illustrated in FIG. 4, e.g. an electronic control unit ECU2, an optional receiving and sending unit RS3, etc., see description of FIGS. 1 to 3 above.

### Details of the Cartridge C:

FIG. 4 illustrates a perspective view to an embodiment of the fluid guide system FGS, e.g. to the cartridge C. The cartridge C may be pre-sterilized and/or may be disposed after each treatment. The fluid channel system FCS of the cartridge C may have the following general structure:
Drug ports DP1 to DP12 may be arranged in a matrix or in another appropriate manner. Each drug port DP1 to DP12 may comprise a needle N, N1, . . . . The needles N, N1, etc. may be stationary or movable linearly. As already mentioned, more or less than 12 drug ports DP may be used per cartridge C, and/or
Three main channels CH1a to CH1c, e.g. straight channels may be arranged parallel to each other and/or parallel to the longitudinal axis LA of the cartridge C. Alternatively, less or more than three main channels CH1a to CH1c may be used. Each main channel CH1a to CH1c may be fluidically connected to a number of drug ports DP1 to DP12, e.g. to four of the drug ports DP1 to DP12 as is described in more detail below. Each channel CH1a to CH1b may correspond to channel CH1 mentioned below, see e.g. FIGS. 5 and 6, and/or
A fluid chamber FCH forming a pump interface PIF, e.g. a flexible tube configured to interact with pump P (not part of the cartridge), e.g. a peristaltic pump P or another appropriate pump P, and/or
An input channel ICH, optionally a straight channel or a channel comprising a straight main portion, e.g. extending parallel to a longitudinal axis LA, and/or
An output channel OCH, e.g. extending perpendicular to the longitudinal axis LA within leg L2,
Valve portion V1 to V12, e.g. fluid control portions FCP, and/or
Channel valves CV1 to CV4 or valve portions CV1 to CV4 which may be arranged fluidically at end of the main channels CH1a to CH1b and at the end of the input channel ICH, respectively, and/or
An output valve OV, e.g. arranged at the outflow end of the output channel OCH.

Moreover, the following of cartridge C is illustrated in FIG. 4:
The fluidic port FP1, e.g. enabling a fluidic and/or mechanical connection to the auxiliary container AC, e.g. the diluent,
The port FP2, e.g. enabling a fluidic and/or mechanical connected to the liquid drug container LDC of the drug delivery device DD1.

The drug ports DP which are fluidically connected to one of the main channels CH1a to CH1c may form a column of the matrix Ma of the drug ports DP, e.g. DP1 to DP12. In the illustrated embodiment, four drug ports DP are used per channel CH1a to CH1c, e.g. in one column. The number of drug ports DP in one column of the matrix Ma may determine the number of lines in the matrix Ma, e.g. four lines in the embodiment. Thus, there is a 3 by 4 matrix Ma arrangement of drug ports DP and/or of (dry) drug containers (D)DC, e.g. of dry drug containers DDC. Vials may be used as dry drug containers (D)DC. Alternatively, less than four or more than four drug ports DP may be used per column of the matrix Ma. Alternatively or additionally, less than three columns or more than three columns may be used in matrix Ma. Further, alternatively other arrangements of the drug ports may be used, e.g. circular, linear, etc.

The drug ports DP of one column may be connected to the main channels CH1a to CH1c by short auxiliary channels, e.g. straight channels. The auxiliary channels and the respective main channel CH1a to CH1c may form one side of a fishbone structure, e.g. an acute angle between the respective auxiliary channel and the main channel may be in the range of 30 to 80 degrees. Alternatively, e.g. a perpendicular (about 90 degrees or 90 degrees) arrangement of a main channel CH1a to CH1c and the respective short auxiliary channel may be used. The auxiliary channels may comprise the respective valve (portions) V1 to V12, see e.g. valve portion V1 in the auxiliary channel that connects needle N1 of drug port DP1 and channel CH1a, valve portion V2 in the auxiliary channel that connects the needle N of drug port DP2 and channel CH1a, etc.

Thus, there is e.g. a drug port DP1 in the upper right corner. A drug port DP2 is arranged in the same column but near to fluid chamber FCH. A drug port DP3 follows, etc. on channel CH1a. On the left side of drug port DP1, a drug port DP5 may be arranged. The drug port DP5 may be connected to channel CH1b, e.g. to the second column channel CH1b. On the left side of drug port DP5, a drug port DP9 may be arranged. Drug port DP9 may be connected to channel CH1c, e.g. to the third column channel. A drug port DP12 may be arranged in the lower left corner of the matrix Ma.

The fluid chamber FCH may comprise the pump interface PIF. The fluid chamber FCH may comprise e.g. a flexible tube configured to be pressed and released by interaction with the pump P, e.g. peristaltic pumps. The flexible tube may extend only at the location at which interaction with the pump P is possible. Alternatively, the flexible tube may extend along at least a half of a circle or along at least three quarters of a circle as illustrated.

One end (e.g. a first end) of the fluid chamber FCH may be fluidically connected to a channel that is fluidically connected to the channel valve (portion) CV4 and/or directly to the input channel CH.

The other end (e.g. a second end) of fluid chamber FCH may be fluidically connected to the channel valve (portions) CV1 to CV3, e.g. via only one channel or via separate channels. Alternatively, channel valve (portions) CV1 to CV3 may not be used because each drug port DP may have its own valve (portion) V1 to V12.

The output channel OCH may be fluidically connected to a middle portion of fluid chamber FCH or to another appropriate portion of the fluid chamber FCH. The actuator of the pump P may actuate on a portion of the fluid chamber FCH.

The cartridge C may be used to perform the methods as described below with reference to FIGS. 5 and 6. Thus, the same technical effects may apply.

In another embodiment, valve portions V1 to V12 may not be used, e.g. the drug ports DP of each column may be filled or emptied only simultaneously to each other.

The same arrangement of the fluid channel system FCS may not only be used in a rigid cartridge C but also in a corresponding flexible carrier B.

In another embodiment, the cartridge C may be slightly modified, e.g. without “extension” features (leg(s) on at least one side and/or semicircular extension). Both long side surfaces of the cartridge C may have the same length in the modified embodiment.

In another embodiment the cartridge C may comprise more than 12 drug ports or less than 12 drug ports DP. The number of drug ports may be in the range of 2 to 100 or in the range of 3 to 50 to give only two examples.

Thus, a modified cartridge C′ for only three drug ports DP may have only the drug ports DP4, DP8 and DP12 of the cartridge C. This modified cartridge C′ may be reduced in length by omitting the other drug ports DP1, etc. Leg L1 may be arranged in an angle of about 90 degrees relative to leg L2 in order to reduce the width of the modified cartridge C′ and of a modified device.

The modified cartridge C′ may be arranged in portion Por1 only. Thus, portion Por2 may be omitted in a modified medical device MD1′. The modified device MD1′ may have the portion Por3, e.g. without modification or with modifications.

Further with reference to FIG. 4, the case CA2, CAD may comprise a convex coupling portion which may be configured to complement a shape of a concave support portion SP2 of the medical device MD1. The convex coupling portion may extend along a length that is at least half or at least three quarter the lengths of the drug delivery device DD1. At least one or at least two of the at least one interface portion IFP2 may be arranged on the convex coupling portion.

A medical device MD1 for drug D handling may comprise:
A fluid guide system FGS for guiding the at least one drug D during drug handling, and/or
An electronic control unit ECU1, and/or
An interface portion for detachable coupling of the drug delivery device DD1 according to any one of embodiments to the medical device MD1.

The electronic control unit ECU1 may be configured to control the fluid flow within the fluid guide system FGS of the medical device MD1 during the handling of the at least one drug D. Optionally the handling of the at least one drug D may comprise at least one reconstitution procedure of the at least one drug D.

Optionally, the at least one interface portion may comprise at least one outflow fluid port which may be configured to transfer the at least one drug, e.g. comprising at least one reconstituted drug, to the drug delivery device after handling of the at least one drug.

A system S1 for handling at least one drug D and for delivery of the at least one drug D may comprise:
A medical device MD1 according to any one of the embodiments mentioned above for handling the least one drug D, and/or
A drug delivery device DD1 according to any one of the embodiments mentioned above for delivering the at least one drug D.

A system S1 for handling at least one drug D and for delivery of the at least one drug D may comprise:
a medical device MD1 for handling the least one drug D, and/or
a drug delivery device DD1 for delivering the at least one drug D, wherein the medical device MD1 may comprise:
at least one interface portion of the medical device MD1 for detachable coupling of the drug delivery device DD1,
   wherein the handling of the at least one drug D may comprise at least one reconstitution procedure of the at least one drug D, and
   wherein the at least one interface portion of the medical device MD1 may comprise at least one outflow fluid port FP2 which is configured to transfer the at least one drug D to the drug delivery device DD1 after handling of the at least one drug D, and
   wherein the drug delivery device DD1 may comprise:
a case CAD, and/or
at least one interface portion IFP2 of the drug delivery device DD1 being part of an interface IF to the medical device MD1,
   wherein the at least one interface portion IFP2 of the drug delivery device DD1 may comprise at least one inflow port IP12, IP13 of a fluid guide system FGS2 of the drug delivery device DD1, and/or
   wherein the drug delivery device DD1 may be detachably connectable to the medical device MD1 via the at least one interface portion IFP2 of the drug delivery device DD1 in order to transfer the at least one drug D from the medical device MD1 to the at least one drug container LDC via the at least one inflow port IP12, IP13.

FIG. 5 illustrates an embodiment of a fluid guide system FGS (e.g. comprised in cartridge C) of a medical device MD5, optionally in a pre-use state.

A system S5, may comprise:
A medical device MD5, e.g. medical device MD, MD1, etc., and
A drug delivery device DD5, e.g. device DD, DD1, etc.

The system S5 may comprise:
Fluid channels CH1 to CH7, and/or
Valves V1 to V7, and/or
Drug ports DP, e.g. four drug ports for four vials Vi0 to Vi4, and/or
A cartridge C, e.g. comprising the fluid guide system FGS.

Moreover, there may be an optional saline bag, e.g. a saline flush bag SFB. The optional saline flush bag SFB may be fluidically connectable to channel CH1 by an optional valve VX. There are several possible embodiments of the auto-reconstitution drug preparation sub-system, e.g. of the medical device MD, MD1, MD5, MD6, etc.

Lines L1, L2 illustrate two variants as explained below in more detail. The line L2 refers to an embodiment wherein the cartridge C comprises a pump interface IF but not the pump P1 itself.

The system S5 may comprise a reconstitution system. Thus, the medical device MD5 and/or the cartridge C may comprise:
A fluid guide system FGS, e.g. according to one of the embodiments described above, optionally comprising a cartridge C, see e.g. FIG. 4 or a flexible plastic carrier B, and
reusable parts of the medical device MD5, e.g. a reconstitution device or another drug handling device, e.g. a drug mixing device.

The border between device MD5 and the fluid guide system FGS, e.g. a cartridge C or a carrier B may be different in different embodiments, see e.g. only line L1 or only line L2.

In the embodiment for which line L1 is valid, the following parts may be arranged in the fluid guide system FGS, see e.g. FIG. 4:
A fluid channel CH1, and/or
Valves V1 to V4 and V7.

In the embodiment for which line L1 is valid, the following parts may be arranged in or on device MD5:
A channel CH2, e.g. connected to or connectable to the channel CH1, and/or
A pump P, e.g. a peristaltic pump or another type of pump, and/or
Channels CH3, CH4 and CH5 e.g. arranged fluidically between pump P and an auxiliary container AC and/or a liquid drug container LDC, and/or
Valves V5 and V6, e.g. connected to channels CH4 and CH5, and/or
Channels CH6 and CH7, and/or
At least one auxiliary container AC, e.g. a vial Vi0

A liquid drug container LDC, e.g. an IV infusion bag may be arranged within drug delivery device DD5. However, alternatively and or additionally, the medical device MD5 may comprise a retaining space for a drug container MDC as mentioned below (e.g. FIG. 9), e.g. a replaceable drug container MDC, e.g. not illustrated in FIG. 5.

The fluid guide system FGS may comprise only one fluid port or several fluid ports which are used as input fluid port(s) IPb and output fluid port(s) OPb, i.e. as common input/output port IP/OP. Thus, the medical device MD5 may also comprise only one common port IPMb and OPMb coupled to (fluidically and/or mechanically) the common fluid port IPb/OPb.

In the embodiment for which line L2 is valid, the following parts may be arranged in the fluid guide system FGS, see e.g. FIG. 4:
The fluid channels CH1 to CH5, and/or
All the valves V1 to V7, and/or
The pump interface PIF.

In the embodiment for which line L2 is valid, the following parts may be arranged in or on device MD5, see e.g. FIG. 4:
Channel CH6, and/or
The at least one auxiliary container AC, e.g. a vial Vi0

The liquid drug container LDC, e.g. an IV infusion bag and/or a cannel CH7 may be arranged on or within the drug delivery device DD5. As already mentioned, the drug delivery device DD5 may comprise an additional pump for infusion, e.g. a peristaltic pump or a pump of another pump type, optionally a positive displacement pump. According to an embodiment, infusion by gravity may be used, i.e. no separate pump may be necessary.

In the embodiment for which line L2 is valid, the fluid guide system FGS may comprise at least one input fluid port IP or several fluid ports IP which are used as flow input ports only. The fluid guide system FGS may comprise at least one output port OP or several output ports OP which are used as flow outputs only.

Thus, it may be preferred, that the connection between the consumable fluid path and the diluent source/liquid drug containers may be part of the consumable.

In the embodiment of system S5, four dry drug containers DDC may be used as a maximum, e.g. vials Vi1 to Vi4. The dry drug container DDC may be coupled (mechanically and/or fluidically) to a plurality of respective drug ports DP, e.g. to four drug ports in the embodiment of system S5. The drug ports DP may comprise movable needles N or stationary needles N, i.e. needles N which cannot be moved relative to a cartridge C and/or relative to device MD1 if the fluid guide system FGS is received within or on the support portion SP/receiving space RS. A different number of drug ports DP may be used as well, e.g. in the range of 1 to 50. Instead of needles N, other types of fluid transmitting portions FTP may be used as well, e.g. cannulas without sharp tips.

Moreover, each drug port DP may be fluidically coupled or couplable to a respective valve V1 to V4 or to a respective valve portion of valves V1 to V4. The valves may be pinch valves or other types of valves, e.g. similar to pinch valves produced by Clippard.

Dry drug containers (D)DC may be coupled mechanically directly to the fluid guide system FGS and indirectly to device MD5 when the fluid guide system FGS is received within or on the support portion SP/receiving space RS. Alternatively, drug containers (D)DC may be coupled mechanically directly to device MD.

Drug ports DP may comprise respective needles N. Each needle N may be fluidically connected to a respective valve V1 to V4. The other side of a respective valve V1 to V4 may be fluidically connected to channel CH1.

One end of the channel CH1 may be fluidically connected to the valve V7. The other end of the channel CH1 may be fluidically connected to the fluid port FP (variant according to line L1) or to the channel CH2 (variant according to line L2).

One end of the channel CH2 may be fluidically connected to the fluid port FP (variant according to line L1) or to the channel CH1 (variant according to line L2). The other end of channel CH1 may be fluidically connected to one end of a fluid chamber FCH or a portion which is pressed and released by pump P1. The other end of fluid chamber FCH or of the portion may be fluidically connected to one end of channel CH3.

The other end of channel CH3 may be fluidically connected to a respective end of channel CH4 and of channel CH5. Thus, there may be a bifurcation Bi of channel CH3 to channels CH4 and CH5 in one flow direction, e.g. for flow coming from the pump P via channel CH3. A merging portion of channels CH4 and CH5 may be formed at the same location at which the bifurcation Bi is arranged in the opposite flow direction, e.g. to pump P.

The other end of the channel CH4 is fluidically connected to one side of the valve V5 (or of the valve portion V5). The other side of the valve V5 (or of the valve portion V5) may be fluidically connected to the channel CH6 which may lead to the liquid drug container LDC, e.g. an infusion bag or to a plurality of liquid drug container LDC.

The other end of the channel CH5 may be fluidically connected to one side of the valve V6 (or of the valve portion V6). The other side of the valve V6 (or of the valve portion V6) may be fluidically connected to the channel CH7 which may lead to the auxiliary container AC, e.g. a flexible carrier or a vial Vi0. Alternatively, several auxiliary containers AC may be used.

Thus, the fluid channel system FCS of the fluid guide system FGS may comprise several fluid channels, e.g. needles or needle portions, channels CH1 and/or channel CH2, etc. Some of the channels may be fluidically separated from other channels in one operation mode of a respective valve V or of respective valves V. Moreover, the same channels may be fluidically connected to the same channel(s) in another operation mode of the same valves V, e.g. a channel between valve V1 and vial Vi1 relative to channel CH1, etc.

In the following the operation of system S5 is described in detail. A dashed rectangle around a valve V illustrates the closed state of this valve. If there is no dashed rectangle around a valve V, this illustrates the open state of this valve V. The valves may be actuated by electromechanical or other actuators. Driving units may drive the actuators. The driving units may be controlled by the electronic control unit ECU1.

Step St0: Coupling of the vials to the drug ports

At the beginning, the vials Vi1 to Vi4 may be coupled to the drug ports DP, e.g. fluidically via e.g. needles and/or mechanically, e.g. via mechanical elements as mentioned in the introductory part and/or below vial engagement clips VC. The coupling of the vials Vi1 to Vi4 to the drug ports DP may be done manually. At the end of step St0, all four vials Vi1 to Vi4 may be mechanically and/or fluidically coupled to the respective drug port. Thus, the following steps St1 to St6 may be performed without further coupling of at least one vial to a drug port DP and/or without decoupling vials from the drug ports DP. This may allow fast performance of the reconstitution steps or of other drug handling steps.

Step St1: Pre-use step or pre-use configuration

All valves V1 to V7 may be closed. Thus, there is no fluid connection between e.g. auxiliary container AC and channels CH1/CH2. Moreover, there may be no fluid connections between (dry) drug containers (D)DC (which may comprise lyophilized drugs D) and channel(s) CH1/CH2. Channel CH1 is not fluidically connected to the environment because valve V7 is closed. The pump P, P1 may be in a switched off state. Moreover, valve V5 may be in a closed state thereby preventing that fluid flows out of liquid drug container LDC which may be empty or which may comprise a saline solution at the beginning of the reconstitution process which is described in the following.

In the following, different operation states of drug handling using the medical device MD5 of FIG. 5 are described, e.g. priming (St2), dilution/reconstitution of a first drug container (St3), e.g. vial, of a second drug container (St4a), etc. (St4b . . . ), flushing (St5) and transfer (St6) of the reconstituted drugs to a further drug container (e.g. IV carrier).

A step St2: priming step or priming configuration.

There may be the following valve states:
Valves V1 to V4 are still closed,
Valve V5 remains closed, and
Valves V6 and V7 are open.

The pump P1 may be switched on by the electronic control unit ECU1. Thus, fluid is pumped from fluid source, e.g. diluent in auxiliary container AC, e.g. vial Vi0 through the pump P1 or through a fluid chamber FCH of pump P to prime the tubing, e.g. channels CH1 and CH2 with fluid, e.g. a liquid fluid, optionally with a diluent. Air is pushed out through valve V7 in order to vent/drain the fluid channel system FCS. The fluid flow that is used for flushing may be stopped when all air is flushed through system S5, i.e. there is no air or only small residues of air within system S5.

A step St3: Dilution/reconstitution vial Vi1 (or other drug container DC) or dilution configuration.

There may be the following valve states:
Valve V1 open,
Valves V2 to V4 are still closed,
Valve V5 remains closed,
Valve V6 remains open, and
Valve V7 is closed.

Using pump P1 (controlled by the ECU), fluid is pumped out from auxiliary container AC, e.g. vial Vi0 to drug Vial Vi1, e.g. through channels CH1 and CH2. The necessary fluid (e.g. liquid) volume may be pumped in a controlled manner (ECU) by e.g. a positive displacement pump, e.g. pump P. Air may be vented from drug container DC1, e.g. from vial Vi1 by a hydrophobic vent which may be part of the consumable to vial connection which may allow air only to freely escape/enter, see arrow A16. Alternatively, there may be a negative pressure within vials Vi1 to Vi4 or other drug containers, e.g. mitigating the venting issue.

According to another embodiment, the needle N may have two channels or two needles N may be used per vial or per other drug container DC.

If the vial or the other drug contained DC is connected to the atmosphere, a filter may be used to lower the risk of contamination, e.g. an aseptic particulate filter.

Pumping of pump P1 may be stopped at the end of step St3, e.g. when enough liquid for reconstitution is pumped into vial Vi1 or another first drug container DC.

A step St4a (similar to steps St4b to St4x): Dilution/reconstitution of vial Vi2 to x, e.g. vial Vi4.

There may be the following valve states:
Valve V1 is closed,
Valve V2 is open,
Valves V3 and V4 are still closed,
Valve V5 remains closed,
Valve V6 remains open, and
Valve V7 is closed.

Fluid may be pumped from the liquid source, e.g. the diluent source vial Vi0 to drug Vial 2 or another second drug container. The liquid volume may be pumped controlled using the ECU1 and e.g. a positive displacement pump. Air may be vented from drug vial Vi2 or another second drug container by a hydrophilic vent which may be part of the consumable to vial connection which allows air only to freely escape/enter. Thereafter, pump P1 may be stopped or switched off by the ECU.

This process may be repeated until all vials Vi1 to Vi4 or other drug containers DC are filled to an appropriate level, e.g. with the diluent.

At the end the valve of the last drug port DC may be closed, e.g. valve V4. Valve V6 may also be closed.

An optional intermediate reconstitution step is not illustrated in a separate figure. During reconstitution now fluid flows may be established, e.g. no valves or valve portions of valves V1 to V7 may be operated by the ECU. Reconstitution may last a specific time, e.g. in the range of 1 minute to 1 hour or in the range of 5 minutes to 50 minutes to give only two possible examples. Reconstitution may be supported by additional measures, e.g. as mentioned in this document and/or in order to shorten the time necessary for complete reconstitution of drug(s) D.

Thus, there may be a step where the medical device MD5 may do not anything actively, but essentially pauses to give time for the reconstitution to occur. One variant may be that the medical device MD5 repeatedly pushes fluid in and out of the vial to encourage/speed up reconstitution.

Additional measures may comprise:
Heating or warming of drug vials Vi1 to Vi4 or of other drug containers DC, and/or
Gentle shaking of medical device MD5, e.g. manually or automatically, e.g. using a shaking device available in the market, and/or
Other measures as mentioned above and/or below, etc.

A step St5: flush(ing) or flush configuration.

At the end of the reconstitution phase, the following valve states may be established by the ECU1 using the FCP, etc.:
Valves V1 to V4 may remain closed,
Valve V5 may be opened,
Valve V6 may remain closed, and
Valve V7 may be opened.

The diluent or other fluid in the line, e.g. within channels CH1 and CH2 may be optionally flushed out using e.g. liquid from the IV carrier fluid and/or from the auxiliary container AC. If the diluent or other fluid is flushed, the pump P1 may be stopped by the ECU.

Alternatively, saline flushing bag SFB may be used for flushing.

A step St6—transfer of drug solution to IV carrier or to another drug container DC.

The following valve states may be established by the ECU using the ConS1, FCP etc.:
Valve V1 may be opened,
Valves V2 to V4 may remain closed,
Valve V5 may remain open,
Valve V6 may remain closed, and
Valve V7 may be closed.

Step St6 may follow after step St5. The pump P1 may be switched on by the ECU1 and a specific number of revolutions may be used. The rotation direction may be opposite to the previously used rotation direction of pump P1. Fluid may be transferred from e.g. drug vial Vi1 (or another first drug container DC) to IV carrier or to another appropriate liquid drug container. Air may flow into the drug vials by hydrophilic vent which may be part of the consumable to vial connection which may allow air only to freely escape/enter.

Step St6 may be repeated for the other drug containers DC, e.g. drug containers Vi2 to Vi4 sequentially. At the end, all valves V1 to V7 may be closed and the pump P1 may be switched off by the ECU1. However, alternatively, pump P1 may not be switched off during switching of the valves V1 to V4.

At the end of the transfer step(s) St6, the pump P1 may be switched off by the ECU1 and/or all valves may be closed by the ECU1.

A user, e.g. HCP (health care professional) or non-HCP may remove drug delivery device DD5 and use liquid drug container LDC, e.g. for an infusion. Fluid guide system FGS may be taken out of the support portion SP/receiving space RS and may be disposed, e.g. optionally together with used drug containers DC. The auxiliary container AC may be also disposed.

The medical device MD5 may be prepared again for the following reconstitution or other drug handling process when necessary.

Similarly, the fluid guide system FGS2 of the drug delivery device DD5 may be taken out of the support portion SP/receiving space RS and may be disposed, e.g. optionally together with used drug container LDC. The drug delivery device DD5 may be prepared again for the following drug delivery process when necessary.

In other embodiments, the drug containers DC, e.g. vials Vi1 to Vi4 of system S5 may be fluidically connected in a series manner. Alternatively or additionally, a combination of parallel fluidic connection of drug containers and of series fluidic connection of drug containers

FIG. 6 illustrates a further embodiment of the fluid guide system FGS comprising a flow divider FD10.

A system S6 may comprise:
A medical device MD6, e.g. the device MD, MD1, etc. as mentioned above, and/or
A drug delivery device DD6, e.g. the device DD, DD1, etc. as mentioned above.

The medical device MD6 may comprise fluid channels CH11 to CH21, valves V15 to V18 and/or VX and a flow divider FD10. Flow divider FD10 may comprise a fluid distributor, e.g. comprising at least one bifurcation.

The drug delivery device DD6 may comprise a liquid drug container LDC, e.g. a flexible bag. Optionally the drug delivery device DD6 may comprise a pump or other force generating device for delivering of at least one drug D. However, additionally or alternatively, drug delivery of at least one drug D from drug delivery device D by gravity may be used.

The system S6 may have similar components as the system S5, e.g.:
Four drug ports DP illustrated in FIG. 6 correspond to the four drug ports DP of FIG. 5, and/or
Again, four drug containers DC, e.g. vials Vi1 to Vi4 are connected mechanically and fluidically to a respective one of the drug ports DP. A different number of drug ports DP may be used as well, e.g. in the range of 1 to 50, and/or
Channels CH11 to CH17 correspond to the channels CH1 to CH7 respectively, e.g. the channel CH17 corresponds to the channel CH7, and/or
An auxiliary container AC is fluidically connected to the channel CH17, and/or
A liquid drug container LDC, e.g. a plastic infusion bag may be fluidically connected to the channel CH6, and/or
A pump P1 may be fluidically connected between the channels CH12 and CH13, and/or
Valves V15, V16 and 17 correspond to the valves V5, V6 and V7 respectively.

However, there may be the following differences between system S6 and system S5:
Only one valve V18 may be used instead of the four valves V1 to V4. One side of the valve V18 may be fluidically connected with the channel CH11. The other side of the valve V18 may be fluidically connected with the flow divider FD10, and/or
Four channels CH18 to CH21 may be fluidically connected to the flow divider FD10 on one side. The other side of the channels CH18 to CH21 may be connected with respective needles N of the four drug ports DP.

The system S6 may have similar states St1 and St2 as described above for system S5, see FIG. 5 (pre-use state) and priming state (not illustrated but mentioned above).

In step St3 the following valve configuration may be valid:
fluid channel CH, CH 10 to CH17
Valve V16 may be in an open state,
Valves V15 and V17 may be in a closed state, and
Valve V8 may be in an open state.

The step St3, e.g. simultaneous dilution/reconstitution of all vials or other drug containers DC as illustrated in FIG. 6 may correspond to e.g. step(s) St3, see filling of the first drug container, e.g. of vial Vi1 as mentioned above and to step St4a to 4x as mentioned above (filling of the second container DC to the fourth drug container DC, e.g. of vials Vi2 to Vi4).

Thus, fluid may be pumped from a liquid source, e.g. a diluent source to all drug containers DC simultaneously and/or at the same time, e.g. to all vials Vi1 to Vi4. Thus, liquid may be pumped in parallel through all channels CH18, CH19, CH20 and CH21, e.g. to all channels between fluid divider FD10 and the drug ports DP. In other words, when fluid is pumped to a first one of the drug containers DC, e.g. vial Vi1 fluid may also be pumped to a second one (or further ones) of the drug containers DC, e.g. vial Vi2.

The number of drug ports DP may be in the range of e.g. 2 to 100. The liquid volume may be pumped under control of the ECU by the pump P1, e.g. a positive displacement pump. The fluid flow may be equally divided (e.g. split) using the flow divider FD10. Air may be vented from all drug containers DC, e.g. from all drug vials Vi1 to Vi4 simultaneously by hydrophilic vents which may be part of the consumable-to-vial-connection which may allow air only to freely escape/enter, see arrows A20 to A23.

The following sequence of operation steps may be the same as mentioned above for system S5:
An optional separate reconstitution step St, e.g. supported by further measures as mentioned in this document, e.g. mechanical agitation by a user and/or by the machine, warming of the vials, pumping fluid into and out of the vials, etc. A control system may be configured accordingly, especially in order to prevent damage to the sensitive drugs D during reconstitution and/or mixing, and/or
A step St5, i.e. flush channels CH11 and CH12 with liquid from container LDC, e.g. carrier B1 as mentioned above. Alternatively, liquid from saline (or other solution) flush bag SFB may be used, and/or
A step St6, i.e. transfer of drug solution from drug containers DC, e.g. vials Vi1 to Vi4 to IV carrier as mentioned above, i.e. to liquid drug container LDC, e.g. flexible bag B1.

Moreover, the same final steps as mentioned above may be performed also by the system S6, e.g. switching off of the pump P1, etc.

In other embodiments, the drug containers DC, e.g. vials Vi1 to Vi4 of system S6 may be fluidically connected in a series manner. Alternatively or additionally, a combination of parallel fluidic connection of drug containers and of series fluidic connection of drug containers

Further with reference to FIGS. 5 and 6 the following may be implemented: A optionally flexible inlet tube IT (e.g. channel CH7, CH17) may be fluidically connected to the auxiliary container AC, e.g. vial Vi0 or to a flexible container, e.g. to a plastic bag. The hole Ho1 may be arranged within the cover CV. Hole H1 may allow passage of the inlet tube IT from outside into the retaining space RS.

FIG. 7 illustrates a “Dock and Go” system, e.g. comprising further data processing units.

A communication network CN may comprise:
The medical device MD, MD1, etc. for preparation/handling of at least one drug D as described above, and/or
The drug delivery device DD, DD1, etc. and/or
Optionally, the vital sign monitor VSM (device), and/or
A (custom) cloud based platform CBP.

The medical device MD, MD1, etc. may operate as a hub device HD1, e.g. providing physical and/or data connections to the other devices of the communication network CN.

In detail, there may be the following connections:
The arrow A8 may correspond to the arrow A8 as illustrated in FIG. 3 and as described in the corresponding description above. Thus, the arrow A8 may be a data transfer connection between the vital sign monitor VSM and the medical device MD, MD1, e.g. a wireless connection, optionally a Bluetooth connection.
The arrow A9 may correspond to the arrow A9 as illustrated in FIG. 3 and as described in the corresponding description above. Thus, the arrow A9 may be a data transfer connection between medical device MD, MD1 and the cloud based platform, e.g. a wireless connection, optionally (cellular/Wi-Fi).
The arrow A12 may correspond to the arrow A12 as illustrated in FIG. 3 and as described in the corresponding description above. Thus, the arrow A12 may be a data transfer connection between drug delivery device DD, DD1 and the medical device MD, MD1, e.g. a wireless connection, optionally a Bluetooth or a physical data transfer connection.
The arrow A18 may indicate remote monitoring and/or troubleshooting and/or treatment setup, e.g. by health care professional(s) HCPs or other means, e.g. an A1 (artificial intelligence) system.
An arrow A19 may indicate e.g. an automatic EHR (electronic health record) update based on data send by medical device MD, MD1, etc. or by other devices of the communication network CN and/or based on input data entered by the health care professional HCP or other entity.

Moreover, the (custom) cloud based platform CBP may not only be connected to the medical device MD, MD1, etc. that is used by a first patient but it may be connected to many other medical devices that are used by other patients, e.g. to hundreds or to thousands of such devices. Usage of a distributed cloud based platform CBP is possible as well. Health care professional(s) HCP(s) may therefore efficiently support a high number of patients.

Thus, the medical device MD1 (Dock) may act as the connectivity hub, using its e.g. cellular and/or e.g. Wi-Fi (wireless fidelity, W-LAN) connection to securely transfer data to the cloud CBP and e.g. its Bluetooth connectivity to communicate with the vital signs monitor VSM and the drug delivery device DD1, e.g. comprising a pump. By not having a cellular/Wi-Fi connectivity in the drug delivery device DD1 (Go pump), it may be allowed that the drug device DD1 may be made smaller and with lower weight, e.g. without losing the connectivity benefits.

The patient Pat may not require additional connectivity devices or to use their own devices (i.e. smartphones, etc.) to connect additional accessories such as vital signs sensors, e.g. of the vital sign monitoring device VSM to the communication network CM. The custom cloud based platform CBP may store and analyze and/or evaluate the data from at least one of the drug delivery device DD1 (e.g. comprising a pump, e.g. pump P1), drug preparation in medical device MD1, vital signs monitors VSM and pumping of the at least one drug D. This may allow that these data is visible in a single place.

A database DB may be established, e.g. in the cloud based platform CBP using the transferred and/or evaluated data. The database DB of recorded data could be used to build models to predict potential adverse effects to patients Pat early and/or to reduce the severity of such effects. These models may be used to stop or interrupt drug preparation/handling, e.g. reconstitution and/or drug mixing or drug delivery if considered necessary, e.g. due to errors or other unforeseen events.

FIG. 8 illustrates usage scenarios of the infusion pump module according to the basic embodiment.

A system S8 may comprise:
A medical device MD8, e.g. similar to medical device MD, MD1, etc., and/or
A drug delivery device DD8, e.g. similar to drug delivery device DD, DD1, etc.

Thus, the medical device MD8 may comprise:
A pump P1, and/or
An auxiliary container AC (e.g. WFI), and/or
A (dry) drug container(s) (D)DC, e.g. vial Vi1 to Vi3 or Vi1 to Vi4 or Vi1 to Vi12.

Medical device MD8 may not comprise a drug container for the handled/prepared drugs.

Furthermore, the drug delivery device DD8 may comprise:
An optional pump P2, and/or
A liquid drug container LDC, e.g. a flexible bag, and/or
An optional battery Ba, e.g. a rechargeable battery Ba.

A tube T2 may be fluidically coupled to an output port of the drug delivery device DD8. An interface IF8 may be used between the medical device MD, MD1, etc. and the drug delivery device DD, DD1, DD8 etc. The interface IF8 may comprise a fluidic interface FIF and/or power interface PIF, see arrow Pow and/or other interfaces as mentioned above, e.g. a data transfer interface. The fluidic interface of the interface IF8 may be established between the fluid guide system of the medical device MD8 and the liquid drug container LDC of the drug delivery device DD8. Thus, the medical device MD8 may not comprise a liquid drug container which stores the drug(s) after drug handling, e.g. after reconstitution and/or drug mixing

A reconstitution and fill procedure may be performed in the docked state of the drug delivery device DD8 to the medical device MD8. Thus, the liquid drug container LDC may be filled directly and in one step without intermediate decoupling or undocking of the drug delivery device DD8 form the medical device MD8, see arrow A24 indicating the fluid transport or transfer between both devices MD8 and DD8.

A usage scenario SC8a may be valid for a main infusion duration wherein the drug is pumped e.g. by pump 2 into the body of the patient Pat. The drug delivery device DD8 may be undocked from the medical device MD8, i.e. in order to enable the patient Pat to go around and/or to move away from the docking station MD8. Alternatively, the main infusion may take place when the drug delivery device DD8 is docked to the medical device MD8, e.g. if the patient Pat sleeps or sits near the medical device MD8.

The liquid drug container LDC may be changed after each use, e.g. after it is again empty. The liquid drug container LDC may be changed together with the fluid guide system FGS2 of drug delivery device DD8. The fluid guide system FGS of medical device MD, MD8 may be changed after each use, e.g. after the drug handling (reconstitution and/or drug mixing) and/or before the next drug handling procedure.

A usage scenario Sc8b which may be similar to the usage scenario SC8a may be valid for short trips of the patient Pat away from the medical device MD. The patient Pat may undock the drug delivery device DD8 from the medical device MD8 an may take the drug delivery device DD8 with him or her. Thus, the drug delivery device DD8 may enhance the freedom of the patient Pat to move around during infusion.

Both scenarios SC8a and SC8b may be based on variant a) as mentioned above e.g. “deliver whole charge at once” to the drug delivery device DD8.

The fill level of the liquid drug container LDC may be detected within device DD8, e.g. using a fill sensor device and/or indirectly via detecting the time for which the pump P2 is operated. There may be an indication of the fill level on the device DD8 and/or an alarm may be generated if the LDC is empty or almost empty.

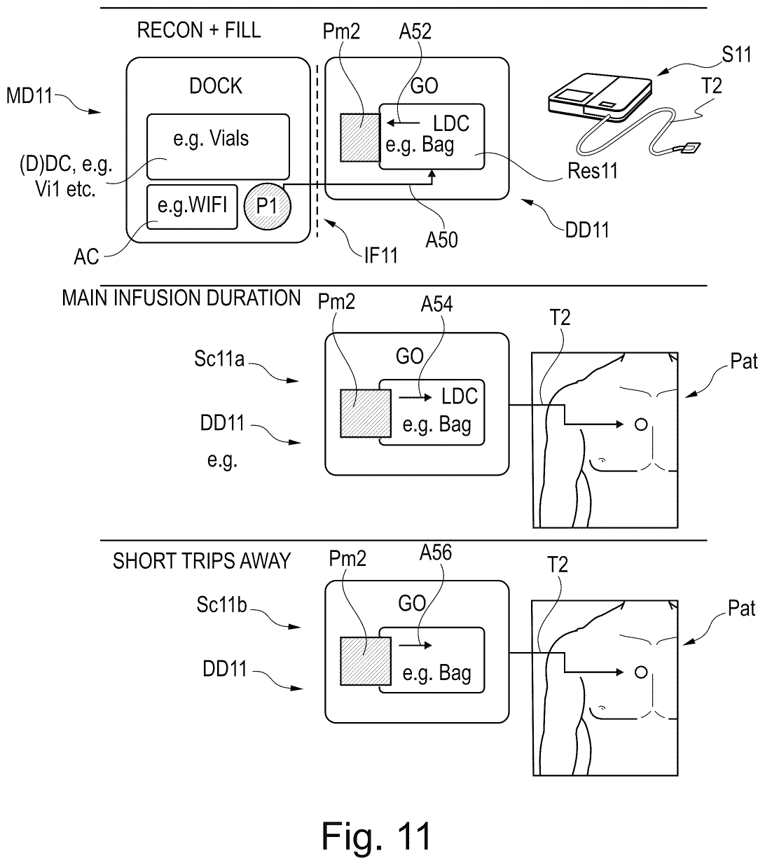

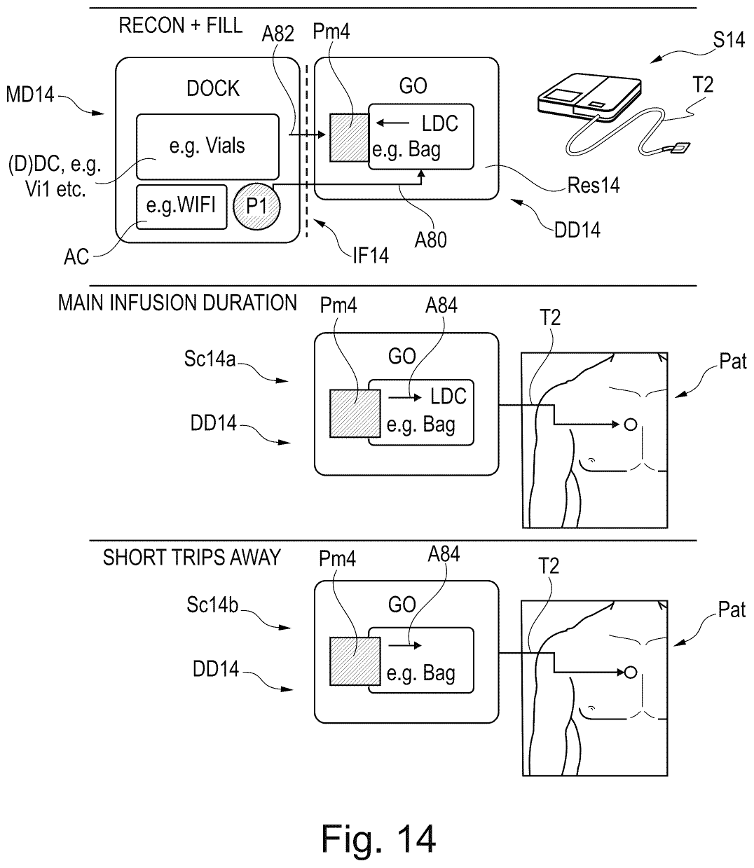

Instead of pump P2 within drug delivery device DD8, a force generating means as mentioned below with regard to the embodiments of FIGS. 11 to 13 may be used, e.g. using a mechanically driven pump which is loaded e.g. when the reservoir Res8 is filled and/or during transfer of drug D from the medical device MD8 to the drug delivery device DD8. Moreover, a combination of the embodiment of FIG. 8 with the embodiment(s) of FIGS. 14 and 15 is possible as well, e.g. using a mechanically driven pump which is loaded via a separate loading mechanism which is different from the drug transfer.

Further with reference to FIG. 8, the medical device MD, MD1, MD8 may comprise a retaining space RS that may be configured to retain or that may retain a plurality of drug containers DC at once. The medical device MD1, MD8 may be configured to handle the drugs D in all drug containers DC of the plurality of drug containers DC during a drug handling procedure. The drug delivery device DD1, DD8 may comprise a drug container LDC or the medical device MD1 may comprise a drug container MDC, see FIG. 9, comprising a volume that is sufficient to accommodate all the drugs D that are handled during the drug handling procedure.

In the system S, S1, etc., the medical device MD, MD1, MD8 may be configured to transfer the drugs D handled during the drug handling step into the drug container LDC of the drug delivery device DD1, DD8. The drug delivery device DD1, DD8 may comprise a drug container LDC comprising a volume that is sufficient to accommodate all the drugs D that are handled during the drug handling procedure, e.g. a volume that is at most 10 percent larger than required.

FIG. 9 illustrates usage scenarios of the infusion pump module according to a first alternative embodiment.

A system S9 may comprise:
A medical device MD9, e.g. similar to medical device MD, MD1, etc., and/or
A drug delivery device DD9, e.g. similar to drug delivery device DD, DD1, etc.

Thus, the medical device MD9 may comprise:
A pump P1, and/or
An auxiliary container AC (e.g. WFI), and/or
A (dry) drug container(s) (D)DC, e.g. vial Vi1 to Vi3 or Vi1 to Vi4 or Vi1 to Vi12, and/or
A medical device drug container MDC.

Furthermore, the drug delivery device DD9 may comprise:
An optional pump P2, and/or
A reservoir Res9 corresponding to the liquid drug container LDC, e.g. a flexible bag, and/or
An optional battery Ba, e.g. a rechargeable battery Ba.

A tube T2 may be fluidically coupled to an output port of the drug delivery device DD9. An interface IF9 may be used between the medical device MD, MD1, MD9, etc. and the drug delivery device DD, DD1, DD9, etc. The interface IF9 may comprise a fluidic interface FIF and/or power interface PIF, see arrow Pow and/or other interfaces as mentioned above, e.g. a data transfer interface. The fluidic interface of the interface IF9 may be established between the medical device drug container MDC and reservoir Res9.

A reconstitution of drugs and a fill procedure of the medical device drug container MDC may be performed in one step, e.g. within intermediate docking and/or undocking of device DD9, see arrow A24 indicating that pump P1 is used to fill the medical device drug container MDC. During the reconstitution and fill step, the device DD9 may be coupled (docked) to the medical device MD9. Alternatively, the device DD9 may not be coupled (docked) to the medical device MD9 during the reconstitution and fill step, e.g. it may still be used for drug delivery.

The reservoir Res9 may be filled after the medical device drug container MDC has been filled, see arrow A26 or during filling of container MDC. The device DD9 is docked to the medical device MD in order to enable drug transfer from drug container MDC to the reservoir Res9. The maximum filling value of reservoir Res9 may be less than the maximum filling volume of the drug container MDC, see e.g. examples given in the introductory part of this document. The drug delivery device DD9 may generate a demand command directed to the medical device in order to start drug transfer, see arrow A26.

Alternatively or additionally, medical device MD9 may detect the presence of the device DD9, e.g. the docked state thereof. Medical device MD9 may start filling of reservoir Res9 automatically if device DD9 is docked onto device MD9, see arrow A26. The filling level of reservoir Res9 may be detected, e.g. by device MD9 or by device DD9. In the latter case, device DD) may communicate the filling level to device MD9.

A usage scenario SC9a may be valid for a main infusion duration. In usage scenario SC9a, the device DD9 may be docked onto device MD9, e.g. there may be no undocking for the time of the main infusion duration. As soon as reservoir Res9 is emptied, filling of the reservoir Res9 may be triggered as mentioned above. Thereafter, reservoir Res9 may be filled with drug(s) solution which is already within the medical device drug container MDC, see arrow A28, e.g. without performing a new reconstitution/mixing step. This may be repeated until the medical device drug container MDC is emptied.

The main infusion may take place when the patient Pat is sleeping and or when he does not move around and/or move away from medical device MD9.

An optional electronic control unit ECU2 may detect the fill level of reservoir Res9, e.g. using a fill level detector sensor. The electronic control unit ECU2 may signal to ECU1 the filling level

Alternatively, the time of operation of pump P2 may be detected or measured in order to trace the filling level of reservoir Res9.

The same may apply to the detection of the filling level in medical device drug container MDC.

A usage scenario Sc9b may be valid for short trips away from medical device MD9. Medical device MD may be a device that is operated on a network of a utility provider, e.g. using a power plug. Thus, medical device may not comprise a battery, e.g. not a rechargeable battery.

Even if the medical device MD9 comprises such a battery it may be to heavy or too bulky to be carried around by patient Pat. The same may be valid for medical device MD8 as mentioned above and for medical device MD10 as mentioned below. In usage scenario Sc9b, device DD9 may be undocked from device MD9. Thus, the user may carry the small device DD9 around during infusion or other type of drug administration. Infusion may be started on demand of the patient Pat and/or automatically when necessary.

The smaller reservoir Res9 may reduce the size of the drug delivery device DD9. Thus, drug delivery device DD9 may be suitable for short trips away from primary infusion location.

Scenario SC9b may correspond to scenario b) mentioned above, i.e. to the “pit stop” scenario.

The medical device drug container MDC (e.g. a bag) may be changed after it is empty and before the next drug handling procedure is performed, e.g. for the same patient Pat or for a different patient Pat. Medical device drug container MDC (e.g. a bag) may be changed e.g. together with fluidic system FGS of medical device MD9.

Reservoir Res9 may be e.g. a flexible bag. Reservoir Res9 may be changed after each use, e.g. if it is again empty or at the latest when the MDC is changed. Reservoir Res9 may be changed e.g. together with fluidic system FGS2.

It may be beneficial to reduce the size and weight of the drug delivery device DD, DD1, DD9 (e.g. comprising a pump) to give the user freedom to move around and do activities during infusion, which can sometimes last several hours. For smaller infusion volumes, this may be possible using elastomeric pumps, syringe drivers or infusion pumps. However, for larger infusion volumes (e.g. can be up to about 700 ml (milliliter), this may get more challenging as the fluid reservoir (usually an IV (intravenous) bag) may be large and heavy.

The alternative embodiments described may aim to enable a small and portable drug administration (e.g. infusion) device, e.g. drug delivery device DD, DD1, DD9 even for large infusion volumes, by using a partial dose reservoir in the detachable Go pumping subsystem and refilling this using the fixed Dock drug preparation subsystem, e.g. the medical device MD, MD1, MD9, etc. with minimal user effort. This may require a quick, sterile and re-connectable fluid connection between the devices of the system.

Thus, it may not be necessary to use several smaller fluid reservoirs to achieve a full dose. This may substantially reduce the waste, i.e. compared to the case in which multiple elastomeric pumps or IV bags are used. Moreover, this may not require repeated partial dose drug preparation to fill the reservoir throughout the treatment. Manual partial drug preparation may not be desirable as it may introduce a higher risk of sterility issues.

Instead of pump P2 a force generating means as mentioned below with regard to the embodiments of FIGS. 11 to 13 may be used. Moreover, a combination of the embodiment of FIG. 9 with the embodiment(s) of FIGS. 14 and 15 is possible as well, e.g. using a mechanically driven pump which is loaded e.g. each time the reservoir Res9 is filled.

Further with reference to FIG. 9, the medical device MD, MD1, MD9, etc. may comprise a drug container MDC comprising a first volume that is sufficient to accommodate all the drugs D that are handled during the drug handling procedure. The medical device MD, MD1, MD9, etc. may be configured to dispense the drug D from the drug container MDC in at least two separate or in at least three separate dispense procedures, e.g. on demand. The drug delivery device DD1, DD9 may comprise a drug container Res9 comprising a second volume, optionally a maximal filling volume. The second volume may be less than 60 percent of the first volume or less than 30 percent of the first volume.

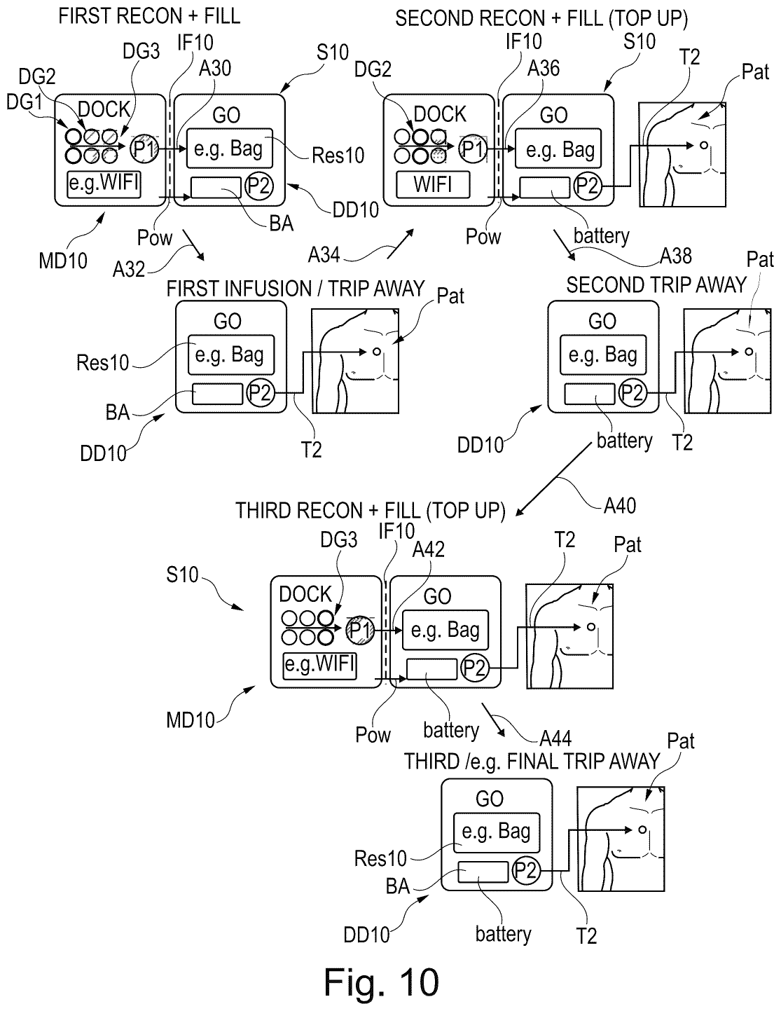

FIG. 10 illustrates usage scenarios of the infusion pump module according to a “made to order” embodiment, e.g. scenario c) as mentioned above.

A system S10 may comprise:
A medical device MD10, e.g. similar to medical device MD, MD1, etc., and/or
A drug delivery device DD10, e.g. similar to drug delivery device DD, DD1, etc.

Thus, the medical device MD10 may comprise:
A pump P1, and/or
An auxiliary container AC (e.g. WFI), and/or
A (dry) drug container(s) (D)DC, e.g. vial Vi1 to Vi3 or Vi1 to Vi4 or Vi1 to Vi12, e.g. forming at least two groups DG1 and DG2 or forming at least three groups DG1, DG2 and DG3.

Medical device MD10 may not comprise a drug container for the handled/prepared drugs.

Furthermore, the drug delivery device DD10 may comprise:
An optional pump P2, and/or
A reservoir Res10, e.g. a flexible bag, and/or
An optional battery Ba, e.g. a rechargeable battery Ba.

A tube T2 may be fluidically coupled to an output port of the drug delivery device DD10. An interface IF10 may be used between the medical device MD, MD1, MD10, etc. and the drug delivery device DD, DD1, DD10 etc. The interface IF10 may comprise a fluidic interface FIF and/or power interface PIF, see arrow Pow and/or other interfaces as mentioned above, e.g. a data transfer interface. The fluidic interface of the interface IF10 may be established between the fluid guide system of the medical device MD10 and the liquid drug container LDC of the drug delivery device DD10. Thus, the medical device MD10 may not comprise a liquid drug container which stores the drug(s) after drug handling, e.g. after reconstitution and/or drug mixing.

A reconstitution and fill procedure may be performed in the docked state of the drug delivery device DD10 to the medical device MD10 on demand, e.g. in several steps with intermediated undocking of device DD10 from device MD10. Only the drugs of a specific group DG1 or DG2 or DG3, etc. of drug containers DC may be used during each drug handling step, e.g. reconstitution and/or drug mixing.

Thus, the reservoir Res10 may be filled several times until the drugs D of all groups DG1, DG2, etc. of drug containers have been used. Alternatively, several reservoirs Res10 may be used until the drugs D of all groups DG1, DG2, etc. of drug containers have been used. Thus, the maximum filling volume of reservoir Res10 may be much smaller than the filling volume which would be needed for handling the complete drug solution which is get from the drugs within the drug containers of all groups DG1, DG2, etc. Values for the volume of reservoir Res10 relative to the overall volume of drug solutions are mentioned in the introductory part of the description.

In short, the following steps may be performed as illustrated in FIG. 10:
Arrow 30: Device DD10 is docked onto device MD10. The first charge of drugs is handled, e.g. the drugs of drug container group DG1, e.g. comprising two vials. The drug solution is transferred from the device MD10 to the device DD10, e.g. into reservoir Res10, and/or
Arrow 32: Thereafter, device DD10 is undocked from device MD10. The drugs within reservoir Res10 are delivered to patient Pat, and/or
Arrow 34: Then, after the reservoir Res10 is empty, device DD10 is docked onto device MD10 again. This may be detected by the device MD10 and may trigger a second cycle of drug handling, e.g. of the drugs in the drug containers of group DG2, and/or
Arrow 36: During drug handling the generated drug solution is transferred from medical device MD10 to device DD10, e.g. using pump P1, and/or
Arrow 38: Thereafter, the device DD10 is undocked and may be used for a second cycle of drug delivery. The scenario may end after two cycles of drug handling and drug delivery, and/or
Arrow 40: An optional third cycle of drug handling may be started on demand, e.g. by docking device DD10 again onto device MD10.
Arrow 42: The third cycle of drug handling may be performed and during this cycle the drug solution may be transferred to the device DD10, e.g. using pump P1. Drugs within drug containers of the third group DG3 may be used for drug handling, e.g. reconstitution and/or drug mixing.
Arrow 44: Thereafter, device DD10 may be undocked again and may be used for drug delivery.

Reservoir Res 10 (e.g.) bag may be changed after each cycle, e.g. together with fluid guide system FGS2 of DD10. Alternatively, reservoir Res 10 (e.g.) bag may be changed only after the delivery of all of the drugs within medical device MD10. Reservoir Res 10 (e.g. bag) may be changed together with the fluid guide system FGS2 of device DD10.

The fluid guide system FGS of device MD10 may be changed if all drugs D within medical device MD10 have been handled.

The filling level within reservoir Res10 may be detected, e.g. using a fill sensor and/or monitoring the infusion time, e.g. the time for which the pump P2 is switched on.

Instead of the electrical pump P2, a force generating device according to one of the embodiments of FIGS. 11 to 13 or a purely mechanical pumping device of the embodiment(s) of FIGS. 14 and/or 15 may be used.

Further with reference to FIG. 10, the medical device MD, MD1, MD10, etc. may comprise a retaining space RS for a plurality of drug containers DC. The medical device MD, MD1, MD10, etc. may be configured to handle at least one drug D in a first group DG1 of at least one drug container DC or of at least two drug containers DC of the plurality of drug containers DC during a first drug handling procedure, optionally on demand. The drug(s) of the first group DG1 may be transferred to the drug delivery device DD1, DD10 during drug handling. After the delivery of the drugs D of the first group into the body B of a patient Pat, the medical device MD, MD1, etc. may handle at least one drug D in a second group DG2 of at least one drug container DC or of at least two drug containers DC of the plurality of drug containers DC optionally on demand during a second drug handling procedure. The drug(s) of the second group DG2 may be transferred to the drug delivery device DD1, DD10 during the second drug handling procedure.

FIG. 11 illustrates usage scenarios of the infusion pump module IPM (DD) according to a second alternative embodiment, variant A.

The scenarios which are illustrated in FIG. 11 are very similar to the scenarios which are illustrated in FIG. 8. Therefore, mainly the differences are highlighted in the following. A force generating unit within the drug delivery device DD may be charged by fluid input/inflow from the medical device MD.

Thus, there may be the following corresponding items:
A system S11 may correspond to the system S8, and/or
A medical device MD11 may correspond to the medical device MD8, and/or
A drug delivery device DD11 may correspond to the drug delivery device DD8.

A pump P1 of medical device MD11 may be more powerful compared to the pump P1 of medical device MD8 because in system S11 the pressure of the fluid transfer from medical device MD11 to drug delivery device DD11 is used to load the purely mechanical pump Pm2 within the device DD11. Examples for purely mechanical pumps/force generating device(s) are mentioned below in the description of FIG. 12 (spring driven plunger) and of FIG. 13 (elastomeric pump).

The medical device MD11 may further comprise:
An auxiliary container AC (e.g. WFI), e.g. the same as in device MD8, and/or
(Dry) drug containers (D)DC, e.g. vial Vi1 to Vi3 or Vi1 to Vi4 or Vi1 to Vi12, e.g. the same as in device MD8.

The drug delivery device DD11 may comprise:
The purely mechanically driven pump Pm2 (pump drive mechanism) which may be also named as a force generating means, and
A liquid drug container LDC (e.g. bag), e.g. similar as in device MD8,

The device DD11 may not comprise a battery Ba, e.g. no rechargeable battery. Thus, an interface IF11 which corresponds to interface IF8 may only comprise a fluidic interface but no power interface and/or no communication interface. However, alternatively, a battery within drug delivery device DD11 and/or a power interface may be used, e.g. in order to enable the data communication between device DD11 and MD11 as mentioned above, see introductory part of the description and/or description of FIG. 3.

Again a tube T2 may be used to transfer drug fluid out of the liquid drug container LDC or out of a reservoir Res11 (e.g. bag) into the body of the patient Pat.

A reconstitution within medical device MD11 and a fill step of the liquid drug container LDC in the drug delivery device DD11 may be the same as mentioned above with relation to FIG. 8, medical device MD8 and drug delivery device DD8, see arrow 50 which may correspond to arrow 24. An arrow A52 illustrates the pressure of the drug solution which loads the purely mechanical pump Pm2, see also the detailed embodiments of FIGS. 12 and 13 as described below.

A usage scenario Sc11a may be valid for a main infusion duration. The usage scenario Sc11a may correspond to usage scenario Sc8a as mentioned above, see description of FIG. 8. An arrow A54 illustrates the force that is provided by the purely mechanical pump Pm2/force generating unit for delivery of the drug D out of the liquid drug container LDC of the drug delivery device DD11 through the tube T2 into the body B of the patient Pat.

A usage scenario Sc11b may be valid for short trips away from medical device MD11, e.g. away from home. The usage scenario Sc11b may correspond to the usage scenario Sc8b as mentioned above, see description of FIG. 8. Again, an arrow A56 illustrates the force that is provided by the purely mechanical pump Pm2/force generating unit for delivery of the drug D out of the liquid drug container LDC of the drug delivery device DD11 through the tube T2 into the body B of the patient Pat.

Different layouts based on pump location and/or reservoir sizes may be used to optimize the size of the drug delivery device, e.g. DD11 and/or the size of the pump Pm2/force generating unit in the drug delivery device, e.g. DD11. Another option which could e.g. be used in parallel with a partial dose reservoir to further reduce the size and weight of the Go infusion pump may be to replace the electro-mechanical pumping system by a purely mechanical system.

These embodiments may take advantage of having an electromechanical unit in the medical device MD (Dock) to recharge the drug delivery device (Go) mechanical energy before use. Low levels of waste may be reached by multiple use of an elastomeric pumps. There may be no need for the user to mechanical recharge the pump/force generating member, e.g. an elastomeric pump filling or a spring priming of a spring based reusable system.

The mechanical energy storage and release mechanism may be recharged directly by filling of the drug delivery device (pump), see FIGS. 11 to 13 or the medical device MD (dock) may actuate a mechanism within the pumping sub-system which may store the mechanical energy and may use it for pumping/drug delivery, see FIGS. 14 and 15.

The drug delivery device DD11 may be modified by using a reservoir, e.g. Res11 which may have a much smaller volume compared to the volume (capacity) of the liquid drug container LDC. Same scenarios as mentioned above for FIGS. 9 and 10 may be applied, i.e. “pit stop” or “made to order”.

A medical device corresponding to the medical device MD9 (e.g. comprising a medical device drug container MDC may be able to store the whole amount of drugs handled in the drug handling process) or corresponding to the medical device MD10 (e.g. being able to handle drugs of groups DG1, DG2, etc. of drug containers) may use a more powerful pump P1 in order to load the purely mechanical pump Pm2 in the drug delivery device via the pressure used for the transfer of the liquid drug(s). Thus, there may be no power interface or at least no power interface used for generating the force(s) for drug delivery.

The change of the fluid guide systems FGS, FGS2 and/or of the drug containers LDC may be as mentioned above for FIG. 8. For the modified drug delivery device MD11, the change of the fluid guide systems FGS, FGS2 and/or of the drug containers MDC may be as mentioned in the description of FIG. 9 or 10.

Further with reference to FIG. 11 the drug delivery device DD, DD1, DD11 may be configured to receive mechanical energy from the medical device MD, MD1, MD11. The drug delivery device DD1, DD11 may comprises a storage element or may be configured to comprise a storage element. The storage element may be configured to store the received mechanical energy.

The drug delivery device DD, DD1, D11 may be configured to receive the mechanical energy during the transfer of the at least one drug D from the medical device MD, MD1, MD11 to the drug delivery device DD1 via the generated fluid pressure of a fluid comprising the at least one drug D. The drug delivery device DD1 may be configured such that the fluid pressure loads the mechanical storage element. Optionally, the mechanical storage element may comprise at least one mechanical spring Sp12 that may be configured to store the transferred energy, e.g. a helical compression or a helical tension spring. According to another embodiment, the mechanical storage element may be an inflatable drug container DC that is configured to be inflated by the pressure.

FIG. 12 illustrates an embodiment using a spring driven plunger PI.

Thus, non-electronic dock-side powering means may be used to transfer mechanical energy to the drug delivery device, e.g. to a drug delivery device DD12 as illustrated in FIG. 12. The driver for this concept may be the aim to avoid an electrical mechanical pump in the drug delivery device, e.g. to a drug delivery device DD12 (Go device), e.g. in order to reduce the size and/or the costs of the unit and/or of the overall system S, S1, S12, etc. Thus, filling the drug delivery device, e.g. to a drug delivery device DD12 (Go) with the drug solution may push back a plunger PI which may load a spring, e.g. Sp12.

A system S12 may comprise:
A medical device MD12, and/or
A drug delivery device DD12.

The medical device MD12 may correspond to medical system MD11 or to any other of the medical systems MD, MD1 etc. as mentioned above. The medical device MD12 may comprise:
A pump P1, e.g. a comparably powerful pump, and/or
An output channel OCH, and/or
An output port OP12.

A connection Con12a, e.g. a fluidically connection may be established between the output port OP12 and a further fluidic port as mentioned below.

The drug delivery device DD12 may correspond to the drug delivery device DD11 or to any other of the drug delivery device DD, DD1 etc. as mentioned above. The drug delivery device DD12 may comprise:
A body B12, and/or
A retaining space RS12, e.g. for retaining several components (e.g. reservoir Res12/LDC), and/or
A reservoir Res12 (e.g. rigid and/or cylindrical) or a liquid drug container LDC which has a greater liquid storage capacity compared to reservoir Res12 as mentioned several times above, and/or
An input port IP12 which may be part of the connection Con12a, and/or
Tubing T12a (e.g. rigid and/or flexible), e.g. connecting the input port IP12 and the reservoir Res12/LDC, and/or
A spring Sp12, e.g. a helical spring and/or a compression spring, and/or
The plunger PI that was already mentioned, and/or
A plunger holding element PHE, e.g. an element that enables a sliding/translator movement of the plunger in the distal direction to deliver the drug D and in the proximal direction to load the spring Sp12 again, and/or
An optional electromechanical valve EMV, and/or
An optional electronic control unit, e.g. to control the valve EMV and/or to communicate electronic data from or to the drug delivery device DD12, and/or
An output port OP12b, and/or
Tubing T12b (e.g. rigid and/or flexible), e.g. connecting the reservoir Res12/LDC and the output port OP12. Valve EMV may act on tubing T12b, to allow or to prevent fluid flow there through.

No separate carrier may be used to carry tubing T12a and T12a. In an embodiment, as separate carrier may be used to carry tubing T12a and T12a. The separate carrier may be separate to the body B12 and separate to the reservoir Res12.

A connection Con12b may be established between the output port OP12b and the tube T2 (not illustrated in FIG. 12 but see e.g. FIG. 11).

A plug PLU, e.g. an elastomeric plug may be used within reservoir Res12/LDC. The plug PLU may slide within the reservoir Res12/LDC, e.g. in order to prevent contact between the plunger PL and the drug D and/or to provide a fluid tight closure of the open end of the reservoir Res12/LDC.

Thus, the following fluid flow may be established during filling of reservoir Res12/LDC:
Arrow A60: Prepared drug D (e.g. reconstituted and/or mixed as described above, see e.g. FIGS. 5 and 6) comes from medical device MD12, and/or
Arrows A62, A64: The pump P1 may pump the drug (solution) D out of medical device MD12, and/or
Arrow A66: The drug (solution) D flows through tubing T12a into the reservoir Res12/LDC, and/or
Arrow A68: The pressure of the drug (solution) D is sufficient to push plug PLU and plunger PL proximally and to load spring SP12 thereby.

The valve EMV may be controlled by an electronic unit of the drug delivery device DD12, e.g. in order to start drug delivery when needed by the patient Pat, e.g. on manual request of the patient and/or automatically at a pre-defined time or at pre-defined time intervals.

Without the valve EMV, drug delivery may start immediately after filling or may be started manually. The rate of drug delivery may be adjusted by using appropriate design parameters, e.g. spring constant of spring SP12, diameters of tubing T12b and/or usage of a flow regulator, e.g. a flow regulator that can be adjusted manually.

During drug delivery, the spring SP12 may unload and may push the plunger PL and the plug PLU distally. Drug (solution) D may flow out of reservoir Res12/LDC and through tubing T12b into tube T2 and further into the body b of the patient, e.g. using a catheter.

FIG. 13 illustrates an embodiment using an elastomeric pump

Again, a on electronic dock-side powering means may be used to transfer mechanical energy during the transfer of the drug (solution) D. The driver for this concept may be the aim to avoid an electrical mechanical pump in the Go device, e.g. drug delivery device DD, DD1, DD13, etc. This may enable to reduce the size and/or the costs of the drug delivery device and/or of the overall system. Filling the drug delivery device, e.g. DD13 (Go) with the drug solution may deform and load an elastomeric pump as is described in the following.

A system S13 may comprise:
A medical device MD13, and/or
A drug delivery device DD13.

The medical device MD13 may correspond to medical system MD11 or to any other of the medical systems MD, MD1 etc. as mentioned above. The medical device MD13 may comprise:
A pump P1, and/or
An output channel OCH, see e.g. output channel(s) as mentioned above (FIGS. 4 to 6), and/or
An output port OP13a.

A fluidic connection Con13a may be established between the output port and a further fluidic port as mentioned below when detachable drug delivery device DD13 is docked to medical device MD13.

The drug delivery device DD13 may correspond to drug delivery device DD11 or to any other of the drug delivery devices DD, DD1 etc. as mentioned above. The drug delivery device DD13 may comprise:
A body B13, and/or
An elastomeric consumable EC forming a reservoir Res13 or a liquid drug container LDC which may have a larger liquid storing capacity compared to reservoir Res 13 as mentioned several times above, and/or
A retaining space RS13, e.g. for retaining the elastomeric consumable EC, and/or
A fluid input port IP13, e.g. connectable to output port OP13a, and/or
A tubing T13a, e.g. rigid or flexible, and/or
A tubing T13b, e.g. rigid or flexible, and/or
An output port OP13b, and/or
An optional electromechanical valve EMV, and/or
An optional electronic control unit, e.g. for controlling the valve EMV and/or for communicating data from and/to the drug delivery device DD13.

No separate carrier may be used to carry tubing T13a and T13a. In an embodiment, as separate carrier may be used to carry tubing T13a and T13a. The separate carrier may be separate to the body B13 and separate to the reservoir Res13.

A connection Con13b may be established between the output port OP13b and the tube T2 (not illustrated in FIG. 13 but see e.g. FIG. 11).

Thus, the following fluid flow may be established during filling of the reservoir Res13/LDC:
Arrow A70: Prepared drug D (e.g. reconstituted and/or mixed as described above, see e.g. FIGS. 5 and 6) comes from medical device MD13, and/or
Arrows A72, A74: The pump P1 may pump the drug (solution) D out of medical device MD13, and/or
Arrow A76: The drug (solution) D flows through tubing T13a into the reservoir Res13/LDC, and/or
Arrow A78: The pressure of the drug (solution) D may be sufficient to expand the elastomeric consumable EC.

The valve EMV may be controlled by an electronic unit of the drug delivery device DD13, e.g. in order to start drug delivery when needed by the patient Pat, e.g. on manual request of the patient and/or automatically at a pre-defined time or at pre-defined time intervals.

Without the valve EMV, drug delivery may start immediately after filling and or after manual initiation. The rate of drug delivery may be adjusted by using appropriate design parameters, e.g. elastomeric properties of elastomeric consumable EC, diameters of tubing T13b and/or usage of a flow regulator, e.g. a flow regulator that can be adjusted manually.

During drug delivery, the elastomeric consumable EC may shrink and/or deflate and may push the drug (solution) D out of reservoir Res13/LDC and through tubing T13b into tube T2.

FIG. 14 illustrates usage scenarios of the infusion pump module according to the second alternative embodiment, variant B.

The scenarios which are illustrated in FIG. 14 are very similar to the scenarios which are illustrated in FIG. 8. Therefore, mainly the differences are highlighted in the following. A mechanical storage unit may be charged independent of the fluid input/inflow from the medical device MD to the drug delivery device DD. The mechanical energy may be transferred from the medical device MD to the drug delivery device DD. One embodiment thereof is described in more detail below, see FIG. 15.

Thus, there may be the following corresponding items:
A system S14 may correspond to the system S8, and/or
A medical device MD14 may correspond to the medical device MD8, and/or
A drug delivery device DD14 may correspond to the drug delivery device DD8.

A pump P1 of medical device MD14 may be the same as the pump P1 of medical device MD8 because in system S14 no additional pressure of the fluid transfer from medical device MD14 to drug delivery device DD14 has to be provided. However, drug delivery device DD14 may comprise a purely mechanical pump Pm4 which may be loaded by the medical device MD14, e.g. independent of the fluid flow, e.g. at the same time, before and/or thereafter.

The medical device MD14 may further comprise:
An auxiliary container AC (e.g. WFI), e.g. the same as in device MD8, and/or
(Dry) drug containers (D)DC, e.g. vial Vi1 to Vi3 or Vi1 to Vi4 or Vi1 to Vi12, e.g. the same as in device MD8.

The drug delivery device DD14 may comprise:
The purely mechanically driven pump Pm4 (pump drive mechanism) which may be also named as a force generating means, see e.g. embodiment of FIG. 15, and/or
A liquid drug container LDC (e.g. bag), e.g. the same as in device MD8,

The device DD14 may not comprise a battery Ba, e.g. no rechargeable battery. Thus, an interface IF14 which corresponds to interface IF8 may only comprise a fluidic interface but no power interface and/or no communication interface. However, alternatively, a battery may be used within drug delivery device DD14 and/or a power interface may be used, e.g. in order to enable the data communication between device DD14 and MD14 as mentioned above, see introductory part of the description and/or description of FIG. 3.

Again a tube T2 may be used to transfer drug fluid out of the liquid drug container LDC or out of a reservoir Res14 (e.g. bag) into the body of the patient Pat.

A reconstitution within medical device MD14 and a fill step of the liquid drug container LDC in the drug delivery device DD14 may be the same as mentioned above with relation to FIG. 8, medical device MD8 and drug delivery device DD8. An arrow A80 illustrates the transfer of the drug solution D from medical device MD14 to drug delivery device DD14, e.g. under control of the medical device MD14 and by using the electrical (mechanical) pump P1 of the medical device MD14.

An arrow A82 symbolizes the transfer of mechanical energy from the medical device MD14 to the drug delivery device DD14. An embodiment is mentioned below in the description of FIG. 15. Other embodiments may be used as well, e.g. loading of the spring SP12 by moving the plunger PI proximally, e.g. using a tooth rack and a similar motor M and gear G, e.g. as mentioned below.

A usage scenario Sc14a may be valid for a main infusion duration. The usage scenario Sc14a may correspond to usage scenario Sc8a as mentioned above, see description of FIG. 8. An arrow A84 illustrates the force that is provided by the purely mechanical pump Pm4/force generating unit for delivery of the drug D out of the liquid drug container LDC of the drug delivery device DD14 through the tube T2 into the body B of the patient Pat.

A usage scenario Sc14b may be valid for short trips away from medical device MD14, e.g. away from home. The usage scenario Sc14b may correspond to the usage scenario Sc8b as mentioned above, see description of FIG. 8. Again, an arrow A86 illustrates the force that is provided by the purely mechanical pump Pm4/force generating unit for delivery of the drug D out of the liquid drug container LDC of the drug delivery device DD14 through the tube T2 into the body B of the patient Pat.

The drug delivery device DD14 may be modified by using a reservoir, e.g. Res14 which may have a much smaller volume compared to the volume (capacity) of the liquid drug container LDC. Same scenarios as mentioned above for FIGS. 9 and 10 may be applied, i.e. “pit stop” or “made to order”.

A medical device corresponding to the medical device MD9 (e.g. comprising a medical device drug container MDC may be able to store the whole amount of drugs handled in the drug handling process) or corresponding to the medical device MD10 (e.g. being able to handle drugs of groups DG1, DG2, etc. of drug containers) may use separate driving unit for transferring mechanical energy to drug delivery device DD14. Thus, there may be no power interface or at least no power interface used for generating the force(s) for drug delivery in system S14. An example for the driving unit is described below in the description of FIG. 15, e.g. a motor M and a gear G.

The change of the fluid guide systems FGS, FGS2 and/or of the drug containers LDC may be as mentioned above for FIG. 8. For the modified drug delivery device MD14, the change of the fluid guide systems FGS, FGS2 and/or of the drug container(s) MDC may be as mentioned in the description of FIG. 9 or 10.

Further with reference to FIG. 14, the drug delivery device DD, DD1, DD14, DD15 may be configured to receive mechanical energy from the medical device MD, MD1, MD14, MD15. The drug delivery device DD1, DD14, DD15 may comprises a storage element, e.g. a coil spring CS or may be configured to comprise a storage element, e.g. a coil spring CS. The storage element may be configured to store the received mechanical energy.

The drug delivery device DD, DD1, DD14, DD15 may be configured to receive the mechanical energy via a movable or rotatable solid element RE that may be configured to be driven by a driving element in the medical device MD15. The solid element RE may be mechanically coupled to the storage element, e.g. coil spring CS in order to transfer movement or rotation from the solid element RE to the storage element. The storage element may comprise at least one spring that may be configured to be biased by a movement of the solid element RE. The storage element, e.g. coil spring CS may be configured as a pump or to drive a pump.

FIG. 15 illustrates an embodiment with mechanical charging and re-charging of the pump using a coil spring.

A non-electronic dock-side powering means may be used to transfer mechanical energy to the side of the drug delivery device DD. The driver for this concept may be the aim to avoid an electrical mechanical pump in the drug delivery device DD (Go device), e.g. in order to reduce the size and/or the costs of the unit and/or of the overall system.

A system S15 may comprise:
A medical device MD15, e.g. similar to medical device MD, MD1, etc., and/or
A drug delivery device DD15, e.g. similar to drug delivery DD, DD1, etc.

A mechanical interface MIF may be comprised in the interface IF between the medical device MD15 and the drug delivery device DD15. As mentioned above, the interface IF enables detaching (undocking) and attaching (docking) of the drug delivery device DD15 to/from the medical device MD15.

The mechanical interface MIF may comprise on the side of the medical device MD15:
A motor M, and
A gear G, comprising at least one or at least two toothed wheels, e.g. a gear box.

The mechanical interface MIF may comprise on the side of the drug delivery device DD15:
A cylindrical ring element RE, and/or
A coil spring CS that is fastened with its outer end to the cylindrical ring element RE.

The inner end of the coil spring CS may be attached to a housing of the drug delivery device DD15. The cylindrical ring element RE may have a ring like bottom portion BP. There may be teeth Te on the bottom portion BP of the cylindrical ring element RE for interacting with the teeth TE on the bottom portion BP. Moreover, there may be protrusions Pr1, Pr2, etc., e.g. cylinders or half cylinder on the cylindrical face of the cylindrical ring element RE. The cylindrical ring element RE may be mounted such that it is rotatable about a rotation axis A. The rotation axis A may be arranged perpendicular or a about perpendicular relative to a rotation axis of the motor M and/or to a rotation axis of the gear G.

The drug delivery device DD15 may further comprise tubing T. Tubing T may be arranged such around the cylindrical ring element RE that the ring element acts as a peristaltic pump onto the tubing T.

Thus, there may be the following operation during unloading of the spring CS:
Arrow A90: Motor M and gear G rotate on the side of the medical device, and/or
Arrow A91: The ring element RE is rotated by the interaction of the teeth of the gear G and of the teeth TE on the ring element RE. Thereby, the coil spring CS may be biased. There may be no fluid within the drug delivery device DD15 during loading of the coil spring CS, e.g. there may be no liquid fluid transport within the tubing T although there may be mechanical contact between the protrusions Pr1, Pr2 and the tubing T. Other mechanism may be used as well, e.g. detaching ring element RE from the tubing during loading of the coil spring CS and thereafter attaching the ring element RE again to the tubing, e.g. by a respective translation e.g. perpendicular to the rotation axis A, and/or
Arrow A92: If the coils spring Cs is released, e.g. by detaching drug delivery device DD15 from medical device MD15 or by any other release mechanism, the ring element RE may rotate freely in the opposite direction as indicated by the arrow A92, and/or
Arrow A94: In the meantime, drug (solution) D may have been transferred to the drug delivery device, e.g. into the liquid drug container LDC (not illustrated in FIG. 15 but see e.g. some of the other figures, e.g. FIGS. 4, 8, 11 and 14) or into a smaller reservoir Res (not illustrated in FIG. 15 but see e.g. some of the other figures, e.g. FIGS. 9, 10 and 14). The protrusions Pr1, Pr2, etc. may establish a fluid transport of the drug (solution) D within tubing T, e.g. in order to dispense the drug D through tubing T2 into the body of the patient Pat. Dispensing of the drug may comprise an infusion, e.g. an intravenous IV infusion or another intravascular infusion.

An arrow A96 illustrates that the ring element RE is illustrated in a zoomed perspective and rotated about 180 degrees compared to the installation position as shown in FIG. 15 such that the bottom portion BE of the ring element RE is visible.

Thus, an electromechanical Go (e.g. drug delivery device DD15) recharge device may be used within the Dock (medical device MD15). A mechanical energy storage system may be used within the reusable Go (e.g. drug delivery device DD15). The mechanical storage system may be spring powered, e.g. the Dock (e.g. medical device MD15) may have a mechanical interface to the Go (e.g. drug delivery device DD15) that winds up a spring mechanism in the “Go device” when the “Go device” is docked to the medical device MD, e.g. MD14, MD15.

In summary, the go unit/device may be mechanically re-charged. The motor M may turn a toothed gear G which may wind back the spring, e.g. the coils spring CS. The spring energy of the spring, e.g. CS may then be used for pumping. The pumping technology may be a peristaltic pump or the spring, e.g. the spring, e.g. CS may actuate a different (pumping) member to deliver the drug D.

In other words, a machine/system S, S1, etc. to automate the drug preparation and e.g. IV (intravenous) infusion of e.g. a lyophilized or concentrated drug product stored in drug containers, e.g. vials is provided.

The embodiments may be used to automate the procedure where otherwise multiple drug containers, e.g. vials would be required to be diluted or reconstituted and then transferred/connected to a pumping system manually by the user. This process would require many user steps and several additional consumables.

In the embodiments, a machine MD, MD1, etc. may automate drug preparation and drug delivery in a single device with minimum user inputs. This device may build on a “Auto Recon” machine by adding an integrated infusion pump and connectivity functionality. Thus, the following two applications are incorporated by reference for all legal purposes:
“Fluid guide system usable for drug handling, e.g. for reconstitution, corresponding medical device, method and computer related items”, Apr. 5, 2024, U.S. Ser. No. 18/628,338 (US attorney SNY-003-US), same as
“Fluid guide system usable for drug handling, e.g. for reconstitution, corresponding medical device, method and computer related items”, Apr. 5, 2024, EP 24168811.8 (EP attorney 989-389 EP).

### System Overview

A single system/device S, S1, etc. is proposed to automatically prepare the drug and/or pump the fluid for infusion with minimal user input. The drug delivery device DD, e.g. an infusion pump may be detachable allowing the patient Pat more freedom. The device/system S, S1, etc. may be a connectivity hub HD1, e.g. a data connectivity hub, relaying drug preparation, infusion pumping and vital sign sensing information to a single digital cloud platform CBP.

KEY DRIVERS (e.g. in addition to the Auto Recon Device/Machine):
TIME SAVINGS: Filling multiple vials or other drug containers may be performed quickly rather than sequentially via manual manipulation may substantially reduce the time required for drug preparation; and/or
SAFETY: The needle stick risk may be reduced by preventing user access to vial access spikes, and/or
STERILITY: Fewer manual handling steps may reduce the risk of contamination, and/or
EASE OF USE: Fluid transfer and dosing may be automated, reducing the time, effort and dexterity effort of the user, and/or
EFFICACY: Manual user errors in dosing by automating the process may be removed, and/or
AVOID DRUG DAMAGE: Diluent flow may be controlled to avoid jetting onto the drug, and/or
MINIMISE WASTE: Removes the need for transfer syringes and/or needles and associated packaging, replacing them with a single consumable part.
QUALITY OF LIFE: By having a fixed base system (Dock, medical device MD, MD1, etc.) and a detachable drug delivery device DD, DD1, etc. (pumping unit (Go)) there may be shared components between Dock (drug preparation subsystem) and the Go (e.g. infusion pump) and partial dose reservoirs Res may be possible allowing the Go infusion pump to be miniaturized and highly portable.
EASE OF USE: Integrating the drug preparation and pumping subsystem into a single device, may enable a single main user interface which reduces the learning curve and complexity for the user. Automated download of treatment data may reduce setup time and automatic upload of treatment to EHRs (electronic health record) enabled by connectivity features may reduce HCP effort. Automated heparin and saline flushing at the start/end of treatment may reduce the manual user steps.
REDUCED FOOTPRINT: The Dock may contain connectivity hardware to transmit data to and from the cloud to enable remote set up, troubleshooting, monitoring and automated health record updates. It may be connected to the drug delivery device, e.g. pump and vital signs monitoring equipment via e.g. low power communication protocols. This device may act as a single hub for the drug delivery device, e.g. pump and/or all vital signs monitors, reducing e.g. the number of components the user may need in their home without relying on the users' phones, e.g. cell phones or smart phones.
REDUCED RISKS: A connected monitoring device may allow monitoring of the patient's vital signs/parameters to spot adverse effects early before they become major problems. The same may apply to monitoring of the drug delivery by the drug delivery device.

A single system/device is provided to automatically prepare the drug and pump the fluid for IV infusion with minimal user input. The drug delivery device, e.g. am infusion pump may be detachable from the medical device MD, MD1, etc. allowing the patient Pat freedom. The medical device MD, MD1, etc. may be a connectivity hub, relaying drug preparation, infusion pumping and vital sign sensing information to a single digital cloud platform. It has the following high-level features:

### Automated Fluid Control

A fluid (guide) system may be used comprising e.g. electromechanical pump(s) and valves within the reusable device, to transfer the correct amount of diluent to the drug containers, e.g. vials, reconstitute/dilute and transfer the drug solution to the IV (intravenous) carrier (e.g. within the drug delivery device) or another appropriate drug container, e.g. for drug mixing.

### Sterile Consumbale

A sterile consumable may be loaded into the medical device and/or into the drug delivery device before each use, this may be the only part that contacts the fluid. It may contain interfaces to the drug vials, diluent, IV (intravenous) carrier (e.g. within the drug delivery device) and the fluid control system.

### Portable Pumping Unit

A detachable miniaturized pumping unit may be used. The pumping unit may be connected to the Dock for filling of the prepared drug solution. The pumping unit may then be detached and carried around by the user during their infusion. The pumping unit may returned to the Dock at the end of treatment. Automatic saline and/or heparin flushing may be used.

### Automated Drug Preparation

Building on the Auto-Recon Machine technology, this device uses electro-mechanical pumps and valves to prepare the drug solution from drug and diluent vials.

### Connectivity HUB

The Dock (e.g. MD, MD1, etc.) may contain connectivity hardware to transmit data to and from the cloud CBP to enable remote set up, troubleshooting, monitoring and automated health record updates. It is connected to the pump and vital signs monitoring equipment via low power communication protocols.

### User Interface

A shared main user interface UI for the Go (e.g. pump) and the drug preparation device DPD, MD, MD1, etc. may guide the user through the steps and may prompt the user to check and confirm correct setup and/or reconstitution and/or other drug preparation. The drug delivery device, e.g. pump may contain only a basic interface to reduce size and weight of the portable unit.

An integrated infusion pump may
Simplify setup by reducing the number of interfaces and setup steps for the user, and/or
Enable infusion on the move with a miniaturized robust pump enabled by sharing components with the base unit, and/or
Reduce storage and/or power, e.g. fewer items for the user to store.

A connectivity hub—e.g. Cellular and WiFi (or Bluetooth etc.) may enable:
Remote patient monitoring, and/or
Remote dose, and/or
Remote treatment set up and/or automatic EHR (electronic health record) updates, e.g. all by using the device as a hub to connect the drug delivery device, e.g. pump, vital sign sensor VSM and/or drug preparation (device MD, MD1) to the cloud CBP.

Advanced communications and/or user interface(s) The system may be an evolution of the platform enabling an intuitive and connected user experience. Connection to vital sign monitoring may be used. Moreover, the medical device may act as the main hub for the wider system S, S1, etc.

The detachable/removable drug delivery device may enable higher user comfort, e.g. using the “made to order scenario” as described above or other scenarios, e.g. “deliver whole charge at one” and/or, “pit stop”.

### Alternative Embodiment 1—Partial Dose Reservoir—Overview

According to other embodiments IV (intravenous) drug delivery may be done by either
IV bags powered by gravity,
Elastomeric pumps that are prefilled and the relaxation of an elastomer, put under tension during filling provides the energy,
Syringe drivers—either electrically or mechanically powered. Mechanically powered devices require the user to prime them before use.
Infusion pumps—electrically powered

It may be beneficial to reduce the size and weight of the pump to give the user freedom to move around and do activities during infusion, which can sometimes last several hours. For smaller infusion volumes, this may be possible using elastomeric pumps, syringe drivers or infusion pumps. However, for larger infusion volumes (can be up to ˜700 ml (about 700 milliliter)), this may get more challenging as the fluid reservoir (e.g. an IV bag) is large and heavy. With other devices, several smaller fluid reservoirs could be used to achieve a full dose, but this would either substantially add to the waste (i.e., if multiple elastomeric pumps or IV bags are used) or would requires repeated partial dose drug preparation to fill the reservoir throughout the treatment. Manual partial drug preparation may not be desirable as it may introduce a higher risk of sterility issues.

The alternative embodiments aim to enable a small and portable infusion pump even for large infusion volumes, by using e.g. a partial dose reservoir in the detachable Go (e.g. pumping) subsystem and/or refilling this using the fixed Dock drug preparation subsystem, with minimal user effort. This may require a quick, sterile and re-connectable fluid connection between the systems.

The diagrams in FIGS. 9 and 10 show how this alternative embodiment differs from the main embodiment. FIG. 8—Primary embodiment: Go (e.g. pump) may be sufficient for full infusion, may be taken away and/or around the home.

FIG. 9: Alternative embodiment 1—Top up go pump.
An in-dock reservoir MDC may enable a light weight go component.
Main infusion duration: Fluid may be pumped while maintaining the reservoir Res within the drug delivery device filled.
Short trips away: The smaller reservoir Res may reduce the size required for travel. The smaller reservoir Res may be suitable for short trips away from primary infusion location.

The partial dose refill could either be from a reservoir, e.g. MDC of pre-prepared drugs in the Dock or it could be ‘made to order’. This may remove the need for an additional reservoir within the medical device MD, MD1, etc. and/or may reduce the total system waste, e.g. as shown in the diagram of FIG. 10.

FIG. 10: Alternative embodiment—Made to order by dock.
Reconstitution may be carried out sequentially, filling vials. Close control of drug concentration may require to ensure either that concentration in bag is always the same and/or that the flow rate is controlled to allow specific drug delivery rates.
Short trips away: The smaller reservoir Res may reduce the size required for travel. The smaller reservoir Res may be suitable for short trips away from primary infusion location.

### Alternative Embodiment 2—Non-Electro-Mechanical Pump—Overview

Another option (which could be used in parallel with a partial dose reservoir or the primary embodiment) to further reduce the size and weight of the Go (e.g. infusion pump) may be to replace the electro-mechanical pumping system by a mechanical system, e.g. a purely mechanical system. Low levels of waste (elastomeric pumps) may be reached and/or there may be no need for the user to mechanical recharge them (elastomeric pump filling or spring priming of spring based reusable systems), which may reduce user effort considerably.

The proposed embodiments take advantage of having an electromechanical Dock to recharge the GO mechanical energy before use. The mechanical energy storage and release mechanism could be recharged directly by filling of the pump or the dock may actuate a mechanism within the pumping sub-system which may store the mechanical energy and may use it for pumping, see FIGS. 11 and 14.

FIG. 11: Alternative power A—non-electric pump in go portion, e.g. charged by fluid input. FIGS. 12 and 13 illustrate two specific embodiments.

FIG. 14: Alternative power A—non-electric pump in go portion, e.g. charged by independent mechanical action. FIG. 15 illustrates one specific embodiment.

A list of features, grouped in sublevels and containing variations and alternatives is presented in the introductory part. These features and feature groups FG may be combined with any one of the embodiments of the second part of the description, e.g. with the embodiments illustrated in the figures.

Although embodiments of the present disclosure and their advantages have been described in detail, it should be understood that various changes, substitutions and alterations can be made therein without departing from the spirit and scope of the disclosure as defined by the appended claims. For example, it will be readily understood by those skilled in the art that many of the features, functions, processes and methods described herein may be varied while remaining within the scope of the present disclosure. Moreover, the scope of the present application is not intended to be limited to the particular embodiments of the system, process, manufacture, method or steps described in the present disclosure. As one of ordinary skill in the art will readily appreciate from the disclosure of the present disclosure, systems, processes, manufacture, methods or steps presently existing or to be developed later that perform substantially the same function or achieve substantially the same result as the corresponding embodiments described herein may be utilized according to the present disclosure. Accordingly, the appended claims are intended to include within their scope such systems, processes, methods or steps. The embodiments mentioned in the first part of the description may be combined with each other. The embodiments of the description of figures may also be combined with each other. Further, it is possible to combine embodiments mentioned in the first part of the description with examples of the second part of the description which relates to FIGS. 1 to 15.

1. A drug delivery device for delivering at least one drug, comprising:
a case,
at least one interface portion being part of an interface to a medical device,
a retaining space within the case, the retaining space comprising at least a portion of a fluid guide system,
wherein the case comprises at least one drug container,
wherein the at least one interface portion of the drug delivery device comprises at least one inflow port of the fluid guide system of the drug delivery device,
wherein the fluid guide system is fluidically coupled to the at least one drug container,
wherein the drug delivery device is detachably connectable to the medical device via the at least one interface portion,
wherein the drug delivery device is configured to receive the at least one drug from the medical device into the at least one drug container,
wherein the at least one drug is a reconstituted drug which has been reconstituted in the medical device,
wherein the fluid guide system and the at least one drug container are replaceable,
wherein the at least one drug container comprises at least one flexible portion or movable portion which defines at least a portion of at least one reservoir within the at least one drug container,
wherein the drug delivery device is configured to receive mechanical energy from the medical device,
wherein the drug delivery device comprises a storage element or is configured to comprise a storage element,
wherein the storage element is configured to store mechanical energy,
wherein the drug delivery device is configured such that the mechanical energy is generated in the medical device (1) by an electrically-driven pump that transfers the at least one drug to the drug delivery device, or (2) by an electric motor that transfers the mechanical energy from the medical device to the drug delivery device without using pressure of the at least one drug, and
wherein the drug delivery device is configured such that the drug delivery device delivers the at least one drug in a detached state in which the drug delivery device is detached from the medical device.

2. The drug delivery device according to claim 1, wherein the drug delivery device comprises a data transmission interface portion,
wherein the data transmission interface portion comprises a data receiving and sending unit which is configured to communicate with the medical device, and
wherein the drug delivery device is configured to receive at least one of the following data from the medical device:
amount of drug/dose to be delivered,
rate of drug delivery,
data indicating that the drug delivery device is filled,
data identifying a patient,
and/or
wherein the drug delivery device is configured to send at least one of the following data to the medical device:
confirmation data that delivery of the at least one drug has been successful,
start time and end time of the delivery of the at least one drug,
start time and duration of the delivery of the at least one drug,
data indicating an actual amount of drug delivered,
data indicating a rate of drug delivery,
data indicating a type of drug(s) delivered,
data identifying a patient,
error data indicating an error during the delivery of the at least one drug,
data demanding filling of the at least one drug container.

3. (canceled)

4. The drug delivery device according to claim 1,
wherein the case comprises a convex coupling portion which is configured to complement a shape of a concave support portion of the medical device,
wherein the convex coupling portion extends along a length that is at least half or at least three quarters of a length of the drug delivery device, and
wherein at least one or at least two of the at least one interface portion of the drug delivery device is or are arranged on the convex coupling portion.

5. The drug delivery device according to claim 1,
wherein the drug delivery device is coupled to or is configured to be coupled to a catheter or to an interface to a catheter, and
wherein the drug delivery device is configured to infuse the at least one drug into a vessel of a body of a patient.

6. (canceled)

7. The drug delivery device according to claim 1,
wherein the drug delivery device is configured such that the mechanical energy is generated in the medical device by the electrically-driven pump that transfers the at least one drug to the drug delivery device,
wherein the drug delivery device is configured to receive the mechanical energy during a transfer of the at least one drug from the medical device to the drug delivery device via a generated fluid pressure of a fluid comprising the at least one drug, and
wherein the drug delivery device is configured such that the generated fluid pressure loads the storage element.

8. The drug delivery device according to claim 1,
wherein the drug delivery device is configured such that the mechanical energy is generated in the medical device by the electric motor that transfers the mechanical energy from the medical device to the drug delivery device without using pressure of the at least one drug,
wherein the drug delivery device is configured to receive the mechanical energy from the medical device via a movable or rotatable solid element that is configured to be driven by a driving element in the medical device without using pressure of a solution comprising the at least one drug,
wherein the movable or rotatable solid element is coupled to the storage element in order to transfer movement or rotation from the movable or rotatable solid element to the storage element,
wherein the storage element comprises at least one spring that is configured to be biased by a movement or rotation of the movable or rotatable solid element, and
wherein the storage element is configured as a pump or to drive a pump.

9. A medical device for drug handling, comprising:
a fluid guide system for guiding the at least one drug during drug handling,
an electronic control unit, and
an interface portion for detachable coupling of the drug delivery device according to claim 1 to the medical device,
wherein the electronic control unit is configured to control the fluid flow within the fluid guide system of the medical device during the handling of the at least one drug, and
wherein the handling of the at least one drug comprises at least one reconstitution procedure of the at least one drug, and
wherein the at least one interface portion comprises at least one outflow fluid port which is configured to transfer the at least one drug to the drug delivery device after handling of the at least one drug.

10. A system for handling at least one drug and for delivery of the at least one drug, comprising:
a medical device for handling the least one drug, optionally a medical device according to claim 9, and
a drug delivery device for delivering the at least one drug,
wherein the medical device comprises:
at least one interface portion of the medical device for detachable coupling of the drug delivery device,
wherein the handling of the at least one drug comprises at least one reconstitution procedure of the at least one drug, and
wherein the at least one interface portion of the medical device comprises at least one outflow fluid port which is configured to transfer the at least one drug to the drug delivery device after handling of the at least one drug, and
wherein the drug delivery device comprises:
a case,
at least one interface portion of the drug delivery device being part of an interface to or of the medical device,
wherein the at least one interface portion of the drug delivery device comprises at least one inflow port of a fluid guide system of the drug delivery device, and
wherein the drug delivery device is detachably connectable to the medical device via the at least one interface portion of the drug delivery device in order to transfer the at least one drug from the medical device to the at least one drug container via the at least one inflow port.

11. The system according to claim 10,
wherein the medical device comprises a retaining space that is configured to retain or that retains a plurality of drug containers at once, and
wherein the medical device is configured to handle the drugs in all drug containers of the plurality of drug containers during a drug handling procedure, and
wherein the drug delivery device comprises a drug container or the medical device comprises a drug container comprising a volume that is sufficient to accommodate all the drugs that are handled during the drug handling procedure.

12. The system according to claim 10,
wherein the medical device is configured to transfer the drugs handled during the drug handling step into the drug container of the drug delivery device, and
wherein the drug delivery device comprises a drug container comprising a volume that is sufficient to accommodate all the drugs that are handled during the drug handling procedure.

13. The system according to claim 10,
wherein the medical device comprises a drug container comprising a first volume that is sufficient to accommodate all the drugs that are handled during the drug handling procedure, and
wherein the medical device is configured to dispense the drug from the drug container in at least two separate dispense procedures, e.g. on demand,
wherein the drug delivery device comprises a drug container comprising a second volume, and
wherein the second volume is less than 60 percent of the first volume or less than 30 percent of the first volume.

14. The system according to claim 10,
wherein the medical device comprises a retaining space for a plurality of drug containers,
wherein the medical device is configured to handle at least one drug in a first group of at least one drug container or of at least two drug containers of the plurality of drug containers during a first drug handling procedure, optionally on demand,
wherein the medical device is configured to deliver the drug(s) of the first group to the drug delivery device during the first drug handling procedure, and
wherein the medical device is configured to handle at least one drug in a second group of at least one drug container or of at least two drug containers of the plurality of drug containers on demand during a second drug handling procedure after the delivery of the drugs of the first group into the body of a patient.

15. The system according to claim 10, comprising:
a first receiving and sending unit configured to communicate with a cloud based platform via a first transmission protocol,
a second receiving and sending unit configured to communicate with the drug delivery device via a second transmission protocol that is different from the first transmission protocol, and
wherein the medical device is configured to receive data related to the handling of the at least one drug via the first receiving and sending unit from a cloud based platform and/or wherein the medical device is configured to send data related to the handling of the at least one drug via the first receiving and sending unit to the cloud based platform, and
wherein data related to the delivery of the at least one drug is received from the drug delivery device via the second receiving and sending unit and/or wherein data related to the delivery of the at least one drug is sent via the second receiving and sending unit to the drug delivery device.

16. Method for drug handling using a system according to claim 11, comprising:
performing the drug handling procedure of the at least one drug within the medical device, wherein the drug handling comprises reconstitution and/or mixing of at least one drug, and
transferring the at least one drug from the medical device to the drug delivery device.

17. A computer-implemented method comprising:
sending a request to a cloud based platform for delivery of patient related data based on at least one identification of a patient,
receiving patient related data specifying details of drug handling for the patient, handling at least one drug for the patient in at least one medical device according to the patient related data,
wherein the medical device is a device according to claim 9, and
sending data related to the delivery of the drug to at least one drug delivery device.

18. The method according to claim 17, comprising:
receiving data related to the delivery of the drug from the at least one drug delivery device,
forwarding the data received from the at least one drug delivery device or data generated based on this data to the cloud based platform.

19. The method according to claim 17, comprising:
receiving physical data of the patient from at least one monitoring device, wherein the physical data indicates at least one physical parameter of the patient before and/or during and/or after delivery of the drug,
optionally forwarding the physical data to the cloud based platform,
evaluate the physical data,
depending on the evaluation of the physical data continue or stop, e.g. interrupt the delivery of the drug.

20. The method according to claim 17, comprising:
controlling the handling of the at least one drug such that:
a) drugs in at least two drug containers are handled during drug handling and all of the handled drugs are transferred to a drug container of the drug delivery device during drug handling, wherein optionally the medical device does not comprise a drug container for storing the handled drug(s) and comprising a sufficient volume to receive the whole volume of the drug fluid resulting from the drugs in the at least two drug containers or in all drug containers connectable to the medical device,
b) drugs in at least two drug containers or in all drug containers are handled during drug handling and are transferred to a drug container within the medical device to be delivered thereafter in at least two separate steps to the drug delivery device, optionally with intermediate drug delivery, or
c) the medical device is configured to be connected to a plurality of drug containers,
at least one drug in a first group of at least one drug container or at least two drug containers of the plurality of drug containers are handled optionally on demand during a first drug handling procedure,
the drug(s) of the first group are transferred to the drug delivery device, and
thereafter, at least one drug in a second group of at least one drug container or at least two drug containers of the plurality of drug containers are handled on demand during a second drug handling procedure.

21. The drug delivery device according to claim 7, wherein the storage element comprises at least one helical metal spring.

22. The drug delivery device according to claim 7 wherein the storage element comprises a balloon pump.

23. A method of using a drug delivery device, wherein the drug delivery device comprises:
a case,
at least one interface portion being part of an interface to a medical device,
a retaining space within the case, the retaining space comprising at least a portion of a fluid guide system,
wherein the case comprises at least one drug container,
wherein the at least one interface portion of the drug delivery device comprises at least one inflow port of the fluid guide system of the drug delivery device,
wherein the fluid guide system is fluidically coupled to the at least one drug container,
wherein the drug delivery device is detachably connectable to the medical device via the at least one interface portion,
wherein the drug delivery device is configured to receive the at least one drug from the medical device into the at least one drug container,
wherein the fluid guide system and the at least one drug container are replaceable,
wherein the at least one drug container comprises at least one flexible portion or movable portion which defines at least a portion of at least one reservoir within the at least one drug container,
wherein the drug delivery device is configured to receive mechanical energy from the medical device,
wherein the drug delivery device comprises a storage element or is configured to comprise a storage element,
wherein the storage element is configured to store the mechanical energy, and
wherein the mechanical energy is generated in the medical device (1) by an electrically driven pump that transfers the at least one drug, or (2) by an electric motor that transfers the mechanical energy without using pressure of the at least on drug,
wherein the method comprises:
connecting the drug delivery device and the medical device via the at least one interface portion,
after connecting, transferring the at least one drug from the medical device to the drug delivery device via the at least one inflow port of the at least one interface portion, and
after transferring, detaching the drug delivery device from the medical device for delivery of the at least one drug.

24. The method according to claim 23, wherein the at least one drug is a reconstituted drug which is reconstituted in the medical device by mixing of a diluent and at least one lyophilized drug of the at least one drug.

25. A method of delivering at least one drug from a drug delivery device, wherein the drug delivery device comprises:
a case,
at least one interface portion being part of an interface to a medical device,
a retaining space within the case, the retaining space comprising a fluid guide system,
wherein the case comprises at least one drug container,
wherein the at least one interface portion of the drug delivery device comprises at least one inflow port of the fluid guide system of the drug delivery device,
wherein the fluid guide system is fluidically coupled to the at least one drug container,
wherein the drug delivery device is detachably connectable to the medical device via the at least one interface portion,
wherein the drug delivery device is configured to receive the at least one drug from the medical device into the at least one drug container,
wherein the at least one drug is a reconstituted drug which is reconstituted in the medical device by mixing of a diluent and at least one lyophilized drug of the at least one drug,
wherein the fluid guide system and the at least one drug container are replaceable,
wherein the at least one drug container comprises at least one flexible portion or movable portion which defines at least a portion of at least one reservoir within the at least one drug container,
wherein the drug delivery device is configured to receive mechanical energy from the medical device,
wherein the drug delivery device comprises a storage element or is configured to comprise a storage element,
wherein the storage element is configured to store the mechanical energy,
wherein the mechanical energy is generated in the medical device (1) by an electrically driven pump that transfers the at least one drug or (2) by an electric motor that transfers the mechanical energy without using pressure of the at least one drug, and wherein the method comprises:
connecting the drug delivery device and the medical device via the at least one interface portion,
after connecting, transferring the at least one drug from the medical device to the drug delivery device via the at least one inflow port of at least one interface portion,
after transferring, detaching the drug delivery device from the medical device, and
after detaching, using the stored mechanical energy for delivering of the at least one drug.

26. The drug delivery device according to claim 1,
wherein the drug delivery device is configured such that the mechanical energy is generated in the medical device by the electric motor that transfers the mechanical energy from the medical device to the drug delivery device without using pressure of the at least one drug,
wherein the drug delivery device is configured to receive the mechanical energy from the medical device via a rotatable solid element that is configured to be driven by a driving element in the medical device without using pressure of a solution comprising the at least one drug,
wherein the rotatable solid element is coupled to the storage element in order to transfer movement or rotation from the rotatable solid element to the storage element,
wherein the storage element comprises at least one spring that is configured to be biased by a rotation of the rotatable solid element, and
wherein the storage element is configured as a pump or to drive a pump.
